SEQUENCE LISTING (84 sequences)

SEQ ID NO: 1 - DNA, 180 bp, Homo sapiens
cctgcccagttcccgggagggccaaccccagccagtaagaagacctgaggcgtaggatccctgcgcaggaggttgcaggaatgcccccgctagccgcaaggttcctgctggcctgtagagctttcgtgatccccgccaagatgcgaacagtaaggtcctcgtatggatcgcagtttttgt

SEQ ID NO: 2 - DNA, 180 bp, Homo sapiens
tgtttttgatgctaggtatgcttttggaatgataagcgtagaattgcttttagtgcttttgagatgtttggttgtttttggaatgctgattgcttttgtaaggatgttggaggatgcgtttttaggatgcggagtttagaagaatgattgattttaattgggagggcttttgatttgttt

SEQ ID NO: 3 - DNA, 23 bp, Homo sapiens
aggatttttgcgtaggaggttgt

SEQ ID NO: 4 - DNA, 24 bp, Homo sapiens
acgatccatacgaaaaccttacta

SEQ ID NO: 5 - DNA, 180 bp, Homo sapiens
gccgctggtgcgaagagaagagacacgcgagcggggagacctccaaggcagcgaggcatcggacatgtgtcagcacatctggggcgcacatccgtcgagcccgaggggagatttgccggaacaattcaaactgcgatattgatcttgggggtgactgtccctggccggctgtcgggtggg

SEQ ID NO: 6 - DNA, 180 bp, Homo sapiens
gggtgggctgttggctggtttttgttagtgggggttttagttatagcgttaaatttaataaggctgtttagaggggagcttgagctgcttatatgcggggtttatatgattgtgtataggctatggagcgatggaatttttagaggggcgagcgcatagagaagagaagcgtggttgctg

SEQ ID NO: 7 - DNA, 21 bp, Homo sapiens
gagcggggagatttttaaggt

SEQ ID NO: 8 - DNA, 24 bp, Homo sapiens
aaattattccgacaaatctcccct

SEQ ID NO: 9 - DNA, 180 bp, Homo sapiens
cgctcagaagccggttcacctccttctccaccgcggcatttccaaaacaacagggacaagtctccccggctcgccgcaggcctgaccgcccagctccgccaggatttgcagagagcagcgcgctccatttgcagaaaggaaatcgagtaggtcctcgcccccgactggtgcttcttgggg

SEQ ID NO: 10 - DNA, 180 bp, Homo sapiens
ggggttttttgtggttagcttttgcttttggatgagctaaaggaaagatgtttattttgcgcgatgagagatgtttaggattgctttgatttgctagtttggatgctgcttggcttttttgaatagggataataaaatttttatggcgctatttttttttttatttggctgaagatttgc

SEQ ID NO: 11 - DNA, 28 bp, Homo sapiens
gcggtatttttaaaataatagggataag

SEQ ID NO: 12 - DNA, 24 bp, Homo sapiens
cgcgctactctctacaaatcctaa

SEQ ID NO: 13 - DNA, 180 bp, Homo sapiens
agaacaactaggcgggatgtacttttgagccctgccgggtgtctccgatcggagtctggggttgagatttgggctgcacttgtccccggtgtgtctctccggcggagtaccctgaaggtgcacgaggtggggagcataggctgaggtgggtaatcgggtcctggatagaaacacaaccct

SEQ ID NO: 14 - DNA, 180 bp, Homo sapiens
ttttaatataaagataggttttgggctaatgggtggagttggatatgaggggtggagcatgtggaagttttatgaggcggcttttttgtgtggcttttgtttatgttgggtttagagttggggtttgaggctagcttttgtgggctgttttgagtttttatgtagggcggattaataaga

SEQ ID NO: 15 - DNA, 24 bp, Homo sapiens
gcgggatgtatttttgagttttgt

SEQ ID NO: 16 - DNA, 23 bp, Homo sapiens
ctcgtacaccttcaaaatactcc

SEQ ID NO: 17 - DNA, 180 bp, Homo sapiens
gcgagccgccggggtgtgagtcagcgcgctgggggctaagaagctgggtgaatagtcacggaatctcactcacgctcggctcctccacccatcccgtctacagcgcgtgtcccagtccagggcgtgcgtgcgctcggtgtccgattccgggctgtgtgtgtccatttggcgagatgtcga

SEQ ID NO: 18 - DNA, 180 bp, Homo sapiens
agctgtagagcggtttatttgtgtgtgttgggctttagcttgtggcttgcgtgcgtgcgggatttgattttgtgcgcgatatttgctttatttatttttttggcttgcatttattttaaggcattgataagtgggttgaagaattgggggttgcgcgattgagtgtggggctgctgagcg

SEQ ID NO: 19 - DNA, 21 bp, Homo sapiens
gcgcgttgggggttaagaagt

SEQ ID NO: 20 - DNA, 23 bp, Homo sapiens
cacgcacgccctaaactaaaaca

SEQ ID NO: 21 - DNA, 120 bp, Homo sapiens
agcgccaacgtgtcggacaaggaagcgggtagcaacgagaatgacgaccagaacctgggcgccaagcggcggggaccgcgcaccaccatcaaagccaagcagctggagacgctgaaggcc

SEQ ID NO: 22 - DNA, 120 bp, Homo sapiens
ctggaagttgcagaggttgatgaattgaaattattattatgcgctaggggcggcgaattgcgggtttaagattagcagtaagagcaatgatgggcgaaggaataggctgtgcaattgcga

SEQ ID NO: 23 - DNA, 22 bp, Homo sapiens
gtcggataaggaagcgggtagt

SEQ ID NO: 24 - DNA, 31 bp, Homo sapiens
cgtctccaactacttaactttaataataata

SEQ ID NO: 25 - DNA, 180 bp, Homo sapiens
ggcgccccactaaggaggacacaggctctggtgtgtgtggtgtgcgagaccccgagctcgaggccgagccaaggctgggcagaaagttgcaatcacgtgctgtcggagcccactggagcgcacagcccgctccccctgggacgcccaggcggaggacctgctgcgccctcccagggctcg

SEQ ID NO: 26 - DNA, 180 bp, Homo sapiens
gcttgggatttttttgcgttgtttaggaggcggatttgcagggttttttttgcttgatatgcgaggttatttgaggctgttgtgcattaatgttgaaagatgggttggaattgagctggagcttgagctttagagcgtgtggtgtgtgtggttttggatataggaggaattattttgcgg

SEQ ID NO: 27 - DNA, 22 bp, Homo sapiens
cgaggtcgagttaaggttgggt

SEQ ID NO: 28 - DNA, 26 bp, Homo sapiens
accctaaaaaaacgcaacaaatcctc

SEQ ID NO: 29 - DNA, 152 bp, Homo sapiens
ccttactgcacctggggtgtgtctccgcgtggtgcagagcgcgcgctctactccggaagctacggccgggtgccgcgccaccgctgtgcgccctgggcctgatccctacgccctagtcgagtgcagggcagggcaatttcgccgtgggtcct

SEQ ID NO: 30 - DNA, 152 bp, Homo sapiens
ttttgggtgctgctttaatgggatgggatgtgagctgattttgcatttttagtttgggttttgcgtgttgctattgcgctgtgggctggcattgaaggctttattttgcgcgcgagatgtggtgcgcttttgtgtggggtttatgttatttt

SEQ ID NO: 31 - DNA, 21 bp, Homo sapiens
gggtgtgttttcgcgtggtgt

SEQ ID NO: 32 - DNA, 30 bp, Homo sapiens
tcgactaaaacgtaaaaatcaaacccaaaa

SEQ ID NO: 33 - DNA, 180 bp, Homo sapiens
cctggccagaagctccgggatcgcagccctcccgggtccggcttcatccctgcccggccaccgaggccctctttttctgcaccgcggattctcctccgcctgcgtgttcggggcccttgtatccgatgtttctttctaaaagttgtccttccggctgattcggaagtcgctccaagggaa

SEQ ID NO: 34 - DNA, 180 bp, Homo sapiens
aagggaattttgctgaaggcttagttggcttttttgttgaaaatttttttttgtagcttatgtttttggggcttgtgcgtttgcttttttttaggcgctatgttttttttttttggagctattggcttgtttttattttggcttgggcttttttgatgctagggctttgaagattggttt

SEQ ID NO: 35 - DNA, 28 bp, Homo sapiens
gttttttcgggttcggttttatttttgt

SEQ ID NO: 36 - DNA, 28 bp, Homo sapiens
ccgaatcaaccgaaaaaacaacttttaa

SEQ ID NO: 37 - DNA, 120 bp, Homo sapiens
cgctggcccctcgcggagctttccctggcgcgacctcacacggtcgctgcctctattccgaccacgctctgcttcgctggctgcggctccgccaggaatccgagggggcgcaggcccagg

SEQ ID NO: 38 - DNA, 120 bp, Homo sapiens
ggatttggatgcgggggagcttaaggattgctttggcgttggttgctttgttttgcattagctttatttttgttgctggcatattttagcgcggttttttttgaggcgctttttggttgc

SEQ ID NO: 39 - DNA, 26 bp, Homo sapiens
ggagttttttttggcgcgattttata

SEQ ID NO: 40 - DNA, 24 bp, Homo sapiens
aattcctaacgaaaccgcaaccaa

SEQ ID NO: 41 - DNA, 120 bp, Homo sapiens
ccggctccgggacacacgtggaagccgggtcctgcagcaagaggaagtccaggatcgcggcgaggagacggcggggcccgggcgcctgggctgccgtcccggctctgggtgggtgggtgg

SEQ ID NO: 42 - DNA, 120 bp, Homo sapiens
ggtgggtgggtgggttttggctttgctgttgggtttgcgggcttggggcggcagaggagcggcgctaggatttgaaggagaatgatgttttgggctgaaggtgcatatagggctttggct

SEQ ID NO: 43 - DNA, 24 bp, Homo sapiens
tatacgtggaagtcgggttttgta

SEQ ID NO: 44 - DNA, 24 bp, Homo sapiens
ccaaaaccgaaacgacaacccaaa

SEQ ID NO: 45 - DNA, 180 bp, Homo sapiens
cgctggcccctcgcggagctttccctggcgcgacctcacacggtcgctgcctctattccgaccacgctctgcttcgctggctgcggctccgccaggaatccgagggggcgcaggcccaggctcggccctagatgcgcggaatcgccatcagcctttgcttacaccagcgtggccgcaggg

SEQ ID NO: 46 - DNA, 180 bp, Homo sapiens
gggatgctggtgcgattatatttgtttttgattattgctaaggcgcgtagattttggcttggatttggatgcgggggagcttaaggattgctttggcgttggttgctttgttttgcattagctttatttttgttgctggcatattttagcgcggttttttttgaggcgctttttggttgc

SEQ ID NO: 47 - DNA, 29 bp, Homo sapiens
gttgtttttatttcgattacgttttgttt

SEQ ID NO: 48 - DNA, 28 bp, Homo sapiens
ccacgctaatataaacaaaaactaataa

SEQ ID NO: 49 - DNA, 120 bp, Homo sapiens
gcatacacacgtacacaggcaggggcagccggctccgcggcgcacatcgcggcagctcaggcaacgcaaagttggtggcgtgttccgtccaggcgctccctaccttcccaggcgcttcgc

SEQ ID NO: 50 - DNA, 120 bp, Homo sapiens
tgctttgcggatttttttattttttgcggatttgctttgtgcggtggttgaaatgcaatggatttgatggcgctatatgcggcgctttggctgatggggatggatatatgcatatatatg

SEQ ID NO: 51 - DNA, 24 bp, Homo sapiens
tatacgtatataggtaggggtagt

SEQ ID NO: 52 - DNA, 22 bp, Homo sapiens
acgaaacacgccaccaacttta

SEQ ID NO: 53 - DNA, 180 bp, Homo sapiens
ggacgcggtctgtgcgtctcctgctcagagccagaaatcagcacccgaaggcatgagactgccagttgccagcgaattcacaaatccgaccggcccctcccggcccaccgacctcgggaccgccccaggaacatattcagcactgtggccagcgccacatccatcctaccgcaaagcgcc

SEQ ID NO: 54 - DNA, 180 bp, Homo sapiens
ctgcgaaatgctattttatttatattgcgattggtgttatgatttatataaggattttgctagggctttagctatttggctttttttggctagcttaaatatttaagcgattgttgattgttagagtatggaagcttatgattaaagattgagatttgttttttgcgtgtttggcgcagg

SEQ ID NO: 55 - DNA, 30 bp, Homo sapiens
gattgttagttgttagcgaatttataaatt

SEQ ID NO: 56 - DNA, 29 bp, Homo sapiens
atataacgctaaccacaatactaaatata

SEQ ID NO: 57 - DNA, 96 bp, Homo sapiens
gcagcgcgacgctcccctctcccgaaaggttggctccacggtcccgccggccgcgcaggtctggctgaactgcttggggtcgcccggctcctctcg

SEQ ID NO: 58 - DNA, 96 bp, Homo sapiens
gctttttttggcttgctggggtttgttaagttggtttggatgcgctggctgctttggcattttggttggaaagctttttttttttgcagcgcgatg

SEQ ID NO: 59 - DNA, 30 bp, Homo sapiens
gacgtttttttttttcgaaaggttggtttt

SEQ ID NO: 60 - DNA, 25 bp, Homo sapiens
acgaccccaaacaattcaaccaaac

SEQ ID NO: 61 - DNA, 120 bp, Homo sapiens
gacggagccataaaaggggggacacgggggctggagttgcggctgctcgggccgcgccgccgccaccgccaccctggtcccacgggagccactcggagccatgccactgggtgcgcgggt

SEQ ID NO: 62 - DNA, 84 bp, Homo sapiens
tgggcgcgtgggttattgtattgaggcttattgagggcattttggttttattgctattgctgctgcgctgggcttgttggcgtt

SEQ ID NO: 63 - DNA, 20 bp, Homo sapiens
ggatacgggggttggagttg

SEQ ID NO: 64 - DNA, 21 bp, Homo sapiens
cgcgcacccaataacataact

SEQ ID NO: 65 - DNA, 180 bp, Homo sapiens
gtccttgaacccgtggcactcggtagagagagaggagatgatcggaaagtgcgtgggaacaatgtctattccgcgcgaccatagctctcacatccctaaggcgccagccttttttgaaaatccgtaacgttttgctttgtgtcccaggctgcgggcctaatagaaaacgcgccgaacttg

SEQ ID NO: 66 - DNA, 180 bp, Homo sapiens
gtttaagctgcgcaaaagataatttgggcgttggattttgtgttttgttttgcaatgcttaaaagttttttttgattgcggaatttttatatttttgatattagcgcgctttatttgtaataagggtgcgtgaaaggctagtagaggagagagagatggcttatggtgcttaagtttttg

SEQ ID NO: 67 - DNA, 26 bp, Homo sapiens
aaagtgcgtgggaataatgtttattt

SEQ ID NO: 68 - DNA, 24 bp, Homo sapiens
aaacccgcaacctaaaacacaaaa

SEQ ID NO: 69 - DNA, 180 bp, Homo sapiens
catgaacaacctctacagtccctacaaggagatgcccggcatgagccagagcctgtccccgctggccgccacgccgctgggcaacgggctaggcggcctccacaacgcgcagcagagtctgcccaactacggtccgccgggccacgacaaaatgctcagccccaacttcgacgcgcacca

SEQ ID NO: 70 - DNA, 180 bp, Homo sapiens
attatgcgcagctttaattttgatttgtaaaatagcattgggctgcttggcattaatttgtttgagatgatgcgcaatatttttggcggattgggcaatgggttgctgcattgctggttgcttttgtttgagattgagtatggcttgtagaggaatatttttgatatttttaataagtat

SEQ ID NO: 71 - DNA, 25 bp, Homo sapiens
gagatgttcggtatgagttagagtt

SEQ ID NO: 72 - DNA, 28 bp, Homo sapiens
acgaaccgtaattaaacaaactctacta

SEQ ID NO: 73 - DNA, 120 bp, Homo sapiens
agcatggcccacgcgcacgggctgccgtcgcacatgggctgcatgagcgacgtggacgccgacccgcgggacctggaggcattcgccgagcgcttcaagcagcgacgcatcaagctgggg

SEQ ID NO: 74 - DNA, 120 bp, Homo sapiens
ggggttgaattatgcagcgatgaattttgcgagctgcttatggaggtttagggcgcttagctgcaggtgcagcgagtatgttgggtatatgctgctgttgggcatgcgcatttggtatga

SEQ ID NO: 75 - DNA, 23 bp, Homo sapiens
ggttgtcgtcgtatatgggttgt

SEQ ID NO: 76 - DNA, 27 bp, Homo sapiens
cccaacttaatacgtcgctacttaaaa

SEQ ID NO: 77 - DNA, 180 bp, Homo sapiens
attcgccgttttcccccacaaccggcacctgccctggcccagagcgcagcgtccacctgtaccaccgctagatgaagagtacctcaccgcgcgctgcctgggcgcacagctggtgtgccgaccaccgctagatgaagagtacctcaccgcgcgctgcctgggcgcacagctggtgtgccg

SEQ ID NO: 78 - DNA, 180 bp, Homo sapiens
gctttttggggctttttgcatttggcttttgtgggaatttaggttttttttattgcttgcgctgtgtggttgatatgcgggtttgttgcgcgctattttatgagaagtagattgctattatgtttatttgcgatgcgagatttggttttgtttatggctaatatttttttttgctgctta

SEQ ID NO: 79 - DNA, 26 bp, Homo sapiens
taatcggtatttgttttggtttagag

SEQ ID NO: 80 - DNA, 21 bp, Homo sapiens
aacgcgacacaccaactatac

SEQ ID NO: 81 - DNA, 180 bp, Homo sapiens
tggtgccacagcgtccccatctcagacgggcacatcctggagacccggagcgcagatcctccgctccccggcgccgtccaggcgaacaagagaccgtctcagtcccctagaggcccgcagcggggccggtggcgagggagcgcccggctaggggcccgagcagcccgggcgccctgctcc

SEQ ID NO: 82 - DNA, 180 bp, Homo sapiens
ttttgttttgcgggcttgatgagcttggggattggcttgcgagggagcggtggctggggcgatgcttggagattttttgattttgctagagaataagcggatttgctgcggctttttgctttttagatgcgaggcttagaggttttatatgggcagattttatttttgcgatattgtggt

SEQ ID NO: 83 - DNA, 28 bp, Homo sapiens
tattttagacgggtatattttggagatt

SEQ ID NO: 84 - DNA, 26 bp, Homo sapiens
ccgctacgaacctctaaaaaactaaa

(19) United States
(12) Patent Application Publication
(10) Pub. No.: US 2015/0299809 A1
(43) Pub. Date: Oct. 22, 2015

(54) Biomarkers for Clinical Cancer Management

(71) Applicants: AARHUS UNIVERSITET, Aarhus C (DK); REGION MIDTJYLLAND, Viborg (DK)

(72) Inventors: Lise Lotte HANSEN, Risskov (DK); Jens OVERGAARD, Aarhus C (DK); Tomasz Kazimierz WOJDACZ, Aarhus C (DK)

(21) Appl. No.: 14/648,985

(22) PCT Filed: Dec. 5, 2013
(86) PCT No.: PCT/DK2013/050417

§ 371 Date: Jun. 2, 2015

(30) Foreign Application Priority Data
Dec. 5, 2012 (DK) PA 2012 70757

(51) Int. Cl. C12Q 1/68; A61N 5/10; A61B 10/00

(52) U.S. Cl. CPC C12Q 1/6886; A61B 10/0041; A61N 5/10; C12Q 2600/154

(57) ABSTRACT

A method is provided for determining breast cancer, a predisposition to breast cancer, or the prognosis of a breast cancer in a subject, said method comprising in a sample from said subject determining the methylation status of at least one gene locus including regulatory sequences of said gene locus, wherein said genelocusis selected from the group consisting of PHOX2B, FLJ3247, GHSR, HOXB13, HTR1B, ONECUT, POU4F, WT1, LHX1, BC008699, BX161496, CA10, NR2E1, SIX6, SLC38A4, TITF, TMTM132D, CRH, NKX2-3 and HMX.

### FIELD OF INVENTION

The present invention relates to methylation biomarkers for breast cancer.

### BACKGROUND OF INVENTION

The breast cancer incidence has increased since mid-1980s and despite the fact that early detection combined with improvement of treatment has significantly improved survival of cancer patients, the disease still presents a significant problem for the healthcare systems. Breast cancer as well as any other cancer care can be approached at two levels. Firstly, early detection, which is critical for long term survival of the patients, and secondly personalized patient care, which potentially can become the most successful approach for the cancer treatment. Both of the above approaches require biomarkers for patient identification and stratification.

Methylation is a well-established epigenetic process of gene expression regulation. In general terms, methylation of promoter sequences of protein coding genes results in transcriptional down regulation of the gene and hypomethylation of previously methylated promoter regions permits transcription. Two adverse phenomena characterize the process of carcinogenesis: locus specific hypermethylation and global depletion of methyl groups from cancer genomes. Hypermethylation of promoters was widely shown to contribute to silencing of tumour suppressor genes during carcinogenesis. Global hypomethylation of the cancer genome was initially shown to cause genome wide allelic instability but recently the involvement of this process in transcriptional gene regulation is increasingly recognized. DNA methylation changes have been shown to take part in the very first steps of neoplastic transformation which makes methylation biomarkers very attractive target for early cancer detection. Moreover many phenotypic features of the cancer are a consequence of methylation changes. Those changes are predominantly cancer type specific and therefore, have a potential to be powerful biomarkers for cancer patient stratification.

In general, clinically useful biomarker has to show applicability in one of the clinical disease management areas: diagnostics, prognostication and treatment monitoring. More than three decades of epigenetic research have provided a strong research evidence that methylation based biomarkers can be applied in all the above areas of clinical use. Nevertheless, current use of methylation biomarkers in clinical cancer management is very limited. The difficulties in clinical implementation of the methylation biomarkers can be mainly attributed to the lack of clinically validated methylation biomarkers.

### SUMMARY OF INVENTION

The invention relates to methylation biomarkers for breast cancer. The invention provides a number of methylation markers, which can be used to distinguish between breast tumour tissue and healthy tissue. A plurality of individual methylation biomarkers are identified, which show high sensitivity and specificity.

In one aspect, the invention relates to a method of determining breast cancer, a predisposition to breast cancer, the prognosis of a breast cancer, and/or monitoring a breast cancer in a subject, said method comprising in a sample from said subject determining the methylation status of at least one gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).

In another aspect, the invention provides a method for categorizing or staging a breast cancer, or predicting the clinical outcome of a breast cancer, monitoring a treatment of a breast cancer, and monitoring relapse of a breast cancer, of a subject, said method comprising in a sample from said subject determining the methylation status of at least one gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).

Another aspect of the invention pertains to a method of assessing whether a human subject is likely to develop breast cancer, said method comprising

i) providing a sample from said human subject,
ii) determining in said sample the methylation status of at least one gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61), and
iii) on the basis of said methylation status identifying a human subject that is more likely to develop breast cancer.

Another aspect of the invention pertains to a method of evaluating the risk for a subject of contracting cancer, said method comprising in a sample from said subject determining the methylation status of a gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).

In yet another aspect, the present invention relates to a method of treating a breast cancer, said method comprising determining said breast cancer, categorizing or staging said breast cancer, or predicting the clinical outcome of said breast cancer, of a human subject, by a method of the present invention as defined herein, and subsequently providing an appropriate treatment of said breast cancer based on the determination, category, stage or predicted clinical outcome of said breast cancer.

In the methods of the invention, the sample may be a breast tissue sample, or a bodily fluid such as blood or plasma (for example peripheral blood), and methylation status may be determined by any method selected from the group consisting of Methylation-Specific PCR (MSP), Whole genome bisulfite sequencing (BS-Seq), HELP assays, ChIP-on-chip assays, Restriction landmark genomic scanning, Methylated DNA immunoprecipitation (MeDIP), Pyrosequencing of bisulfite treated DNA, Molecular break light assays, and Methyl Sensitive Southern Blotting. In one embodiment, the methylation status is determined by a method comprising the steps of

i) providing a breast tissue sample from said subject comprising nucleic acid material comprising said gene,
ii) processing said nucleic acid sequence using one or more methylation-sensitive restriction endonuclease enzymes,
iii) optionally, amplifying said processed nucleic acid sequence in order to obtain an amplification product, and
iv) analyzing said processed nucleic acid sequence or said amplification product for the presence of processed and/or un processed nucleic acid sequences, thereby inferring the presence of methylated and/or unmethylated nucleic acid sequences.

In a preferred embodiment of the methods, the methylation status is determined by a method comprising the steps of

i) providing a breast tissue sample from said subject comprising nucleic acid material comprising said gene,
ii) modifying said nucleic acid using an agent which modifies unmethylated cytosine or cleaves nucleic acid sequences in a methylation-dependent manner,
iii) amplifying at least one portion of said gene using primers, which span or comprise at least one CpG dinucleotide in said gene in order to obtain an amplification product, and
iv) analyzing said amplification product for the presence of modified and/or unmodified cytosine residues, wherein the presence of modified cytosine residues are indicative of methylated cytosine residues.

The amplified CpG-containing nucleic acid is preferably analyzed by melting curve analysis

However, methylation status may also be determined by methylation specific PCR, bisulfite sequencing, COBRA, endonucleolytic digestion, or DNA methylation arrays.

In a further aspect, the invention provides a kit for determining breast cancer, predisposition to breast cancer, or categorizing or predicting the clinical outcome of a breast cancer, said kit comprising in a package

i. an agent that (a) modifies methylated cytosine residues but not non-methylated cytosine residues; or (b) modifies non-methylated cytosine residues but not methylated cytosine residues; or (c) modifies a nucleic acid sequence in a methylation-dependent manner,
ii. and at least one pair of oligonucleotide primers that specifically hybridizes under amplification conditions to a region of a gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81), such as at least one primer selected from the group consisting of SEQ ID NO: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83 and 84.

In another aspect, the invention provides a use of oligonucleotide primers comprising a sequence, which is a subsequence of a gene loci selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81) or the complement thereof for diagnosing breast cancer in a method of the invention.

In yet another embodiment, the invention provides a method of identifying a therapeutically effective agent for treatment of breast cancer, said method comprising

i. providing a breast cancer cell line comprising one or more genetic loci selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81),
ii. providing one or more potential therapeutic agents,
iii. treating said breast cancer cells by bringing said agents in contact with said breast cancer cells,
iv. determining methylation status of said one or more genetic loci
v. comparing said methylation status of said treated breast cancer cells with the methylation status of said breast cancer cells, when untreated, wherein a decreased level of methylation positive alleles is indicative of a therapeutic agent.

### DESCRIPTION OF DRAWINGS

FIG. 1: Examples of the classes of MS-HRM profiles observed in the case sample material

Standards: 100% methylated—red, 10% methylated (in the background of unmethylated template)—blue, 1% methylated—yellow and unmethylated standard—green. Sample with a representative HRM profile—black. Panel A—fully methylated sample (BC008699 assay), panel B—sample displaying presence of both methylated and unmethylated alleles (HOXB13 assay), panel C—heterogeneously methylated sample (SIX6 assay), panel D—sample with no signs of methylation (CA10 assay).

FIG. 2: Example of low-level methylation sample at HOXB13 DMR

Standards: 100% methylated—red, 10% methylated (in the background of unmethylated template)—blue, 1% methylated—yellow and unmethylated standard—green. Sample with a representative HRM profile—black. The panel illustrates how even small aberrations of the HRM profile from the unmethylated standard represent low-methylation levels (top panel). MS-HRM result with the sample in black and confirmation of the HRM results with sequencing where double both alleles (asterisks) at CpG sites are present (T—unmethylated allele and C—for methylated allele). Lower panel display the same data for an unmethylated sample.

FIG. 3: Examples of the overall methylation screening results in cases and controls and illustrating a shift in methylation of the locus during carcinogenesis.

Standards: 100% methylated—red, 10% methylated (in the background of unmethylated template)—blue, 1% methylated—yellow and unmethylated standard—green. Each panel displays 20 MS-HRM scans for the BC008699 assay in panels A, B and the SIX6 assay in panels C, D. Scans form reference samples are shown in panels A and C. Panels B and D show HRM scans of the cancer samples.

### DETAILED DESCRIPTION OF THE INVENTION

The present invention relates to methylation biomarkers for use in the diagnosis and treatment of breast cancer. Generally, the methylation markers of the invention can be used in methods for identifying subjects, which are predisposed to breast cancer; i.e. subjects having an increased likelihood of developing breast cancer. The methylation markers of the invention can also be used in methods for identifying subjects having breast cancer, and in this case, the markers allow early diagnosis, Further, the markers of the invention provide prognostic information with respect to breast cancer, and this, the markers can be used to identify a subject having breast cancer, and the cancer DNA can be tested for predictive prognostic information based on the methylation markers of the invention, as well as information on which curative and/or ameliorative treatment to provide for the breast cancer. The methylation status of the methylation markers of the invention may also be used to monitor a treatment provided for the curing and/or ameliorating a breast cancer. Additionally, the marker methylation status can be used to monitor relapse of breast cancer for subject previously cured for breast cancer.

Thus, aspects of the present invention relates to i) methods for identifying subjects, which are predisposed to breast cancer, and/or which have a breast cancer, including early stages, such as asymptomatic stages of breast cancer, ii) methods for providing prognostic information of a breast cancer and/or inferring a suitable treatment based thereupon, iii) methods of monitoring a treatment of a breast cancer, and/or monitoring relapse of a breast cancer.

In order to facilitate the understanding of the invention a number of definitions are provided below.

### DEFINITIONS

Amplification according to the present invention is the process wherein a plurality of exact copies of one or more gene loci or gene portions (template) is synthesised. In one preferred embodiment of the present invention, amplification of a template comprises the process wherein a template is copied by a nucleic acid polymerase or polymerase homologue, for example a DNA polymerase or an RNA polymerase. For example, templates may be amplified using reverse transcription, the polymerase chain reaction (PCR), ligase chain reaction (LCR), in vivo amplification of cloned DNA, isothermal amplification techniques, and other similar procedures capable of generating a complementing nucleic acid sequence.

Amplified copies of a targeted genetic region are sometimes referred to as an amplicon.

The term “PCR bias” as used herein refers to conditions, wherein PCR more efficiently amplifies a specific nucleic acid allele. It has been reported that at least some unmethylated nucleic acid templates are more efficiently amplified than methylated nucleic acid template.

A double stranded nucleic acid contains two strands that are complementary in sequence and capable of hybridizing to one another. In general, a gene is defined in terms of its coding strand, but in the context of the present invention, an oligonucleotide primer, which hybridize to a gene as defined by the sequence of its coding strand, also comprise oligonucleotide primers, which hybridize to the complement thereof.

A nucleotide is herein defined as a monomer of RNA or DNA. A nucleotide is a ribose or a deoxyribose ring attached to both a base and a phosphate group. Both mono-, di-, and tri-phosphate nucleosides are referred to as nucleotides.

The term oligonucleotide comprises oligonucleotides of both natural and/or non-natural nucleotides, including any combination thereof. The natural and/or non-natural nucleotides may be linked by natural phosphodiester bonds or by non-natural bonds. Preferred oligonucleotides comprise only natural nucleotides linked by phosphodiester bonds. The oligomer or polymer sequences of the present invention are formed from the chemical or enzymatic addition of monomer subunits. The term “oligonucleotide” as used herein includes linear oligomers of natural or modified monomers or linkages, including deoxyribonucleotides, ribonucleotides, anomeric forms thereof, peptide nucleic acid monomers (PNAs), locked nucleotide acid monomers (LNA), and the like, capable of specifically binding to a single stranded polynucleotide tag by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG,” it will be understood that the nucleotides are in 5′→3′ order from left to right and the “A” denotes deoxyadenosine, “C” denotes deoxycytidine, “G” denotes deoxyguanosine, and “T” denotes thymidine, unless otherwise noted. Usually oligonucleotides of the invention comprise the four natural nucleotides; however, they may also comprise methylated or non-natural nucleotide analogs.

The term “dinucleotide” as used herein refers to two sequential nucleotides. The dinucleotide may be comprised in an oligonucleotide or a nucleic acid sequence. In particular, the dinucleotide CpG, which denotes a cytosine linked to a guanine by a phosphodiester bond, may be comprised in an oligonucleotide according to the present invention, and also comprised in a targeted gene locus sequence according to the present invention. A CpG dinucleotide is also herein referred to as a CpG site. CpG sites are targets for methylation of the cytosine residue.

Methylation status: the term “methylation status” as used herein, refers to the presence or absence of methylation in a specific nucleic acid region. In particular, the present invention relates to detection of methylated cytosine (5-methylcytosine). A nucleic acid sequence, e.g. a gene locus of the invention, may comprise one or more CpG methylation sites. The nucleic acid sequence of the gene locus may be methylated on all methylation sites (i.e. 100% methylated), or unmethylated on all methylation sites (i.e. 0% methylated). However, the nucleic acid sequence may also be methylated on a subset of its potential methylation sites (CpG-sites). In this latter case, the nucleic acid molecule is heterogeneously methylated.

The gene loci methylation markers of the present invention can be used to infer breast cancer based on the relative amount of methylation positive (fully methylated) and methylation negative (fully unmethylated) alleles in a sample comprising in a mixture of nucleic acid molecules from a subject. For example, the methylation status of a specific gene locus marker of the present invention may be that at least 50%, such as on at least 60%, such as on at least 70%, for example on at least 80%, such as on at least 90%, such as on at least 95%, for example on at least 99%, such as least 99.9% of the nucleic acid sequence molecules (alleles) in a sample are methylation positive (fully methylated).

### Method of Determining Breast Cancer

The present invention provides a number of methods for analysing a human subject with respect to breast cancer. In particular, the invention provides methods for determining breast cancer in a human subject, methods for determining a predisposition to breast cancer for a human subject, methods for determining the prognosis of a breast cancer in a subject and/or inferring a suitable treatment, methods for categorizing or staging a breast cancer of a human subject, methods for monitoring a breast cancer, such as monitoring the treatment of a breast cancer and/or relapse of a breast cancer. The methylation biomarkers for breast cancer are described in more detailed herein below. Generally, the one or more methylation biomarkers for breast cancer according to the methods of the invention are selected from a gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).

Thus in one aspect, a method is provided for determining breast cancer, a predisposition to breast cancer, the prognosis of a breast cancer, and/or monitoring a breast cancer in a subject, said method comprising in a sample from said subject determining the methylation status of at least one gene including regulatory sequences of said gene, wherein said gene locus is selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).

In another aspect, a method is provided for categorizing or predicting the clinical outcome of a breast cancer of a subject, said method comprising in a sample from said subject determining the methylation status of at least one gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).

In another aspect, a method is provided for evaluating the risk for a human subject of developing breast cancer, or for monitoring relapse of a breast cancer, said method comprising in a sample from said subject determining the methylation status of a gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).

The invention also in one aspect relates to a method for assessing whether a human subject is likely to develop breast cancer, said method comprising

i) providing a sample from said human subject,
ii) determining in said sample the methylation status of at least one gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61),
iii) on the basis of said methylation status identifying a human subject that is more likely to develop breast cancer.

The methods of the present invention, thus involve determining the methylation status of one or more gene loci as defined herein. Thus, methylation status may be determined for multiple gene loci, for example methylation status for at least two gene loci are determined, such as at least three gene loci, such as at least four gene loci, or five or more gene loci. The plurality of gene loci is preferably selected from a marker gene loci of the invention, i.e. a gene loci selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61).

Generally, increased levels of methylation positive alleles of the respective marker gene locus relative to methylation levels of a predetermined control sample of non-cancer cells is indicative of the presence of a breast cancer, higher likelihood of developing cancer, decreased overall survival, negative outcome, different stage cancer, different grade cancer, and/or higher risk of contracting cancer.

Thus, the methods of the invention preferably comprises the steps of comparing the methylation status of the respective gene locus determined for a subject with a predetermined methylation status for the corresponding gene of a reference sample comprising non-cancer cells, and/or comprising a different stage cancer cells. The predetermined status is preferably determined from non-cancer cells of other subjects, which do not have breast cancer and/or are not predisposed to breast cancer.

The predetermined methylation status differs between the different methylation markers of the invention; cf. tables 0, 3 and 4. For example, for loci ID TITF1, any level of methylation positive alleles above 0%, (table 1, column 4) and in particular above 65.3% (table 1, column 6; i.e. total frequencies of methylation positive alleles) is indicative of a breast cancer, higher likelihood of developing cancer, decreased overall survival, negative outcome, different stage cancer, different grade cancer, and/or higher risk of contracting cancer for a human subject. The same applies to the other genetic loci listed in table 1 below.

TABLE 1

| | | 3 | 4 | 5 | 6 |
|---|---|---|---|---|---|
| | | Methylation | Methylation | Sum of methylation | Methylation |
| | 2 | positive | positive | positive and low | positive |
| | SEQ | frequency (%) | frequency (%) | methylation frequency | frequency (%) |
| 1 | ID | for non- | indicative of | (%) for non-cancer | indicative of |
| Loci ID | NO: | cancer subjects | breast cancer | subjects | breast cancer |
| TITF1 | 1 | 0 | >0 | 65.3 | >65.3 |
| HOXB13 | 5 | 0 | >0 | 18.1 | >18.1 |
| NR2E1 | 9 | 0 | >0 | 8.3 | >8.3 |
| HTR1B | 13 | 0 | >0 | 0 | >0 |
| HMX2 | 17 | 0 | >0 | 65.2 | >65.2 |
| BC008699 | 21 | 4.2 | >4.2 | 33.3 | >33.3 |
| SLC38A4 | 25 | 4.8 | >4.8 | 53.2 | >53.2 |
| FLJ32447 | 29 | 0 | >0 | 68.6 | >68.6 |
| WT1 | 33 | 0 | >0 | 2.8 | >2.8 |
| TMEM132D | 37 | 0 | >0 | 52.9 | >52.9 |
| NKX2-3 | 41 | 0 | >0 | 100 | 1-100 |
| GHSR | 45 | 0 | >0 | 2.8 | >2.8 |
| ONECUT | 49 | 0 | >0 | 33.8 | >33.8 |
| LHX1 | 53 | 25.0 | >25.0 | 25 | >25 |
| SIX6 | 57 | 0 | >0 | 11.6 | >11.6 |
| CA10 | 61 | 0 | >0 | 51.1 | >51.1 |
| CHR | 65 | 0 | >0 | 100 | 1-100 |
| POU4F | 69 | 0 | >0 | 29.7 | >29.7 |
| PHOX2B | 73 | 0 | >0 | 100 | 1-100 |

Thus, for methylation marker locus, identified as TITF1 a level of methylation positive alleles above 0%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as preferably above 65.3%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and/or relapse of breast cancer, and thus indicates that a given treatment being monitored is inefficientinefficient.

For methylation marker locus, identified as HOXB13, a level of methylation positive alleles above 0%, such as above 5%, such as above 10%, such as above 15%, such as preferably above 18.1%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and/or relapse of breast cancer, and thus indicates that a given treatment being monitored is inefficient.

For methylation marker locus, identified as NR2E1, a level of methylation positive alleles above 0%, such as above 5%, such as preferably above 8.3%, such as above 10%, such as above 15%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and/or relapse of breast cancer, and thus indicates that a given treatment being monitored is inefficient.

For methylation marker locus, identified as HTR1 B, a level of methylation positive alleles above 0%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and/or relapse of breast cancer, and thus indicates that a given treatment being monitored is inefficient.

For methylation marker locus, identified as HMX2, a level of methylation positive alleles above 0%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as preferably above 65.2%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and/or relapse of breast cancer, and thus indicates that a given treatment being monitored is inefficient.

For methylation marker locus, identified as BC008699, a level of methylation positive alleles above 0%, such as preferably above 4.2%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as preferably above 65.2%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and/or relapse of breast cancer, and thus indicates that a given treatment being monitored is inefficient.

For methylation marker locus, identified as SLC38A4, a level of methylation positive alleles above 0%, such as preferably above 4.8%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as preferably above 53.2%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and/or relapse of breast cancer, and thus indicates that a given treatment being monitored is inefficient.

For methylation marker locus, identified as FLJ32447, a level of methylation positive alleles above 0%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as preferably above 68.6%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as WT1, a level of methylation positive alleles above 0%, such as preferably above 2.8%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as TMEM132D, a level of methylation positive alleles above 0%, such as above 5%, such as above 10%, such as above 15%, such as above 20%, such as above 25%, such as above 30%, such as above 35%, such as above 40%, such as above 45%, such as above 50%, such as preferably above 52.9%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as NKX2-3, a level of methylation positive alleles above 0%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as GHSR, a level of methylation positive alleles above 0%, such as preferably above 2.8%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as ONECUT, a level of methylation positive alleles above 0%, such as above 5%, such as above 10%, such as above 15%, such as above 20%, such as above 25%, such as above 30%, such as preferably above 33.8%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as LHX1, a level of methylation positive alleles above 0%, such as above 5%, such as above 10%, such as above 15%, such as above 20%, such as preferably above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as SIX6, a level of methylation positive alleles above 0%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as CA10, a level of methylation positive alleles above 0%, such as above 5%, such as above 10%, such as preferably above 11.6%, such as above 15%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as CHR, a level of methylation positive alleles above 0%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as POU4F, a level of methylation positive alleles above 0%, such as above 5%, such as above 10%, such as above 15%, such as above 20%, such as above 25%, such as preferably above 29.7%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

For methylation marker locus, identified as PHOX2B, a level of methylation positive alleles above 0%, such as above 5%, such as above 10%, such as above 15%, such as above 20%, such as above 25%, such as above 30%, such as above 35%, such as above 40%, such as above 45%, such as above 50%, such as above 55%, such as above 60%, such as above 65%, such as above 70%, such as above 75%, such as above 80%, such as above 85%, such as above 90%, such as above 95%, such as above 96%, 97%, 98%, or 99%, such as 100% is indicative of breast cancer, a predisposition to breast cancer, increased risk of breast cancer, the prognosis of breast cancer, and and/or relapse of breast cancer, and thus indicates that a given treatment being monitored is inefficient.

### Method for Treatment of Breast Cancer

Aspects of the invention also relates to methods for determining the prognosis of a breast cancer in a subject and/or inferring a suitable treatment, as well as for monitoring a breast cancer, and in particular monitoring the treatment of a breast cancer and/or monitoring relapse of a breast cancer.

So in one aspect, a method is provided for treatment of breast cancer in a human subject, the method comprises the steps of

i. determining breast cancer, a predisposition to breast cancer, or the prognosis of a breast cancer in a subject by a method of the present invention, as defined elsewhere herein,
ii. selecting human subjects having breast cancer, a predisposition to breast cancer, or a relapse of a breast cancer,
iii. subjecting said subjects identified in step ii. to a suitable treatment for breast cancer.

The step of determining breast cancer by a method of the present invention allows early detection of breast cancer, and therefore allows treatment of the cancer to be initiated before developing into later stages and/or before forming metastases. This allows the use of less serious types of therapeutic interventions, and may for example avoid the need for surgery, such as surgical removal of the entire breast. In one embodiment, the selected human subject is subjected to a treatment selected form surgery, chemotherapy and/or radiotherapy, however, in a preferred embodiment, the treatment is radiotherapy. In one embodiment, the treatment is a combination of chemotherapy and radiotherapy, for example chemotherapy followed by radiotherapy.

The methylation markers also allow monitoring relapse of breast cancer, as well as offering a personalized treatment of breast cancer by surveillance and quality of control of the treatment offered, thereby allowing terminating ineffective treatments and offering alternative treatments. Thus, in another aspect, the invention provides a method for personalized treatment of a breast cancer of a human subject, said method comprising

i) in a sample from said human subject, determining the methylation status of a gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81)
ii) providing a treatment of breast cancer to said human subject,
iii) after a sufficient amount of time having provided the treatment, in a sample from said human subject, determining the methylation status of said gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81)
iv) comparing the methylation status of said gene locus before and after treatment, and
v) if methylation of said genetic locus is similar to the methylation before treatment, terminating said provided treatment and preferably offiring an alternative treatment, of
vi) if methylation of said genetic locus is reduced relative to the methylation before treatment, continuing said provided treatment.

### Methylation Biomarkers for Breast Cancer

As described herein above, the present invention provides a number of different methods for evaluating breast cancer in a human subject based on methylation status of specific gene loci. The invention also provides specific oligonucleotide primers and kits for use in determining methylation status of specific gene loci, which are methylation biomarkers for breast cancer according to the present invention. These gene loci include BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).

Generally, in the methods of the invention, the methylation status is determined for at least one gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81). In one embodiment, the methylation status is determined for a gene locus selected from the group consisting of FLJ3247, GHSR, HOXB13, HTR1B, ONECUT, POU4F, WT1, LHX1, BX161496, CA10, NR2E1, PHOX2B, SIX6, SLC38A4, TITF, TMTM132D, CRH, NKX2-3 and HMX. In another embodiment, the methylation status is determined for a gene locus selected from the group consisting of FLJ3247, GHSR, ONECUT, POU4F, WT1, LHX1, BX161496, CA10, NR2E1, SIX6, SLC38A4, TITF, TMTM132D and HOXB13. In another embodiment, the methylation status is determined for a gene locus selected from the group consisting of HOXB13, FLJ3247, ONECUT, NR2E1 and TMTM132D.

In one embodiment, the methylation status is determined for a gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).

In one embodiment, the methylation status is determined for a gene locus selected from the group consisting of HOXB13, FLJ3247 and NR2E1. The methylation status may be determined for HOXB13, FLJ3247 and/or NR2E1. In another preferred embodiment, the gene locus is HTR1B.

In one preferred embodiment, methylation status is determined in one or more gene loci selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9) and/or CHR (SEQ ID NO: 49).

In another preferred embodiment, methylation status is determined in one or more gene loci selected from the group consisting of TMEM132D (SEQ ID NO: 81), TITF1 (SEQ ID NO: 37), NR2E1 (SEQ ID NO: 25), CA10 (SEQ ID NO: 5) and/or GHSR (SEQ ID NO: 53).

In one specific embodiment, methylation status is determined in the PHOX2B gene locus, and in a further embodiment, methylation status is determined in the PHOX2B gene locus and at least one additional gene locus selected from the group consisting of POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5) and GHSR (SEQ ID NO: 53).

In another specific embodiment, methylation status is determined in the POU4F gene locus, and in a further embodiment, methylation status is determined in the POU4F gene locus and at least one additional gene locus selected from the group consisting of PHOX2B (SEQ ID NO: 29), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5) and GHSR (SEQ ID NO: 53).

In another specific embodiment, methylation status is determined in the SIX6 gene locus, and in a further embodiment, methylation status is determined in the SIX6 gene locus and at least one additional gene locus selected from the group consisting of PHOX2B (SEQ ID NO: 29), POU4F (SEQ ID NO: 73), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5) and GHSR (SEQ ID NO: 53).

In another specific embodiment, methylation status is determined in the WT1 gene locus, and in a further embodiment, methylation status is determined in the WT1 gene locus and at least one additional gene locus selected from the group consisting of PHOX2B (SEQ ID NO: 29), POU4F (SEQ ID NO: 73), SIX6 (SEQ ID NO: 33), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5) and GHSR (SEQ ID NO: 53).

In one specific embodiment, methylation status is determined in the ONECUT gene locus, and in a further embodiment, methylation status is determined in the ONECUT gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5) and GHSR (SEQ ID NO: 53).

In one specific embodiment, methylation status is determined in the NKX2-3 gene locus, and in a further embodiment, methylation status is determined in the NKX2-3 gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5) and GHSR (SEQ ID NO: 53).

In one specific embodiment, methylation status is determined in the FLJ32447 gene locus, and in a further embodiment, methylation status is determined in the FLJ32447 gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), CHR (SEQ ID NO: 49), TMEM132D (SEQ ID NO: 81), TITF1 (SEQ ID NO: 37), NR2E1 (SEQ ID NO: 25), CA10 (SEQ ID NO: 5) and GHSR (SEQ ID NO: 53).

In one specific embodiment, methylation status is determined in the CHR gene locus, and in a further embodiment, methylation status is determined in the CHR gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), TMEM132D (SEQ ID NO: 81), TITF1 (SEQ ID NO: 37), NR2E1 (SEQ ID NO: 25), CA10 (SEQ ID NO: 5) and GHSR (SEQ ID NO: 53).

In one specific embodiment, methylation status is determined in the TMEM132D gene locus, and in a further embodiment, methylation status is determined in the TMEM132D gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TITF1 (SEQ ID NO: 37), NR2E1 (SEQ ID NO: 25), CA10 (SEQ ID NO: 5) and GHSR (SEQ ID NO: 53).

In another specific embodiment, methylation status is determined in the TITF1 gene locus, and in a further embodiment, methylation status is determined in the TITF1 gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), NR2E1 (SEQ ID NO: 25), CA10 (SEQ ID NO: 5) and GHSR (SEQ ID NO: 53).

In another specific embodiment, methylation status is determined in the NR2E1 gene locus, and in a further embodiment, methylation status is determined in the NR2E1 gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), CA10 (SEQ ID NO: 5) and GHSR (SEQ ID NO: 53).

In another specific embodiment, methylation status is determined in the CA10 gene locus, and in a further embodiment, methylation status is determined in the CA10 gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25) and GHSR (SEQ ID NO: 53).

In another specific embodiment, methylation status is determined in the GHSR gene locus, and in a further embodiment, methylation status is determined in the GHSR gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25) and CA10 (SEQ ID NO: 5).

In one preferred embodiment, methylation status is determined for a gene locus for which the frequency of methylation positive alleles is 0% for normal/noncancer human subjects. Thus, in one embodiment of the methods of the invention, the methylation status is determined for a gene locus selected from the group consisting of CA10 (SEQ ID NO: 5), FLJ32447 (SEQ ID NO: 9), HMX2 (SEQ ID NO: 13), HS3ST2 (SEQ ID NO: 17), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73) and TMEM132D (SEQ ID NO: 81).

In another embodiment, the methylation status is determined for a gene locus for which the frequency of negative methylation is 0% normal/noncancer human subjects, such as gene locus HTR1B (SEQ ID NO: 61).

In another embodiment, methylation status is determined for a gene locus for which the frequency of methylation positive alleles is 0% for normal/noncancer human subjects, and for which the frequency of methylation negative alleles is 0% for breast cancer human subjects. Thus, in one embodiment of the methods of the invention, the methylation status is determined for a gene locus selected from the group consisting of TITF1, SLC38A4, FLJ32447, NKX2-3, ONECUT, LHX1, CHR and PHOX2B.

In yet another preferred embodiment, methylation status is determined for a gene locus for which the frequency of methylation negative alleles is 0% for breast cancer human subjects. Thus, in one embodiment of the methods of the invention, the methylation status is determined for a gene locus selected from the group consisting of TITF1, FLJ32447, NKX2-3, ONECUT, LHX1, CHR and PHOX2B.

DNA sequences of specific gene loci are provided herein below. Thus, in a preferred embodiment, the methylation status is determined in a gene locus identified by SEQ ID NO: 1, 5, 9, 13, 17, 21, 25, 29, 33, 37, 41, 45, 49, 53, 57, 61, 65, 69, 73, 77 and/or 81; cf. table 2.

The corresponding sequences of the same loci after bisulfite modification and amplification are identified by SEQ ID NO: 2, 6, 10, 14, 18, 22, 26, 30, 34, 38, 42, 46, 50, 54, 58, 62, 66, 70, 74, 78 and/or 82, respectively.

In a preferred embodiment, the methylation status is determined by a method comprising amplifying a gene locus of the invention using at least one primer selected from the group consisting of SEQ ID NO: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83 and 84. Methylation status is preferably determined for a gene locus mentioned in table 2 using the respective forward primer and/or reverse primer identified in table 2; i.e.

BC008699: forward primer SEQ ID NO: 3 and/or reverse primer SEQ ID NO: 4;
CA10: forward primer SEQ ID NO: 7 and/or reverse primer SEQ ID NO: 8;
. . . etc; and
TMEM132D: forward primer SEQ ID NO: 83 and/or reverse primer SEQ ID NO: 84.

Thus, in one preferred embodiment, the methylation status is determined in a genetic region of a gene locus of the invention, wherein said region is delineated by the primer pairs identified in table 2 for each respective gene; i.e.

for BC008699: primers SEQ ID NO: 3 and/or 4;
for CA10: primers SEQ ID NO: 7 and/or 8;
. . . etc. . . . ; and
for TMEM132D: primers SEQ ID NO: 83 and/or 84.

TABLE 2
Markers Sequence table

| Marker | Non-modified | Modified | Forward | Reverse |
|---|---|---|---|---|
| gene/Loci | Gene/loci | Gene/loci | primer | primer |
| ID | SEQ ID NO | SEQ ID NO | SEQ ID NO | SEQ ID NO |
| BC008699 | 1 | 2 | 3 | 4 |
| CA10 | 5 | 6 | 7 | 8 |
| FLJ32447 | 9 | 10 | 11 | 12 |
| HMX2 | 13 | 14 | 15 | 16 |
| HS3ST2 | 17 | 18 | 19 | 20 |
| LHX1 | 21 | 22 | 23 | 24 |
| NR2E1 | 25 | 26 | 27 | 28 |
| PHOX2B | 29 | 30 | 31 | 32 |
| SIX6 | 33 | 34 | 35 | 36 |
| TITF1 | 37 | 38 | 39 | 40 |
| WT1 | 41 | 42 | 43 | 44 |
| BX161496 | 45 | 46 | 47 | 48 |
| CHR | 49 | 50 | 51 | 52 |
| GHSR | 53 | 54 | 55 | 56 |
| HOXB13 | 57 | 58 | 59 | 60 |
| HTR1B | 61 | 62 | 63 | 64 |
| NKX2-3 | 65 | 66 | 67 | 68 |
| ONECUT | 69 | 70 | 71 | 72 |
| POU4F | 73 | 74 | 75 | 76 |
| SLC38A4 | 77 | 78 | 79 | 80 |
| TMEM132D | 81 | 82 | 83 | 84 |

### Sample

According to the present invention, the methylation status of one or more gene loci is determined in a sample from a human subject. Thus, the sample of the invention comprises biological material, in particular genetic material comprising nucleic acid molecules. The nucleic acid molecules may be extracted from the sample prior to the analysis. The sample may be obtained or provided from any human source. In one embodiment, determination of methylation status of a gene locus or genetic region of the invention is performed on samples selected from the group consisting of breast tissue, hematopoietic tissue, bone marrow, expiration air, stem cells, including cancer stem cell, and body fluids, such as sputum, urine, blood and sweat.

In preferred embodiments the sample is or comprises breast tissue, such as breast cells and/or genetic material of breast cells.

It is wellknown that tumor DNA may leak to the blood stream or other bodily fluids, so in one preferred embodiment, the sample is a body fluid, such as sputum, urine, blood and sweat. In particular, it is preferred that the sample is a blood or plasma sample. Body fluids are often retrievable by less invasive methods than breast tissue, which must be obtained surgically for example by biopsies.

The provided sample is in one embodiment a formalin-fixed paraffin-embedded (ffpe) sample, for example an ffpe sample, wherein prestages to breast cancer can be seen. In particular, the sample used for predetermining methylation status can be an ffpe sample. Many ffpe samples may be provided, which can give rise to statistically strong predetermined values with respect to evaluation of breast cancer risk, categorizing or staging a breast cancer of a human subject, methods for monitoring a breast cancer, such as monitoring the treatment of a breast cancer and/or relapse of a breast cancer.

The nucleic acid to be analysed for the presence of methylated CpG may be extracted from the samples by a variety of techniques such as that described by Maniatis, et al (Molecular Cloning: A Laboratory Manual, Cold Spring Harbor, N.Y., pp 280, 281, 1982). However, the sample may be used directly.

Any nucleic acid, in purified or nonpurified form, can be utilized as the starting nucleic acid or acids, provided it contains, or is suspected of containing, the specific nucleic acid sequence containing the methylation target site (e.g., CpG). The specific nucleic acid sequence which is to be amplified may be a part of a larger molecule or is present initially as a discrete molecule. The nucleic acid sequence to be amplified need not to be present in a pure form, it may for example be a fraction of a complex mixture of other DNA molecules, and/or RNA. In one example, the nucleic acid sequence is a fraction of a genomic nucleic acid preparation.

Extremely low amounts of nucleic acid may be used as target sequence according to the methods of the present invention. It is appreciated by the person skilled in the art that in practical terms no upper limit for the amount of nucleic acid to be analysed exists. The problem that the skilled person may encounter is that the amount of sample to be analysed is limited. Therefore, it is beneficial that the method of the present invention can be performed on a small amount of sample and thus a limited amount of nucleic acid in said sample. The present methods allow the detection of only very few nucleic acid copies. The amount of the nucleic acid to be analysed is in one embodiment at least 0.01 ng, such as 0.1 ng, such as 0.5 ng, for example 1 ng, such as at least 10 ng, for example at least 25 ng, such as at least 50 ng, for example at least 75 ng, such as at least 100 ng, for example at least 125 ng, such as at least 150 ng, for example at least 200 ng, such as at least 225 ng, for example at least 250 ng, such as at least 275 ng, for example at least 300 ng, 400 ng, for example at least 500 ng, such as at least 600 ng, for example at least 700 ng, such as at least 800, ng, for example at least 900 ng or such as at least 1000 ng.

In one preferred embodiment the amount of nucleic acid as the starting material for the method of the present invention is approximately 50 ng, alternatively 100 ng or 200 ng.

### Methylation Status

The methods of the present invention for determining breast cancer in a human subject, methods for determining a predisposition to breast cancer for a human subject, methods for determining the prognosis of a breast cancer in a subject and/or inferring a suitable (personalized) treatment, methods for categorizing or staging a breast cancer of a human subject and methods for monitoring a breast cancer, all include a step of providing or obtaining a sample from the human subject, and in that sample determining the methylation status of at least one genetic locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81), as well as subregions thereof, in particular the subregions delineated by the respective primer pairs identified in table 3.

Methylation status of the target gene loci or genetic regions of the present invention may be determined by any suitable method available to the skilled person for detecting methylation status. However, in a preferred embodiment, methylation status is determined by a quantitative method, which is capable of detecting levels of methylation positive alleles and/or methylation negative alleles in a population of target molecules present in a sample. For example, the quantitative method is preferably capable of detecting different levels of methylation positive alleles of a given target locus sequence, such as detecting whether 0%, less than 1%, more that 1%, such as approximately 10%, 25%, 50%, 75% or 100% of the alleles of a given marker locus are methylation positive. Some techniques in the art merely detect the presence of one or more methylation positive and/or methylation negative alleles of a given target sequence without providing quantitative data, and without providing information of the relative levels of methylation positive and methylation negative alleles. However, preferred methods of the present invention provide a quantitative measure of the relative level of methylation positive alleles of a specific target region.

The term “methylation status” as used herein, refers to the extent to which a nucleic acid region and/or in particular a CpG methylation site is methylated or unmethylated, which may be expressed as the methylation level of a given sample. The methylation status of a single CpG methylation site can be either methylated or unmethylated. A nucleic acid sequence comprising multiple potential methylation (CpG) sites, may be methylated on only a subset of those CpG sites. Such nucleic acid molecules/alleles are heterogeneous methylated. The term “methylation status”, thus, refers to whether a nucleic acid sequence is methylation positive (methylated on all CpG sites), is methylation negative (all CpG sites of the sequence is unnmethylated), or is heterogeneous methylated (a subset of CpG sites of the sequence is methylated. The methods for inferring breast cancer of the present invention, thus determine methylation status of specific methylation markers by determining whether a specific methylation marker in a sample obtained or provided from a subject is methylation positive, methylation negative or heterogeneously methylated, as well as detecting the relative level of methylated alleles of a given locus. The methods may also include detecting marker sequences with low methylation, which defines methylation of less than 1% of the alleles of a sample.

In one embodiment of methods of the present invention for determining breast cancer in a human subject, for determining a predisposition to breast cancer for a human subject, for determining the prognosis of a breast cancer in a subject and/or inferring a suitable treatment, for categorizing or staging a breast cancer of a human subject, and/or for monitoring a breast cancer, such as monitoring the treatment of a breast cancer and/or relapse of a breast cancer, the methylation status is determined by use of methylation-sensitive restriction enzymes. Many restriction enzymes are sensitive to the DNA methylation states. Cleavage can be blocked or impaired when a particular base in the recognition site is modified. For example, the MspJl family of restriction enzymes has been found to be dependent on methylation and hydroxymethylation for cleavage to occur. These enzymes excise ˜32 base pair fragments containing a centrally located 5-hmC or 5-mC modified residue that can be extracted and sequenced. Due to the known position of this epigenetic modification, bisulfite conversion is not required prior to downstream analysis.

Methylation-sensitive enzymes are well-known in the art and include:

### AatII, AccII, Aor13HI, Aor51HI, BspT104I, BssHII, Cfr10I, ClaI CpoI, Eco52I, HaeII, HapII, HhaI, MluI, NaeI, NotI, NruI, NsbI, PmaCI, Psp1406I, PvuI, SacII, SaII, SmaI and SnaBI.

The digested nucleic acid sample is subsequently analysed by for example gel electrophoresis.

So, in one embodiment of the methods of the invention, methylation status is determined by a method comprising the steps of

i) providing a sample, such as a breast tissue sample or a blood or plasma sample from said subject comprising nucleic acid material comprising said gene,
ii) processing said nucleic acid sequence using one or more methylation-sensitive restriction endonuclease enzymes,
iii) optionally, amplifying said processed nucleic acid sequence in order to obtain an amplification product, and
iv) analyzing said processed nucleic acid sequence or said amplification product for the presence of processed and/or unprocessed nucleic acid sequences, thereby inferring the presence of methylated and/or unmethylated nucleic acid sequences.

In a preferred embodiment of methods of the present invention, the methodology employed for determining methylation status is determined by a method, which comprises at least the steps of modifying the DNA with an agent which targets either methylated or unmethylated sequences, amplifying the DNA, and analysing the amplification products.

For example, amplification product is analysed by detecting the presence or absence of amplification product, wherein the presence of amplification product indicates that the target nucleic acid has not been cleaved by the restriction enzymes, and wherein the absence of amplification product indicates that the target nucleic acid has been cleaved by the restriction enzymes.

Thus, generally, the in the methods of the invention methylation status is determined by a method comprising the steps of

i) providing a sample, such as a breast tissue sample or a blood or plasma sample from said subject comprising nucleic acid material comprising a gene locus of the invention,
ii) modifying said nucleic acid material using an agent, which modifies nucleic acid sequences in a methylation-dependent manner,
iii) amplifying at least one portion of said gene locus using primers, which span or comprise at least one CpG dinucleotide in said gene locus in order to obtain an amplification product, and
iv) analyzing said amplification product for the presence of modified and/or unmodified cytosine residues, wherein the presence of modified cytosine residues are indicative of methylated cytosine residues.

For example, the method comprises the steps of

i) providing a sample, such as a breast tissue sample, from said subject comprising nucleic acid material comprising said gene locus,
ii) modifying said nucleic acid using an agent which modifies unmethylated cytosine,
iii) amplifying at least one portion of said gene locus using primers, which span or comprise at least one CpG dinucleotide in said gene locus in order to obtain an amplification product, and
iv) analyzing said amplification product.

The amplification product can be analysed for nucleic acid substitutions resulting from conversion of modified cytosine residues, preferably wherein the presence of converted cytosine residues are indicative of unmethylated cytosine residues, and presence of unconverted cytosine residues is indicative of methylated cytosine residues. Typically, unmethylated cytosine is converted to thymidine after bisulphite treatment and amplification, while methylated cytosine is left unchanged after same treatment.

In a preferred embodiment, the amplification product is analysed by melting curve analysis; cf. herein below.

The amplification product, the amplicon, is in a preferred embodiment a genetic region of a gene of the invention, wherein said region is delineated by the primer pairs identified in table 2 for each respective gene; i.e.

for BC008699: primers SEQ ID NO: 3 and 4;
for CA10: primers SEQ ID NO: 7 and 8;
. . . etc. . . . ; and
for TMEM132D: primers SEQ ID NO: 83 and 84.

### Modification of DNA

The method for determining methylation status in the present invention preferably comprise a step of modifying the nucleic acids comprised in the sample, or extracted from the sample, using an agent which specifically modifies unmethylated cytosine in the nucleic acid. As used herein the term “modifies” refers the specific modification of either an unmethylated cytosine or a methylated cytosine, for example the specific conversion of an unmethylated cytosine to another nucleotide which will distinguish the modified unmethylated cytosine from a methylated cytosine. In one preferred embodiment, an agent modifies unmethylated cytosine to uracil. Such an agent may be any agent conferring said conversion, wherein unmethylated cytosine is modified, but not methylated cytosine. In one preferred embodiment the agent for modifying unmethylated cytosine is sodium bisulfite. Sodium bisulfite (NaHSO₃) reacts readily with the 5,6-double bond of cytosine, but only poorly with methylated cytosine. The cytosine reacts with the bisulfite ion, forming a reaction intermediate in the form of a sulfonated cytosine which is prone to deamination, eventually resulting in a sulfonated uracil. Uracil can subsequently be formed under alkaline conditions which removes the sulfonate group.

During a nucleic acid amplification process, uracil will by the Taq polymerase be recognised as a thymidine. The product upon PCR amplification of a Sodium bisulfite modified nucleic acid contains cytosine at the position where a methylated cytosine (5-methylcytosine) occurred in the starting template DNA of the sample. Moreover, the product upon PCR amplification of a Sodium bisulfite modified nucleic acid contains thymidine at the position where an unmethylated cytosine (5-methylcytosine) occurred in the starting template DNA of the sample. Thus, an unmethylated cytosine is converted into a thymidine residue upon amplification of a bisulfite modified nucleic acid.

In a preferred embodiment of the present invention, the nucleic acids are modified using an agent which modifies unmethylated cytosine in the nucleic acid. In a specific embodiment, such an agent is a bisulfite, hydrogen sulfite, and/or disulfite reagent, for example sodium bisulfite.

However, in another embodiment, an agent is used, which specifically modifies methylated cytosine in the nucleic acid and does not modify unmethylated cytosine.

### Amplifying Step

After modification of the nucleic acids of the sample, the specific genetic region selected for determination of methylation status is preferably amplified in order to generate and thereby obtain multiple copies (amplicons) of the respective genetic regions, which can allow its further analysis with respect to methylation status. The amplification is preferably preformed using at least one oligonucleotide primer, which targets the specific genetic region comprising methylation markers for breast cancer according to the present invention. Most preferably amplification is performed using two oligonucleotide primers, which delineates the analysed region. The skilled person may use his common general knowledge in designing suitable primers. However, in a preferred embodiment, at least one, and preferably two methylation-independent oligonucleotide primers are employed for amplification of the modified nucleic acid. The nature of methylation-independent primers is described on more detail herein below.

The amplifying step is a polymerisation reaction wherein an agent for polymerisation is involved, effecting an oligonucleotide primer extension. The agent for polymerization may be any compound or system which will function to accomplish the synthesis of primer extension products, including enzymes. Enzymes that are suitable for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation also known as Tag polymerases). Suitable enzymes will facilitate combination of the nucleotides in the proper manner to form the primer extension products which are complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

A preferred method for amplifying the modified nucleic acid by means of at least one methylation-independent oligonucleotide primer is by the polymerase chain reaction (PCR), as described herein and as is commonly used by those skilled in the art. It is appreciated that PCR amplification requires a set of oligonucleotide primers, one forward primer and one reverse primer. According to the present invention, the forward primer is a methylation independent primer. The reverse primer is in another embodiment a methylation independent primer. However, both reverse and forward primer may be methylation independent oligonucleotide primers according to the definitions herein.

The amplification product (amplicon) may be of any length, however in one preferred embodiment, the amplification product comprise between 15 and 1000 nucleotides, such as between 15 and 500 nucleotides, such as between 50 and 120 nucleotides, preferably between 80 and 100 nucleotides. In a preferred embodiment, the amplicon is delineated by the primers identified in table 2 for each respective gene, cf. herein above.

The PCR reaction is characterised by three steps a) melting a nucleic acid template, b) annealing at least one methylation-independent oligonucleotide primer to said nucleic acid template, and c) elongating said at least one methylation-independent oligonucleotide primer.

### Melting

The melting of a CpG-containing nucleic acid template may also be referred to as strand separation. Melting is necessary where the target nucleic acid contains two complementary strands bound together by hydrogen bonds. This strand separation can be accomplished using various suitable denaturing conditions, including physical, chemical, or enzymatic means. One physical method of separating nucleic acid strands involves heating the nucleic acid until it is denatured. The denaturation by heating is the preferred procedure for melting in the present invention. Heat denaturation involves temperatures ranging from about 60 degrees Celsius to 100 degrees Celsius. The time for melting may be in the range of 5 seconds to 10 minutes or even longer for initial melting of the template.

The melting temperature is typically between 80 and 90 degrees Celsius, such as at least 81, for example at least 82, such as at least 84, preferably at least 85, at least 86, such as at least 87, for example at least 88 degrees Celsius. The PCR reaction mixture is incubated at the melting temperature for at least 5 seconds, alternatively at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 seconds.

### Annealing

Separated strands are used as a template for the synthesis of additional nucleic acid strands. It is understood that the separated strands may result from the separation of complementary strands in an originally double stranded nucleic acid. However, separated strands originally single stranded are also used as templates according to the present invention. The synthesis of additional nucleic acid strands is performed under conditions that allow the hybridisation of oligonucleotide primers to templates. Such a step is herein referred to as annealing. The oligonucleotide primers form hydrogen bonds with the template.

The annealing temperature is between 40 and 75 degrees Celsius, such as at least 40, at least 45, for example at least 50, at least 52, at least 54, at least 56, at least 57, at least 58, at least 59 preferably at least 60, at least 61, at least 62, at least 63, at least 64, at least 65, at least 66, at least 67, for example at least 68, at least 69, at least 70, at least 72, at least 73, at least 75 degrees Celsius. The PCR reaction mixture is incubated at the annealing temperature for 1 to 100 seconds, such as at least 1, at least 2, at least 3, at least 4, preferably at least 5, at least 6, at least 7, at least 8, at least 9, at least 10, alternatively at least 11, at least 13, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 seconds.

In a specific embodiment of the present invention, the annealing temperature is between at least 15 degrees Celsius above the optimal annealing temperature, such as at least 1, at least 2, at least 3, at least 4, at least 5, at least 6, at least 7, at least 8, at least 9, at least 10, at least 11, at least 12, at least 13, at least 14, at least 15 degrees Celsius above the optimal annealing temperature.

The optimal annealing temperature can be calculated by standard algorithms, as known to people skilled within the art. In one embodiment, the optimal primer annealing temperature (Tm) is calculated as: Tm=4(G+C)+2(A+T), wherein G, C, A, T designates the number of the respective nucleotides. In another embodiment, the optimal primer annealing temperature (Tm) is calculated as:

Tm=64.9° C.+41° C.×(number of G's and C's in the primer−16.4)/N, where N is the length of the primer. However, the annealing temperature should be empirically determined in respect of each specific primer. The modulation of the annealing temperature is used to adjust hybridization stringency as described elsewhere herein. Thus, the optimal annealing temperature should be set at a level, wherein the PCR bias towards amplification of unmethylated nucleic acid template is balanced by the less efficient annealing of methylation-independent oligonucleotide primer according to the present invention to unmethylated nucleic acid target sequence.

Generally, the choice of annealing temperature depends on the sensitivity of the assay, and the composition of the sample with respect to the relative levels of methylation positive and methylation negative alleles. Thus, optimal annealing temperatures should preferably be determined for each sample. However, in one embodiment, the annealing temperature in respect of specific methylation-independent oligonucleotide primer according to the present invention is as specified in table 3 below.

TABLE 3

| | | | | Cycling: |
|---|---|---|---|---|
| | | | Annealing | melting, |
| Marker | Forward | Reverse | temp, | annealing, |
| gene/Loci | primer | primer | (degrees | elongation |
| ID | SEQ ID NO | SEQ ID NO | Celsius) | (seconds) |
| BC008699 | 3 | 4 | 65 | 15, 15, 20 |
| CA10 | 7 | 8 | 63 | 10, 10, 15 |
| FLJ32447 | 11 | 12 | 61 | 20, 20, 30 |
| HMX2 | 15 | 16 | 66 | 15, 15, 20 |
| HS3ST2 | 19 | 20 | 63 | 10, 10, 15 |
| LHX1 | 23 | 24 | 63 | 10, 10, 15 |
| NR2E1 | 27 | 28 | 65 | 10, 10, 15 |
| PHOX2B | 31 | 32 | 61 | 10, 10, 15 |
| SIX6 | 35 | 36 | 59 | 15, 15, 20 |
| TITF1 | 39 | 40 | 60 | 20, 20, 30 |
| WT1 | 43 | 44 | 58 | 10, 10, 20 |
| BX161496 | 47 | 48 | 58 | 20, 20, 30 |
| CHR | 51 | 52 | 63 | 10, 10, 15 |
| GHSR | 55 | 56 | 60 | 20, 20, 30 |
| HOXB13 | 59 | 60 | 61 | 10, 10, 15 |
| HTR1B | 63 | 64 | 61 | 15, 15, 20 |
| NKX2-3 | 67 | 68 | 60 | 15, 15, 20 |
| ONECUT | 71 | 72 | 58 | 20, 20, 30 |
| POU4F | 75 | 76 | 58 | 5, 5, 10 |
| SLC38A4 | 79 | 80 | 64 | 10, 10, 15 |
| TMEM132D | 83 | 84 | 60 | 10, 10, 15 |

Thus, in one embodiment of the methods and uses of the invention, the oligonucleotide primer is e.g. SEQ ID NO: 15 and 16, and the annealing temperature is 66° C. Similarly, specific annealing temperatures for preferred oligonucleotide primers of the invention can be inferred from table 3.

### Elongation

The oligonucleotide primers annealed to the template is elongated to form an amplification product. The elongating temperature depends on optimum temperature for the polymerase, and is usually between 30 and 80 degrees Celsius. Typically, the elongating temperature is between 60 and 80 degrees Celsius, such as at least 60, at least 65, at least 68, at least 69, at least 70, preferably at least 71, at least 72, at least 73, at least 74, alternatively at least 75, at least 76, at least 77, at least 78, at least 79, at least 80 degrees Celsius. The PRC reaction mixture is incubated at the elongating temperature for 1 to 100 seconds, such as at least 1, at least 2, at least 3, at least 4, preferably at least 5, at least 6, at least 7, at least 8, at least 9, at least 10, alternatively at least 11, at least 13, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 seconds.

Elongation occurs in a buffered aqueous solution, preferably at a pH of 7-9.

The two oligonucleotide primers are added to the reaction mixture in a molar excess of primer: template especially when the template is genomic DNA which will ensure an improved efficiency. Deoxyribonucleoside triphosphates dATP, dCTP, dGTP, and dTTP are added to the reaction mixture, either separately or together with the primers. An appropriate agent for effecting the primer extension reaction, referred to and described elsewhere herein as an agent for polymerization is added to the reaction mixture. It is appreciated by a person skilled in the art that for PCR the agent for polymerisation preferable is a heat-stable polymerase enzyme, such as Taq polymerase.

### Cycling

The PCR method comprises incubating the nucleic acid at a cycle of different specific temperatures in order to control the steps of amplification. The amplification buffer and polymerase required for PCR are well known to people of skill within the art.

The PCR reaction mixture is incubated sequentially at the melting temperature, the annealing temperature and the elongating temperature, respectively, for a number of cycles. The PCR reaction may run between 10 and 70 cycles. Typically, the PCR reaction run between 25 and 55 cycles, such as at least 25, at least 30, at least 35, at least 40, preferably at least 45, at least 50 or at least 55 cycles.

In one embodiment, cycles of melting, annealing and elongation consist of 5-20, 5-20, and 2-30 seconds, respectively. Optimal cycling intervals are easily determined by those of skill in the art. Specific embodiments of cycle intervals for melting, annealing and elongation are indicated in table 3 together with preferred annealing temperature for the respective primers; i.e.:

Primers SEQ ID NO: 3 and 4: 15, 15, 20 seconds, respectively;
Primers SEQ ID NO: 7 and 8: 10, 10, 15 seconds, respectively;
Primer SEQ ID NO: 11 and 12: 20, 20, 30 seconds, respectively;
Primer SEQ ID NO: 15 and 16: 15, 15, 20 seconds, respectively;
. . . etc. . . . ; and
Primers SEQ ID NO: 83 and 84: 10, 10, 15 seconds, respectively;

PCR can be performed on a PCR machine, which is also known as a thermal cycler. Specifically, the thermal cycler may be coupled to a fluorometer, thus allowing the monitoring of the nucleic acid amplification in real time by use of intercalating fluorescent dyes, or other fluorescent probes. Applicable dyes according to the present invention include any DNA intercalating dye.

Suitable dyes include ethidium bromide, EvaGreen, LC Green, Syto9, SYBR Green, SensiMix HRM™ kit dye, however many dies are available for this same purpose.

Real-time PCR allows for easy performance of quantitative PCR (qPCR), which is usually aided by algorithms comprised in the software, which is usually supplied with the PCR machines.

The fluorometer can furthermore be equipped with software that will allow interpretation of the results. Such software for data analyses may also be supplied with the kit of the present invention.

Another variant of the PCR technique, multiplex PCR, enables the simultaneous amplification of many targets of interest in one reaction by using more than one pair of primers.

PCR according to the present invention comprise all known variants of the PCR technique known to people of skill within the art. Thus, the PCR technology comprise real-time PCR, qPCR, multiplex PCR.

### Oligonucleotide Primers

The oligonucleotide primer employed for amplification of modified nucleic acid is preferably a methylation-independent primer. The term “methylation-independent primer” refers to an oligonucleotide primer, which is capable of hybridizing to both methylated and unmethylated nucleic acid alleles and modified as well as unmodified alleles. A methylation-independent primer may not anneal with the exact same affinity to methylated/unmethylated nucleic acid alleles or modified/unmodified alleles.

The oligonucleotide primers of the present invention are capable of being employed in amplification reactions, wherein the primers are used in amplification of template DNA originating from either a methylation positive or amethylation negative strand. The preferred methylation-independent primers of the present invention comprise at least one CpG dinucleotide, as described below. Accordingly, in a methylation positive and bisulfite modified nucleic acid target sequence, the primer sequence will anneal to the nucleic acid template with a perfect match, wherein all of the nucleotides in a consecutive region of the primer forms base pairs with a complementary region in the nucleic acid target. However, in a methylation negative nucleic acid target after bisulfite modification, the methylation-independent primers of the present invention will anneal to the nucleic acid template with an imperfect match, wherein the primer sequence comprise a mis-match (i.e. the primer and template does not form base pairs) at the position of the unmethylated Cytosine at a CpG site in the nucleic acid template. Nonetheless, as the primers of the present invention are methylation-independent, the primers will hybridize to both methylation negative and methylation positive nucleic acid sequences after bisulfite modification, and the primers will form a perfect match with the target sequence of a methylated nucleic acid target and an imperfect match, where the primers and target nucleic acid sequence does not form base pairing at the positions of unmethylated Cytosine (which is converted by bisulfite to Uracil) at CpG sites.

The methylation-independent primers of the present invention will, due to the mis-match after bisulfite modification at positions of unmethylated cytosine of a CpG-site in the nucleic acid target sequence, hybridize less efficiently to a methylation negative nucleic acid sequence. However, by reducing the stringency of hybridization, the methylation-independent primers of the present invention are able to anneal to the nucleic acid target, also when the nucleic acid target comprise unmethylated CpG-sites, which have been modified by for example bisulfite treatment. In one example, the stringency is reduced by reducing the annealing temperature as described elsewhere herein.

The design of oligonucleotide primers suitable for nucleic acid amplification techniques, such as PCR, is known to people skilled within the art. The design of such primers involves analysis of the primer's melting temperatures and ability to form duplexes, hairpins or other secondary structures. Both the sequence and the length of the oligonucleotide primers are relevant in this context. The oligonucleotide primers according to the present invention comprise between 10 and 200 consecutive nucleotides, such as at least 60, at least 65, at least 70, at least 75, at least 80, at least 85, at least 90, at least 95, at least 100, at least 110, at least 120, at least 130, at least 140, at least 150, at least 160, at least 180 or at least 200 nucleotides. In a specific embodiment, the oligonucleotide primers comprise between 15 and 60 consecutive nucleotides, such as 15, 16, 17, 18, 19, 20, preferably 21, 22, 23, 24, 25, 26, 27, 28, 29, 30, such as 31, 32, 33, 34, 35, 36, 37, 38, 39, 40, alternatively at least 41, at least 42, at least 44, at least 46, at least 48, at least 50, at least 52, at least 54, at least 56, at least 58, or at least 60 consecutive nucleotides.

The methods employed for determining the methylation status of a nucleic acid according to the present invention, preferably comprise amplification of a modified nucleic acid by use of a methylation independent oligonucleotide primer. In one embodiment, the oligonucleotide primers of the present invention are able to hybridize to a nucleic acid sequence comprising CpG islands. In a preferred embodiment, at least one of the oligonucleotide primers according to the present invention comprises at least one CpG dinucleotide. In another embodiment of the present invention, the oligonucleotide primers comprise 2, alternatively 3, 4, 5, 6, 7, 8, 9 or 10 CpG dinucleotides. In even further embodiments, the oligonucleotide primers of the present invention comprise at least 10 CpG dinucleotides. In one preferred embodiment the at least one methylation-independent oligonucleotide primer comprises one CpG dinucleotide at the 5′-end of the primer.

The CpG dinucleotide may be located anywhere within the oligonucleotide primer sequence. However, in a preferred embodiment of the present invention, the at least one CpG dinucleotide is located in the 5′-end of the oligonucleotide primer. In another preferred embodiment, the at least one CpG dinucleotide constitute the first two nucleotides of the 5′-end. In an even further preferred embodiments of the present invention, the at least one CpG dinucleotide is located within the first 3, 4, 5, 6, 7, 8, 9, or 10 nucleotides of the 5′-terminus. In alternative embodiments, the at least one CpG dinucleotide is located within the first 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or 120 nucleotides of the 5′-terminus. In yet another embodiment, at least two CpG dinucleotides are located within the first 3, 4, 5, 6, 7, 8, 9, or 10 nucleotides of the 5′-terminus, or at least two CpG dinucleotides are located within the first 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or 120 nucleotides of the 5′-terminus.

The primers of the present invention may in one preferred embodiment comprise at least one CpG site, whereby annealing with a higher efficiency to a methylated than to an unmethylated template upon modification of unmethylated cytosine is achieved. The primers of the present invention comprise at least one CpG site. However, the primers comprise also for example two CpG sites.

The at least one CpG site is positioned in the 5′ end of the primer. For example within the first 10 nucleotides in the 5′ end of the primer, within the first 9 nucleotides in the 5′ end of the primer, within the first 8 nucleotides in the 5′ end of the primer, within the first 7 nucleotides in the 5′ end of the primer, within the first 6 nucleotides in the 5′ end of the primer, within the first 5 nucleotides in the 5′ end of the primer, within the first 4 nucleotides in the 5′ end of the primer or within the first 3 nucleotides in the 5′ end of the primer. In a preferred embodiment the CpG site is introduced immediately after the first nucleotide of the 5′ end of the primer.

Specific hybridization typically is accomplished by a primer having at least 10, for example at least 12, such as at least 14, for example at least 16, such as at least 18, for example at least 20, such as at least 22, for example at least 24, such as at least 26, for example at least 28, or such as at least 30 contiguous nucleotides, which are complementary to the target template. Often the primer will be close to 100% identical to the target template. However, the primer may also be 98% identical to the target template or for example at least 97%, such as at least 96%, for example at least 95%, such as at least 94%, for example at least 93%, such as at least 92%, for example at least 91%, such as at least 90%, for example at least 89%, such as at least 88%, for example at least 87%, such as at least 86%, for example at least 85%, such as at least 84%, for example at least 83%, such as at least 82%, for example at least 81%, such as at least 80%, for example at least 79%, such as at least 78%, for example at least 77%, such as at least 76%, for example at least 75%, such as at least 74%, for example at least 73%, such as at least 72%, for example at least 71%, such as at least 70%, for example at least 68%, such as at least 66%, for example at least 64%, such as at least 62% or for example at least 60% identical to the target template. If there is a sufficient region of complementary nucleotides, e.g., at least 10, such as at least 12, for example at least 15, such as at least 18, or for example at least 20, for example at least 30, such as at least 40, for example at least 50, such as at least 60, for example at least 70 nucleotides, then the primer may also contain additional nucleotide residues that do not interfere with hybridization but may be useful for other manipulations. Examples of such other residues may be sites for restriction endonuclease cleavage, for ligand binding or for factor binding or linkers.

The methylation-independent oligonucleotide primer of the present invention is designed to hybridize to nucleic acids in a sample. Importantly, the nucleic acids in that sample are treated with an agent, which modifies unmethylated cytosine in said nucleic acid. Thereby, any unmethylated Cytosine of CpG dinucleotides comprised in the nucleic acid are converted to Uracil as explained elsewhere herein. Consequently, in primers comprising a CpG dinucleotide, designed to hybridize with the complementary CpG dinucleotide of the nucleic acid of the sample, the CpG dinucleotide will only hybridize to the methylated CpG dinucleotide fraction of the nucleic acid. In the unmethylated fraction of CpG dinucleotides comprised in the nucleic acid of the sample, Cytosine are modified to uracil which does not hybridize with the CpG dinucleotide of the oligonucleotide primer.

The methylation-independent oligonucleotide primers according to the present invention are designed to comprise sufficient nucleotides for specific hybridization to the target nucleic acid sequence regardless of its original methylation status. In some embodiments the oligonucleotide primers also comprise one or more CpG dinucleotides, as specified elsewhere herein. These CpG dinucleotides only hybridize with the originally methylated alleles of the nucleic acids. Nevertheless, the oligonucleotide primers can still be functionally used for amplification of both originally methylated and unmethylated nucleic acids. The CpG dinucleotides are typically comprised in the 5′-terminus of the oligonucleotide primers, as described elsewhere herein. A primer-template mismatch within the 5′-terminus of the primer usually allow the primers to hybridize with the target nucleic acid, and still function as primers in an amplification reaction.

The presence of one or more mismatches between the primer and template affects the optimal annealing temperature of said oligonucleotide primer for use in amplification reactions. The more hybridizing nucleotides comprised on the oligonucleotide primers, the higher is the optimal annealing temperature. Consequently, amplification of methylated alleles of nucleic acids by CpG-containing oligonucleotide primers according to the present invention is favoured by increased annealing temperature. Conversely, amplification of unmethylated alleles is favoured by decreased annealing temperature. In the present invention, the PCR bias towards amplification of unmethylated alleles of a nucleic acid template is reversed by amplification of said nucleic acid template at a relatively higher annealing temperature, which favours oligonucleotide primer binding and priming of the methylated allele. By modulation of the primer annealing temperature, the priming of either the unmethylated modified allele or the methylated allele of the nucleic acid can be favoured. By increasing the annealing temperature below the theoretical optimum, the amplification of the methylated allele is favoured, while a decrease of the annealing temperature will tend to favour amplification of the unmethylated allele.

Besides annealing temperature, other factors also affect hybridisation to a target sequence of a methylation-independent primer. At highly stringent conditions, hybridization between perfect matching primer and target sequences are favoured, such as hybridization between a methylation-independent primer according to the present invention and a methylated target sequence upon cytosine modification. Less stringent conditions will tend to favour oligonucleotide primer binding, priming and amplification of the unmethylated allele. Modulation of temperature is one way of adjusting the stringency of hybridization, but the stringency of hybridization may also be modulated by adjusting buffer composition, and/or salt concentrations in the hybridization mixture, which is known to those of skill within the art. The present invention comprises any such method of modulating hybridization stringency to balance the PCR bias towards amplification of unmethylated template. However, modulation of temperature is preferred.

In one embodiment, the oligonucleotide primer of the present invention is selected from the group consisting of SEQ ID NO: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83 and 84. Methylation status is preferably determined for a gene mentioned in table 2 using the respective forward primer and reverse primer identified in table 2; i.e.

TITF1: forward primer=SEQ ID NO: 3 and reverse primer=SEQ ID NO: 4;
HOXB13: forward primer=SEQ ID NO: 7 and reverse primer=SEQ ID NO: 8;
. . . etc. . . . ; and
PHOX2B: forward primer=SEQ ID NO: 75 and reverse primer=SEQ ID NO: 76.

In one embodiment, an oligonucleotide primer of the present invention specifically hybridizes to regions within 1 kb of the gene loci of the present invention. In one embodiment, the oligonucleotide primers hybridize to a target nucleic acid sequence of a gene loci selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81), or the complement thereof.

In another embodiment of the present invention the oligonucleotide primer hybridizes to a target nucleic acid sequence of a gene loci selected from the group consisting of FLJ3247, GHSR, HOXB13, HTR1B, ONECUT, POU4F, WT1, LHX1, BX161496, CA10, NR2E1, PHOX2B, SIX6, SLC38A4, TITF, TMTM132D, CRH, NKX2-3 and HMX, or the complement thereof.

In another embodiment of the present invention the oligonucleotide primer hybridizes to a target nucleic acid sequence of a gene loci selected from the group consisting of PHOX2B, POU4F, SIX6, WT1, ONECUT, NKX2-3, FLJ32447 and/or CHR, or the complement thereof.

In another embodiment of the present invention the oligonucleotide primer hybridizes to a target nucleic acid sequence of a gene loci selected from the group consisting of TMEM132D, TITF1, NR2E1, CA10 and/or GHSR, or the complement thereof.

In another embodiment of the present invention the oligonucleotide primer hybridizes to a target nucleic acid sequence of a gene loci selected from the group consisting of FLJ3247, GHSR, ONECUT, POU4F, WT1, LHX1, BX161496, CA10, NR2E1, SIX6, SLC38A4, TITF, TMTM132D and HOXB13, or the complement thereof.

In another embodiment of the present invention the oligonucleotide primer hybridizes to a target nucleic acid sequence of a gene loci selected from the group consisting of HOXB13, FLJ3247, ONECUT, NR2E1 and TMTM132D, or the complement thereof.

In another embodiment of the present invention the oligonucleotide primer hybridizes to a target nucleic acid sequence of a gene loci selected from the group consisting of HOXB13, FLJ3247 and/or NR2E1, or the complement thereof.

In one specific embodiment, the oligonucleotide primer hybridizes to a target nucleic acid sequence of the PHOX2B gene locus, and/or the POU4F gene locus, and/or the SIX6 gene locus, and/or the WT1 gene locus, and/or the ONECUT gene locus, and/or the NKX2-3 gene locus, and/or the FLJ32447 gene locus, and/or the CHR gene locus.

In a preferred embodiment of the present invention the at least one oligonucleotide primer hybridizes to a target nucleic acid sequence selected from the group consisting of SEQ ID NO: 1, 5, 9, 13, 17, 21, 25, 29, 33, 37, 41, 45, 49, 53, 57, 61, 65, 69, 73, 77 and/or 81, or the complement thereof (non-modified strand); and/or the oligonucleotide prime hybridizes to a target nucleic acid sequence selected from the group consisting of SEQ ID NO: 2, 6, 10, 14, 18, 22, 26, 30, 34, 38, 42, 46, 50, 54, 58, 62, 66, 70, 74, 78 and/or 82, or the complement thereof (modified strand).

In another embodiment of the present invention the oligonucleotide primer hybridizes to a target nucleic acid sequence of HOXB13, FLJ3247, NR2E1, or the complement thereof.

In one embodiment, an oligonucleotide primer of the present invention specifically comprises or consists of 5-50, such as 5-30, such as 10-20 consecutive nucleotides of a subsequence of a gene loci selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81), or the complement thereof.

In particular, the present invention relates to oligonucleotide primer pairs, which span or comprise at least one CpG dinucleotide in a gene locus of the invention. The term “span” as used in this context is meant to indicated the at least one CpG site is located in the nucleic acid region between the primer pairs; i.e. the amplified nucleic acid region comprise at least one CpG dinucleotide. The term “comprising” as used in connection with “primers comprising at least one CpG dinucleotide is meant to specify that the oligonucleotide primer itself comprise a CpG site.

In a specific embodiment of the present invention the oligonucleotide primers comprises or consists of 5-50, such as 5-30, such as 10-20 consecutive nucleotides of a nucleic acid sequence of a gene loci selected from the group consisting of FLJ3247, GHSR, HOXB13, HTR1B, ONECUT, POU4F, WT1, LHX1, BC008699, BX161496, CA10, NR2E1, PHOX2B, SIX6, SLC38A4, TITF, TMTM132D, CRH, NKX2-3 and HMX, or the complement thereof.

In another embodiment of the present invention the oligonucleotide primer comprises or consists of 5-50, such as 5-30, such as 10-20 consecutive nucleotides of a nucleic acid sequence of a gene loci selected from the group consisting of FLJ3247, GHSR, ONECUT, POU4F, WT1, LHX1, BX161496, CA10, NR2E1, SIX6, SLC38A4, TITF, TMTM132D and HOXB13, or the complement thereof.

In another embodiment of the present invention the oligonucleotide primer comprises or consists of 5-50, such as 5-30, such as 10-20 consecutive nucleotides of a nucleic acid sequence of a gene loci selected from the group consisting of HOXB13, FLJ3247, ONECUT, NR2E1 and TMTM132D, or the complement thereof.

In another embodiment of the present invention the oligonucleotide primer comprises or consists of 5-50, such as 5-30, such as 10-20 consecutive nucleotides of a nucleic acid sequence of a gene loci selected from the group consisting of HOXB13, FLJ3247 and/or NR2E1, or the complement thereof.

In a preferred embodiment of the present invention the at least one oligonucleotide primer comprises or consists of 5-50, such as 5-30, such as 10-20 consecutive nucleotides of a nucleic acid sequence selected from the group consisting of SEQ ID NO: 1, 5, 9, 13, 17, 21, 25, 29, 33, 37, 41, 45, 49, 53, 57, 61, 65, 69, 73, 77 and/or 81, or the complement thereof (non-modified strand); and/or the oligonucleotide prime comprises or consists of 5-50, such as 5-30, such as 10-20 consecutive nucleotides of a nucleic acid sequence selected from the group consisting of SEQ ID NO: 2, 6, 10, 14, 18, 22, 26, 30, 34, 38, 42, 46, 50, 54, 58, 62, 66, 70, 74, 78 and/or 82, or the complement thereof (modified strand).

In another embodiment of the present invention the oligonucleotide primer comprises or consists of 5-50, such as 5-30, such as 10-20 consecutive nucleotides of a nucleic acid sequence of HOXB13, FLJ3247, NR2E1, or the complement thereof.

Thus, in the methods of the present invention for determining or prognosing breast cancer, determining a predisposition to breast cancer, categorizing or predicting breast cancer, or evaluating the risk of contracting a breast cancer, methylation status is preferably determined by amplifying at least one portion of a gene loci selected from FLJ3247, GHSR, HOXB13, HTR1B, ONECUT, POU4F, WT1, LHX1, BC008699, BX161496, CA10, NR2E1, PHOX2B, SIX6, SLC38A4, TITF, TMTM132D, CRH, NKX2-3 and HMX, using at least one primer pair selected from the nucleic acid sequences set forth in table 2 (SEQ ID NO: SEQ ID NO: 3/4, 7/8, 11/12, 15/16, 19/20, 23/24, 27/28, 31/32, 35/36, 39/40, 43/44, 47/48, 51/52, 55/56, 59/60, 63/64, 67/68, 71/72, and 75/76, respectively).

Detection of an amplification product can be performed by hybridizing the amplification product to an oligonucleotide probe, as described below. In a preferred embodiment, methylation status is determined by amplifying at least one portion of the respective at least one gene loci, and further employing at least one oligonucleotide probe which hybridizes to an amplification product selected from the group consisting SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or SEQ ID NO: 2, 6, 10, 14, 18, 22, 26, 30, 34, 38, 42, 46, 50, 54, 58, 62, 66, 70, 74, 78 and/or 82 and/or the complement thereof (modified strand) (Table 2).

In a preferred embodiment, the oligonucleotide probe comprise 10-100 consecutive nucleic acids selected from the group of sequences consisting SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or SEQ ID NO: 2, 6, 10, 14, 18, 22, 26, 30, 34, 38, 42, 46, 50, 54, 58, 62, 66, 70, 74, 78 and/or 82 and/or the complement thereof (modified strand).

One aspect of the invention also relates to the use of oligonucleotide primers of the present invention for determining or prognosing a breast cancer, determining a predisposition to breast cancer, categorizing or predicting breast cancer, or evaluating the risk of contracting a breast cancer. Thus, in one aspect, the present invention provides a use of oligonucleotide primers comprising a subsequence of a loci selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81) or the complement thereof for diagnosing breast cancer in a method of the invention as defined elsewhere herein.

In a preferred embodiment of the use of the invention, the primers are selected from the group set forth in table 2 (SEQ ID NO: SEQ ID NO: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83 and 84). In a preferred embodiment, the oligonucleotide primers comprising a sequence selected from the group consisting of SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand). In a preferred embodiment, the oligonucleotide primers comprising a subsequence selected from a gene loci selected from the group consisting of FLJ3247, HOXB13 and NR2E1, or the group consisting of FLJ3247 and NR2E1.

### Analysis of Amplified CpG-Containing Nucleic Acids

According to the present invention the nucleic acid (target) sample is subjected to an agent that converts an unmethylated cytosine to another nucleotide which will distinguish the unmethylated from the methylated cytosine. In a preferred embodiment the agent modifies unmethylated cytosine to uracil. The modifying agent can be sodium bisulphite. During the amplification process uracil will be converted to thymidine.

Thus, after conversion of unmethylated cytosines to uracils in the nucleic acid (target) sample, the subsequent PCR amplification converts uracils to thymine. As a consequence of the sodium bisulfite and PCR-mediated specific conversion of unmethylated cytosines to thymines, G:C base pairs are converted to A:T base pairs at positions, where the cytosine was methylated.

The difference in nucleic acid sequence at previously methylated (methylation positive) or unmethylated (methylation negative) cytosines allows for the analysis of methylation status in a sample. This analysis can comprise identifying cytosine residues, which have been converted to thymidine after amplification, as unmethylated cytosine residues, and identifying cytosine residues, which has not been converted under as methylated cytosine residues.

By this method, analysis of the amplified nucleic acid after treatment with a modifying agent such as sodium bisulphite and subsequent PCR amplification can reveal the methylation status of the target nucleic acid sequence. Thus, in one embodiment, the method for determining methylation status of a nucleic acid according to the present invention further comprises a step of analyzing the amplified nucleic acids.

Specifically, the subsequent analysis can be selected from the group consisting of melting curve analysis, high resolution melting analysis, nucleic acid sequencing, primer extension, denaturing gradient gel electrophoresis, southern blotting, restriction enzyme digestion, methylation-sensitive single-strand conformation analysis (MS-SSCA) and denaturing high performance liquid chromatography (DHPLC).

In one embodiment, the methylation status of the amplified containing nucleic acid is determined by any method selected from the group consisting of Methylation-Specific PCR (MSP), Whole genome bisulfite sequencing (BS-Seq), HELP assays, ChIP-on-chip assays, Restriction landmark genomic scanning, Methylated DNA immunoprecipitation (MeDIP), Pyrosequencing of bisulfite treated DNA, Molecular break light assays, and Methyl Sensitive Southern Blotting.

In another embodiment, the methylation status of the amplified containing nucleic acid is determined by a method selected from the group consisting methylation specific PCR, bisulfite sequencing, COBRA, melting curve analysis, or DNA methylation arrays.

In a preferred embodiment of the present invention, the analysis of the amplified nucleic acid region is melting curve analysis. In another preferred embodiment of the present invention, the analysis of the amplified nucleic acid is high resolution melting analysis (HRM).

### Melting Curve Analysis

Melting curve analysis or high resolution melting analysis exploits the fact that methylated and unmethylated alleles are predicted to differ in thermal stability because of the difference in GC contents after bisulphite treatment and PCR-amplification, which converts methylated C:G base pairs to A:T base pairs. This means that the melting curve profile of methylated (methylation positive) and unmethylated (methylation negative) alleles of PCR products originating from bisulfite modified methylated and unmethylated can be distinguished. Thus, the level of fluorescence changes, depending on the relative amount template; i.e. the relative amount of methylation positive and methylated negative alleles.

By comparing the melting curve profile of an unknown sample with different mixes of controls having known relative amounts of methylation positive and methylation negative alleles, the relative amount of methylation positive and methylation negative alleles of the unknown sample can be determined.

The melting curve profile of an amplification product according to the present invention is determined by the composition of methylated and unmethylated alleles in the nucleic acid sample. If the nucleic acid molecules of a sample are all methylation negative, all cytosines are converted to thymines, and the resulting PCR product will have a relatively low melting temperature compared to a methylated nucleic acid, which can be seen in its melting curve. If on the other hand, the nucleic acids comprised in the sample are methylation positive, the melting temperature of the PCR product will be relatively higher, and the melting curve is shifted, as fluorescence is observed at higher temperatures. If the nucleic acid sample comprises a mixture of methylated and unmethylated allelles, bisulphite treatment followed by amplification will result in two distinct amplification products. The unmethylated alleles will display a low melting temperature and the methylated alleles a high melting temperature, and the melting curve profile of such a sample shows fluorescence from both PCR products (methylated and unmetthylated). If only a subset of the CpG dinucleotides of the target sequence contain a methylated cytosine (heterogeneous methylation), the amplification product represents a pool of molecules, which are present in different cells of the tumor, with different melting temperatures, which leads to an overall intermediate melting temperature.

Melting curve analysis is performed by incubating the nucleic acid amplification product at a range of increasing temperatures. The temperature is increased from a starting temperature of at least 50 degrees Celsius, alternatively at least 55, at least 60, at least 62, at least 64, preferably at least 65, at least 66, at least 67, at least 68, at least 69, at least 70, at least 71, at least 72, at least 73, at least 74, at least 75, for example at least 76, at least 78, at least 80, at least 82, at least 84 degrees Celsius. The temperature is then increased to a final temperature of at least 70, at least 72, at least 74, at least 76, at least 78, at least 80, at least 82, at least 84, at least 86, preferably at least 88, at least 89, at least 90, at least 91, at least 92, at least 93, at least 94, at least 95, at least 96, at least 97, at least 98, at least 99, at least 100 degrees Celsius. In one embodiment, the temperature transitions from the starting temperature to the final temperature are a linear function of time. In a specific embodiment of the present invention, the linear transitions are at least 0.05 degrees Celsius per second, alternatively at least 0.01, at least 0.02, at least 0.03, at least 0.04, at least 0.06, at least 0.07, at least 0.08, at least 0.09, at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2, at least 3, at least 4, at least 5, at least 6, at least 7, at least 8, at least 9, at least 10 degrees Celsius per second. In a preferred embodiment, the melting curve analysis is performed by incubating the nucleic acid amplification product at increasing temperatures, from 70 to 95 degrees Celsius, wherein the temperature increases by 0.05 degrees per second.

The melting of the nucleic acid can be measured by a number of methods, which are known to people within skill of the art. One method involves use of agents, which fluoresce when bound to a nucleic acid in its double stranded conformation. Such agents include fluorescent probes or dyes, such as ethidium bromide, EvaGreen, LC Green, Syto9, SYBR Green, SensiMix HRM™ kit dye. Thus, in one embodiment, the melting curve analysis is performed by measurement of fluorescence. The melting of the nucleic acid amplification product according to the present invention can then be monitored as a decrease in the level of fluorescence from the sample. After measurement of the fluorescence the melting curves can be generated by plotting fluorescence as a function of temperature.

For direct comparison of melting curves from samples that have different starting fluorescence levels, the melting curves for data collected in HRM can be normalized, as described in the examples of the present invention. Such normalization methods are known to people of skill in the art. One preferred means of normalization include calculation of the ‘line of best fit’ in between two normalization regions before and after the major fluorescence decrease representing the melting of the amplification product. The ‘line of best fit’ is a statistical measure, designating a line plotted on a scatter plot of data (using a least-squares method) which is closest to most points of the plot. Calculation of the line of best fit is performed differentially on LightCycler and LightScanner, as illustrated in the examples of the present invention.

A platform with a combined thermal cycler and a fluorescence detector is ideal to perform intube melting analyses. Thus, in one embodiment, the melting curve analysis is performed on a thermal cycler coupled to a fluorometer, such as the Ligthcycler, LC480 (Roche) or the Rotorgene 6000 (Corbett Research). Thereby, the measurement of fluorescence, corresponding to the melting of the double stranded nucleic acid template, can be monitored in real time. In a specific embodiment, the melting curve analysis is performed immediately after amplification. This allows an in-tube methylation assay, wherein the amplification and melting curve analysis is performed sequentially without transferring the sample from the tube. This procedure reduces the risk of contamination of the sample as a result from handling during the methylation assay.

Melting curve analysis allows the determination of the relative amount of methylated nucleic acid in a sample. By comparison of the melting curve of an amplification product of nucleic acid for which methylation status in unknown sample with the melting curve of at least one standard sample comprising the corresponding amplification product for which methylation status is known, the relative amount of methylated CpG-containing nucleic acid can be estimated. Thus, the present invention relates to a method, wherein the relative amount of methylated nucleic acid is estimated by comparison the melting curve of at least one standard sample comprising said nucleic acid with a control level of methylation. In one embodiment of the present invention, said standard sample comprise any combination of methylated and unmethylated nucleic acid. In a specific embodiment, said standard sample comprise 100% methylated nucleic acid. In another specific embodiment, said standard sample comprise 100% unmethylated nucleic acid. In yet another specific embodiment, said standard sample comprise 50% methylated nucleic acid and 50% unmethylated nucleic acid. In even another specific embodiment, said standard sample comprise 0.1% 0.5%, 1%, 2%, 3%, 5%, 10%, 15%, 20%, 25%, 30%, 35%, 40%, 45%, 50%, 55%, 60%, 65%, 70%, 75%, 80%, 85%, 90%, 95%, or 100% methylated nucleic acid.

In one embodiment of the present invention, the relative amount of methylated nucleic acid in the nucleic acid sample is between 40-60%. In another embodiment, the relative amount of methylated nucleic acid in the nucleic acid sample is below 50%. In yet another embodiment, the relative amount of methylated nucleic acid in the nucleic acid sample is below 10%, below 1% or below 0.1%. Thus, the term “presence of methylation” and/or the term “methylation status” as used herein includes the relative amount of methylated nucleic acid in the nucleic acid sample of at least 0.1%, such as at least 1%, for example at least 10%, such as at least 20%, for example at least 30%, such as at least 50%, for example at least 70%, such as at least 90%, or for example at least 99%.

If the fluorescence level at an allele-specific peak melting temperature in the melting curve of an unknown sample is higher than that of a standard sample, then the relative amount of that specific allele (methylation positive or methylation negative) in the unknown sample is also higher than the relative amount of that allele in the standard sample. Thus, if a standard sample comprise 80% methylation positive alleles, for which the peak melting temperature is 70° C., and the fluorescence of an unknown sample at 70° C. is the same as that of the standard sample, then the relative level of methylation positive alleles in the unknown sample can be inferred to be around 80%. If the fluorescence of the unknown sample is less than the standard, the amount of methylation positive alleles can be inferred to be less than 80%, and if the fluorescence is higher than the standard, then the unknown sample comprise more than 80% methylation positive alleles. Thus, by comparing a melting curve fluorescence profile of an unknown sample with the profiles of standard samples with different compositions of methylation positive and methylation negative alleles, the level of methylated alleles of the unknown sample can be inferred.

The more standard samples, the more precise the relative amount of nucleic acids can be determined.

Thus, in one embodiment of the present invention, a higher fluorescence level at the peak melting temperature of the amplified nucleic acid sample than of a standard sample comprising a specific allele is indicative of a higher relative amount of that specific allele in that sample than in the standard sample. Conversely, a lower fluorescence level at the peak melting temperature of the amplified nucleic acid sample than of the standard sample comprising a specific allele is indicative of a lower relative amount of that specific allele in that sample than in the standard sample.

The “peak melting temperature” is mathematical a derivative of melting curve and refers to the temperature at which the largest discrete melting step occurs. The top of the peak corresponds to the major drop in fluorescence on the melting curve. The level of fluorescence at the peak melting temperature reflects the level of methylation for a given amplicon. Thus, two amplicons (derived from two different samples) may have peak melting temperatures of for example be 70° C., while having different fluorescence at this temperature, which then reflects that the amplicons have different methylation levels. The peak melting temperature corresponds to the highest level of the negative derivative of fluorescence (−dF/dT) over temperature versus temperature (T). A nucleic acid sample subjected to melting curve analysis may display more than one peak melting temperature. In a preferred embodiment of the present invention, the melting curve analysis display at least 1, 2 or 3 peak melting temperatures.

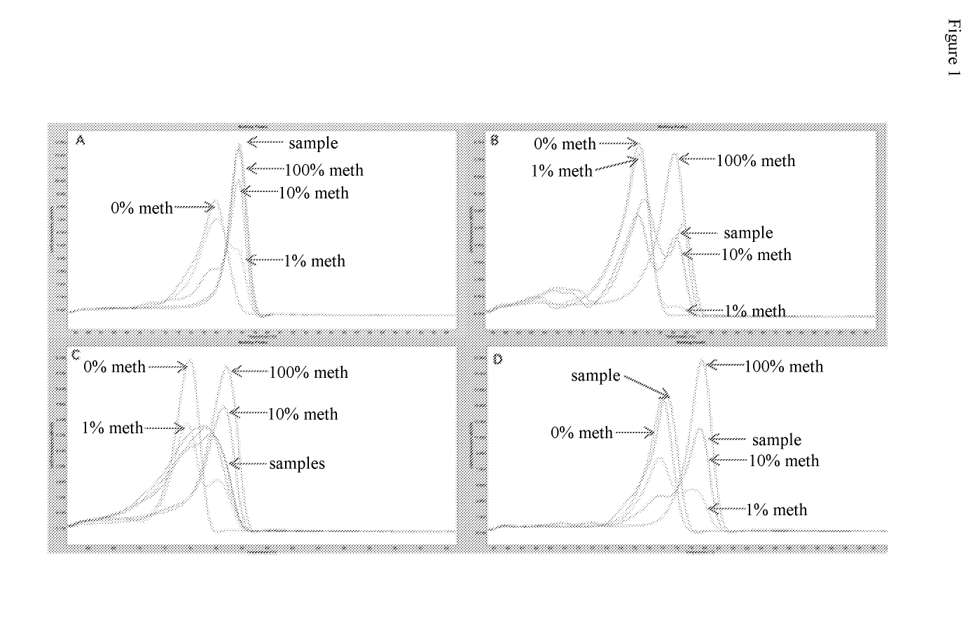

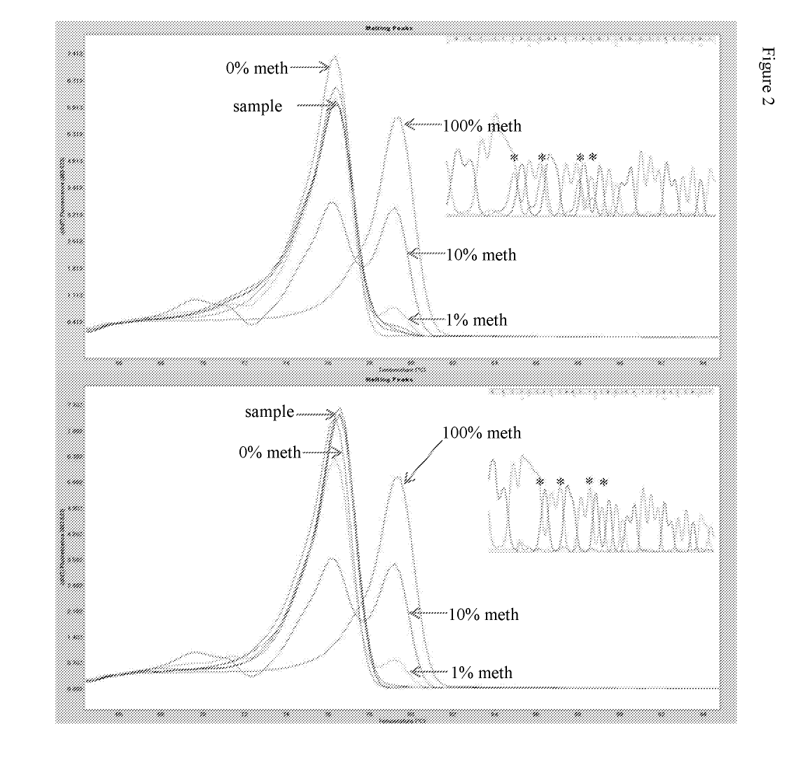

Melting curve analysis is illustrated in the examples herein below, and FIGS. 1-3.

### Nucleic Acid Sequencing

In another embodiment of the present invention, the method for analysis of the amplified nucleic acid is sequencing of the nucleic acid. By nucleic acid sequencing the order of nucleotides (base sequences) in the nucleic acid is determined. Sequencing is usually performed by extending a primer, which anneals to the nucleic acid sequence of interest. The primer is extended by a polymerase in the presence of deoxynucleonucleotides. Sequencing may also be performed by pyrosequencing.

In the dideoxy sequencing method 2,3-Dideoxyribose—a deoxyribose sugar lacking the 3 hydroxyl group is incorporated into the extended nucleic acid chain. When 2,3-Dideoxyribose is incorporated into a nucleic acid chain, it blocks further chain elongation. This method is also known as the Sanger method or chain termination method. The primer is extended in the presence of the normal dNTPs (A, T, G, C) and a small amount of 2,3-DideoxyriboseNTPs (ddNTP). The reactions are either performed in four separate reactions, one for each of the ddNTPs (ddATP, ddTTP, ddCTP, ddGTP), or in a joint reaction, wherein ddATP, ddTTP, ddCTP and ddGTP are coupled to different fluorescent dyes. The primers are then extended to variable lengths, each transcript being terminated upon incorporation of a ddNTP. The sequence of the nucleic acid of interest can then by read after denaturing polyacrylamid gel electrophoresis. Such sequencing techniques are known to people skilled within the art. Additionally, a number of different commercial kits are available for sequencing of nucleic acids.

### Primer Extension

In yet another embodiment of the present invention, the method for analysis of the amplified nucleic acid is primer extension. The primer extension method uses primers designed to hybridize with a target. The primers may end one base upstream of the position of the putative single nucleotide polymorphism, in this method, the C of a CpG dinucleotide. In the single nucleotide primer extension technique a single chain-ending nucleotide, such as a ddNTP, is added. The only one of the four nucleotides that will extend the primer is the one that is complementary. The identity of the added nucleotide, which reflects the methylation status, is determined in a variety of ways known to people of general skill within the art. For example, the chain-ending nucleotide may be radioactively labelled or coupled to a fluorescent dye, which can subsequently be identified.

### Restriction Enzyme Digestion

In a further embodiment, the method according to the present invention for analysis of the amplified nucleic acid is restriction enzyme digestion. Restriction enzymes can be divided into exonucelases and endonucleases. In a specific embodiment, the analysis of the amplified nucleic acid is restriction endonuclease digestion.

The method of the present invention results in the specific conversion of unmethylated cytosines to thymines, i.e. G:C base pairs are converted to A:T base pairs at positions, where a cytosine was methylated. This means that the nucleic acid sequence is changed, which may lead to disruption of a restriction endonuclease site or the change of a site specific for one restriction endonuclease to another restriction endonuclease.

In a preferred embodiment of the present invention, the modified and amplified nucleic acid is analyzed for disruption of a site specific for the endonuclease AciI, BstUI, HhaI, HinP1I, HpaII, HpyCH4IV, MspI, TaqaI, Fnu4HI, Hpy188I, HpyCH4III, NciI, ScrFI, BssKI, Hpy99I, Nt.CviPII. StyD4I, AatII, AccI, AcII, AfeI, AfIIII, AgeI, AvaI, BanI, BmgBI, BsaAI, BsaHI, BsaJI, BsaWI, BsiEI, BsiWI, BsoBI, BspDI, BspEI, BsrBI, BsrFI, BssHII, BssSI, BstBI, BtgI, Cac8I, ClaI, EaeI, EagI, FspI, HaeII, HincII, Hpy188III, KasI, MluI, MspA1I, NaeI, NarI, NgoMIV, NIaIV, NruI, PaeR7I, PmII, PvuI, SacII, SaII, SfoI, SmaI, SmII, SnaBI, TliI, TspMI, XhoI, XmaI, ZraI, RsrII, AscI, AsiSI, FseI, NotI, PspXI, SgrAI, AlwNI, DraIII, PflFI, Tth111I, AleI, BsaBI, MsII, PshAI, XmnI, AhdI, BglI, BsII, BstAPI, EcoNI, MwoI, PfIMI, BsmBI, FauI, BstXI, DrdI, SfiI, XcmI, HgaI, EciI, BceAI, BtgZI, MmeI, NmeAIII, BsaXI, BcgI, CspCl, BaeI, AccII, AspLEI, Bsh1236I, BsiSI, BstFNI, BstHHI, CfoI, HapII, Hin6I, HspAI or MaeII. The digested nucleic acid sample is subsequently analysed by for example gel electrophoresis.

### Denaturing Gradient Gel Electrophoresis

In another embodiment of the present invention, the method for analysis of the amplified nucleic acid is denaturing gradient gel electrophoresis (DGGE). In this technique, the modified and amplified nucleic acid is loaded on a denaturing gel. This techniques allows the resolution of nucleic acids with different melting temperatures, which is based on the conversion of C:G base pairs to A:T base pairs, explained elsewhere herein. For DGGE analysis the nucleic acid is subjected to denaturing polyacrylamide gel electrophoresis, wherein the gel contain an increasing gradient of denaturants, such as for example a combination of urea and formamide. The increasing denaturant concentration corresponds to increased temperature, and therefore, a gradient of denaturants mimics a temperature gradient within the gel. The concentrations of denaturants alone, however, are not sufficient to induce DNA melting. Therefore, the gel is immersed in an electrophoresis buffer kept at 54-60 degrees Celsius. When a nucleic acid molecule reaches a level of denaturant that matches the melting temperature of the lowest melting domain, a partially melted intermediate will be formed that moves very slowly. Small shifts in the melting temperature of the low melting domain induced by differences in G:C content will cause the domain to unwind at different concentrations of denaturant. Accordingly, the modified and amplified nucleic acid of the present invention will be retarded at different positions in the gel, providing the basis for physical separation between species with different G:C contents, which is reflective of methylation status.

### Southern Blotting

In another embodiment of the present invention, the method for analysis of the amplified nucleic acid is Southern blotting. In this procedure, the nucleic acid to be analysed are separated by gel electrophoresis and transferred to a nitrocellulose filter, whereto it is immobilized. After immobilization, the transferred nucleic acids can be identified by hybridization with specific probes comprising a complementary nucleic acid. After hybridization and removal of excess unbound probe, the amount of hybridized indicate whether the sequence of interest was represented in the nucleic acids immobilized on the nitrocellulose membrane. The probes are usually radioactively labelled for subsequent detection by radiography. The details of the southern blotting technique are well known to people of skill within the art.

### Methylation-Sensitive, Single-Strand Conformation Analysis (MS-SSCA)

MS-SSCA is a method of screening for methylation changes. MS-SSCA uses single-strand conformation analysis for the screening of an amplified region of bisulfite-modified nucleic acid. The amplified products are denatured and electrophoresed on a nondenaturing polyacrylamide gel, whereby the sequence differences between unmethylated and methylated sequences lead to the formation of different secondary structures (conformers) with different mobilities. Once the normal mobility pattern is established, any variation would indicate some degree of methylation.

### Denaturing High Performance Liquid Chromatography (DHPLC)

DHPLC is yet another technique for methylation screening of bisulfite-modified PCR products. As for other techniques mentioned herein, DHPLC identifies single nucleotide polymorphisms, which are arise after bisulfite treatment of unmethylated alleles of the CpG containing nucleic acid. The optimum temperature for DHPLC can be predicted by the sequence of the fully methylated product. Subsequently, the temperature is verified to obtain tight peaks. The retention time of the peak reflects methylation status, because the more unmethylated the target is, the less GC rich the PCR product is and the lower the retention time is.

### Kit

One aspect of the present invention relates to a kit for the detection of methylation status of a nucleic acid in a sample. A kit will typically comprise both a forward and a reverse primer to be used in the amplifying step of the present invention. The forward primer, the reverse primer or both may be a methylation-independent oligonucleotide primer as described herein. Thus, one aspect of the invention relates to a kit for determining breast cancer, predisposition to breast cancer, or categorizing or predicting the clinical outcome of a breast cancer, or monitoring the treatment of a breast cancer, and/or monitoring relapse of a previously treated breast cancer.

The kit of the invention comprise

i. an agent that (a) modifies methylated cytosine residues but not non-methylated cytosine residues; or (b) modifies non-methylated cytosine residues but not methylated cytosine residues; or (c) modifies a nucleic acid sequence in a methylation-dependent manner, and
ii. at least one pair of oligonucleotide primers that specifically hybridizes under amplification conditions to a region of a gene locus selected from the group consisting of FLJ3247, GHSR, HOXB13, HTR1B, ONECUT, POU4F, WT1, LHX1, BC008699, BX161496, CA10, NR2E1, PHOX2B, SIX6, SLC38A4, TITF, TMTM132D, CRH, NKX2-3 and HMX.

The agent is preferably a methylation-dependent endonuclease as described elsewhere herein, and/or an agent capable of modifying non-methylated cytosine residues but not methylated cytosine residues, such as a bisulphite compound as described elsewhere herein, for example sodium bisulphite.

Generally, the kit preferably comprises at least one oligonucleotide primer of probe of the present invention, as defined herein above. In a preferred embodiment, the kit comprise at least one oligonucleotide primer selected from the group consisting of SEQ ID NO: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83 and 84. In a more preferred embodiment, the kit comprises at least one primer pair selected from table 2; i.e. at least one primer pair identified as SEQ ID NO: SEQ ID NO: 3/4, 7/8, 11/12, 15/16, 19/20, 23/24, 27/28, 31/32, 35/36, 39/40, 43/44, 47/48, 51/52, 55/56, 59/60, 63/64, 67/68, 71/72, 75/76, 79/80 and 83/84.

The kit may also comprise one or more reference sample, in particular reference samples comprising a nucleic acid sequence selected from a gene locus selected from the group consisting of FLJ3247, GHSR, HOXB13, HTR1B, ONECUT, POU4F, WT1, LHX1, BC008699, BX161496, CA10, NR2E1, PHOX2B, SIX6, SLC38A4, TITF, TMTM132D, CRH, NKX2-3 and HMX. Thus, the kit may comprise a nucleic acid sequence selected from the group consisting of SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand).

For example, the at least one reference sample comprises 100% methylation positive reference nucleic acid, and/or 100% methylation negative reference nucleic acid. In a preferred embodiment the kit comprises at least two reference samples, wherein one of said reference samples comprises 100% methylation positive reference nucleic acid and a second reference sample comprises 100% methylation negative reference nucleic acid. The methylation positive and methylation negative reference nucleic acids may be mixed, by a person employing the kit, in ratios that are suitable for the detection of methylation in a particular sample. It is understood that reference samples in different ratios of methylation positive to methylation negative CpG-containing nucleic acids may be comprised in the kit. For example the kit may comprise at least one reference sample comprising 50% methylated and 50% non-methylated nucleic acid alleles of the respective genetic locus marker.

In particular, the nucleic acid comprised on the reference sample of the kit is preferably methylated (methylation positive) or non-methylated (methylation negative), and the kit preferably comprise two or more reference samples with different methylation status; i.e. different levels of methylation positive and methylation negative alleles. Thus, the specific nucleic acid alleles (e.g. alleles of the gene locus HTR1 B) of the reference sample may be unmethylated (0% methylated), 1%, 5%, 10%, 20%, 30%, 40%, 50%, 60%, 70%, 80%, 90%, 95%, or 100% methylated. The kit may thus comprise one reference sample with a nucleic acid sequence as defined above (e.g. selected from HOXB13 and/or SEQ ID NO: 5 or 6), which is unmethylated; and another reference sample with the same nucleic acid sequence (e.g. selected from HOXB13 and/or SEQ ID NO: 5 or 6), which is 100% methylated; and one or more samples comprising the same nucleic acid sequence (e.g. selected from HOXB13 and/or SEQ ID NO: 5 or 6) having different levels of intermediate methylation status, e.g. 10%, 50% and/or 90% methylation.

The kit preferably comprises the following combinations of one or more reference samples and primer pairs:

Reference sequence comprising gene locus TITF1: forward primer=SEQ ID NO: 3 and reverse primer=SEQ ID NO: 4;
HOXB13: forward primer=SEQ ID NO: 7 and reverse primer=SEQ ID NO: 8;
Reference sequence comprising gene locus NR2E1: forward primer=SEQ ID NO: 11 and reverse primer=SEQ ID NO: 12;
Reference sequence comprising gene locus HTR1B: forward primer=SEQ ID NO: 15 and reverse primer=SEQ ID NO: 16;
Reference sequence comprising gene locus HMX2: forward primer=SEQ ID NO: 19 and reverse primer=SEQ ID NO: 20;
Reference sequence comprising gene locus BC008699: forward primer=SEQ ID NO: 23 and reverse primer=SEQ ID NO: 24;
Reference sequence comprising gene locus SLC38A4: forward primer=SEQ ID NO: 27 and reverse primer=SEQ ID NO: 28;
FLJ32447: forward primer=SEQ ID NO: 31 and reverse primer=SEQ ID NO: 32;
Reference sequence comprising gene locus WT1: forward primer=SEQ ID NO: 35 and reverse primer=SEQ ID NO: 36;
Reference sequence comprising gene locus TMEM132D: forward primer=SEQ ID NO: 39 and reverse primer=SEQ ID NO: 40;
Reference sequence comprising gene locus NKX2-3: forward primer=SEQ ID NO: 43 and reverse primer=SEQ ID NO: 44;
Reference sequence comprising gene locus GHSR: forward primer=SEQ ID NO: 47 and reverse primer=SEQ ID NO: 48;
Reference sequence comprising gene locus ONECUT: forward primer=SEQ ID: 51 and reverse primer=SEQ ID NO: 52;
Reference sequence comprising gene locus LHX1: forward primer=SEQ ID NO: 55 and reverse primer=SEQ ID NO: 56;
Reference sequence comprising gene locus SIX6: forward primer=SEQ ID NO: 59 and reverse primer=SEQ ID NO: 60;
Reference sequence comprising gene locus CA10: forward primer=SEQ ID NO: 63 and reverse primer=SEQ ID NO: 64;
Reference sequence comprising gene locus CHR: forward primer=SEQ ID NO: 67 and reverse primer=SEQ ID NO: 68;
Reference sequence comprising gene locus POU4F: forward primer=SEQ ID NO: 71 and reverse primer=SEQ ID NO: 72;
Reference sequence comprising gene locus PHOX2B: forward primer=SEQ ID NO: 75 and reverse primer=SEQ ID NO: 76

The kit may also comprise at least one probe. Probes of the invention are defined herein above, and in a preferred embodiment, the kit comprise at least one oligonucleotide probe comprising 10-100 consecutive nucleic acids selected from the group of sequences consisting of SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group of sequences consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand). Thus, the kit of the invention preferably comprise at least one oligonucleotide probe which hybridizes to a nucleic acid sequence selected from the group consisting SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group of sequences consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand).

The kit of the invention preferably comprise at least one oligonucleotide probe which hybridizes to an amplification product generated by a primer pair selected from the group consisting of SEQ ID NO: SEQ ID NO: 3/4, 7/8, 11/12, 15/16, 19/20, 23/24, 27/28, 31/32, 35/36, 39/40, 43/44, 47/48, 51/52, 55/56, 59/60, 63/64, 67/68, 71/72, and 75/76.

The kit may also comprise additional reagents used in the amplifying step of the detection method as disclosed herein. Thus, the kit may further comprise deoxyribonucleoside triphosphates, DNA polymerase enzyme and/or nucleic acid amplification buffer. In another embodiment the kit further comprises an agent that modifies unmethylated cytosine nucleotides. Such an agent may for example be bisulfite, hydrogen sulfite, and/or disulfite reagent.

The kit may also comprise other components suitable for detection of methylation status. For example, the kit may comprise a methylation-sensitive restriction enzyme.

The kit may in preferred embodiments further comprise instructions for the performance of the detection method of the kit and for the interpretation of the results. The instructions for performing the method of the kit comprises for example information of particular annealing temperatures to be used for the at least one methylation-independent primers, as well as for example information on PCR cycling parameters. The kit may further comprise instructions for the interpretation of the results obtained by the method. For example how to interpret the amplified products subsequently analysed by melting curve analysis or methods as described elsewhere herein. Information of the interpretation of melting curve analysis is described elsewhere herein.

The kit may in preferred embodiments further comprise software comprising an algorithm for calculation of primer annealing temperature and interpretation of results. Preferred embodiments for the CpG-containing nucleic acid for which the methylation

It is appreciated that the kit may be used for evaluating a breast cancer in a human subject based on methylation status of specific genes as specified elsewhere herein.

### EXAMPLES

The invention relates to methylation biomarkers for breast cancer. Candidate marker genes were first identified by micro array analysis. Then, the ability of each of these methylation markers to distinguish between breast tumour tissue and healthy tissue was evaluated. From this analysis, 19 highly sensitive and specific methylation biomarkers were identified.

### Example 1

Background: Despite similar clinical and pathological features large number breast cancer patients experience different outcome of the disease. That together with the fact that the incidence of breast cancer is growing worldwide emphasizes an urgent need for identification of new biomarkers for early cancer detection and stratification of patients.

Methods: We have used ultra high-resolution microarrays to compare genome wide methylation patterns of breast carcinomas (N=20) and non-malignant breast tissue (N=5). A subset of the discovered differentially methylated regions (DMRs) was subsequently validated using Methylation Sensitive High Resolution Melting (MS-HRM) in a panel of breast carcinomas (N=275) and non-malignant controls (N=74).

Results: Based on microarray results we have selected 19 DMRs for large-scale screening of cases and controls using the MS-HRM technology. Analysis of MS-HRM results from the case cohort showed that all DMRs tested displayed drastic gains of methylation in the cancer tissue when compared to the levels in control tissue. Interestingly, we have observed two types of locus specific methylation, with loci undergoing either predominantly full or heterogeneous methylation during carcinogenesis. At the same time almost all tested DMRs (17 out of 19) displayed low-level methylation in non-malignant breast tissue independent of locus specific methylation pattern the in cases.

Conclusions: Specific loci seem to undergo heterogeneous or full methylation during carcinogenesis and loci hypermethylated in cancer frequently show low-level methylation in non-malignant tissue.

Impact: Screening for heterogeneous methylation and low level methylation at specific loci may be of high significance for the clinical applicability of methylation biomarkers.

### Introduction

The present example provides the basic steps of establishing methylation biomarkers for clinical use:
1. Discovery; where a genome wide screening in most of the cases is applied in search for the candidate biomarkers.
2. Initial clinical validation; where each biomarker is shown to be able to distinguish non-malignant (healthy tissue) form malignant (cancer tissue) with high specificity and sensitivity. At this point biomarkers that show 100% specificity can be considered for the early diagnostic applications.

The methylation biomarkers identified can then be subject to further analysis by retrospective validation; where archive material is used to determine if there is a significant correlation between specific methylation change(s) and the disease phenotype. A detailed record accompanying patient sample is critical for this part of the biomarker development process, and samples from various patient populations are highly advisable to be used in these studies. Once retrospective validation has been concluded, markers are further subjected to prospective validation, where the biomarker is used in clinical trials. The biomarker development process is preferably followed with long term monitoring of the impact of the biomarker's clinical use on different populations.

### Materials and Methods

### Clinical Sample Material

Twenty freshly frozen breast carcinomas were obtained from Aarhus University hospital and DNA from those samples was extracted using DNeasy Blood & Tissue Kit (Qiagen, Germany), according to the manufacturer protocol. Seventy-four non-malignant breast tissue samples from breast reduction surgeries were collected at the Department of Plastic Surgery, Aarhus University Hospital. The women undergoing breast reduction surgery were subjected to mammography and only women without signs of malignances were enrolled in this study. The breast tissue obtained from breast reductions can potentially differ from healthy breast tissue however due to ethical considerations this type of control material was the only available source for our experiments. DNA from breast reduction samples was also extracted using DNeasy Blood & Tissue Kit (Qiagen, Germany). Tumor DNA for screening analyses was obtained from 274 patients diagnosed with sporadic breast cancer, collected between 1992 and 1994 at Aarhus University Hospital. Complete information about the breast cancer cohort and DNA extraction produce was previously published; cf. Hansen L L, Andersen J, Overgaard J, Kruse T A. Molecular genetic analysis of easily accessible breast tumour DNA, purified from tissue left over from hormone receptor measurement. APMIS. 1998; 106:371-7; Hansen L L, Yilmaz M, Overgaard J, Andersen J, Kruse T A. Allelic loss of 16q23.2-24.2 is an independent marker of good prognosis in primary breast cancer. Cancer Res. 1998; 58:2166-9.

### Microarray Analyses

DNA for microarray experiments was extracted from 20 freshly frozen tumor tissue samples. After extraction the methylated DNA fragments were enriched in each sample using the MeDIP protocol (detailed description of the procedure can be found at www.nimblegen.com). The same procedure was applied for five tissue samples from breast reduction surgery serving as a control for the microarray experiments. Two fractions from each sample (MeDIP enriched and input) were labeled with Cy5 and Cy3, respectively, and co-hybridized to Human DNA Methylation 2.1M Deluxe Promoter Array (Roche/NimbleGen, Madison USA). Arrays were processed using the NimbleScan software to produce log 2 signal ratios at each probe. These ratios were averaged within each class of sample to produce a single set of mean ratios per class. The mean ratio sets were processed again by the NimbleScan software to generate a relative enrichment score at each probe for each class using a Kolmogorv-Smirnov test in a window around each probe. The enrichment scores for the mean ratios of each class were subtracted to produce a “differential score” indicating enrichment or depletion of signal in one group relative to the other, and a significance threshold was applied to the differential scores. Two or more consecutive significant differential scores within 500 bases of each other constituted a Differentially Methylated Region (DMR). Each DMR was mapped to the genome using the NimbleScan software (Roche/NimbleGen, Madison, USA). Lists of the annotated regions can be found in Supplementary data 1. For the results presented in this paper only the list of DMRs computed by subtraction of tumors from controls (hypermethylated DMRs) was manually mined for the candidate biomarkers. The potential candidate biomarkers (total of 24) with the highest differential score were selected for validation analyses. All validation experiments were performed using MS-HRM and verified by Sanger sequencing.

### Methylation Sensitive High Resolution Melting

All MS-HRM assays were designed according to previously published guidelines and optimized to allow for highly sensitive methylation detection; cf. Wojdacz T K, Dobrovic A, Hansen L L. Methylation-sensitive high-resolution melting. Nat Protoc. 2008; 3:1903-8; Wojdacz T K, Hansen L L, Dobrovic A. A new approach to primer design for the control of PCR bias in methylation studies. BMC Res Notes. 2008; 1:54. In each run a range of standards including: 0% (unmethylated, EpiTect Control DNA, Qiagen) 1%, 10% (of fully methylated template in unmethylated background and 100% (methylated, EpiTect Control DNA, Qiagen, Germany) was included to control for unbiased sensitivity of the detection. MS-HRM amplification was performed in triplicates. PCR mix consisted of 1× LightCycler®480 HRM Master Mix (Roche, Penzberg, Germany), 3 mM Mg⁺², 250-500 nM of each primer and 6 ng of template. PCR amplifications and HRM analyses were performed on the LighCycler® 480 platform (Roche, Penzberg, Germany). The conditions and primer sequences used in MS-HRM experiments are listed in Supplementary data 2. Bisulfite conversion of the clinical samples were performed with DNA Methylation-Gold™ Kit (ZymoResearch, Irvine, USA).

### Sequencing Analyses

To confirm the MS-HRM results, a subset of MS-HRM PCR products was sequenced using the Sanger method as previously described by Wojdacz T K, Moller T H, Thestrup B B, Kristensen L S, Hansen L L. Limitations and advantages of MS-HRM and bisulfite sequencing for single locus methylation studies. Expert Rev Mol Diagn. 10:575-80. In brief PCR products obtained from MS-HRM analyses were directly sequenced using the same primers as for the HRM analyses. To decrease the costs and labor of the sequencing the forward strand was sequenced from all the representative samples. In case of ambiguous results the reverse strand was additionally sequenced. Overall, we performed more than 300 sequencing reactions to confirm the MS-HRM results. Despite of the fact that we attempted to sequence very small PCR products (around 100 bp, for details see supplementary material 1), we have successfully and reproducibly confirmed the methylation status for each of HRM profile group described here (see results). We were not able to sequence WT1 and SIX6 PCR products (a very short PCR product) but high confidence of the sequencing data for all other assays allowed us to generalize the results for those two DMRs. The sequencing data for samples displaying low-level methylation for the HTR1B MS-HRM assay did not show methylation. However taking into account both superior sensitivity of the MS-HRM over sequencing, and the fact that all other low methylation profiles showed methylation on the sequencing data we have classified these samples as low-level methylated (Table 5).

### Statistical Analyses

The statistical analyses were performed with STATA 10.1 package and R statistics.

### Results

### Identification of Hypermethylated Loci

The data in this example provides identification of hypermethylated DMRs in breast cancer. A NimbleScan mapping of DMRs extracted from array data showed that DMRs detected in our sample panel could potentially be associated with 1000+ functional genomic elements. A direct indication of the accuracy of this part of the experimental approach was the finding that loci that previously have been reported to undergo hypermethylation during breast cancer pathogenesis e.g. PAX2, MTOD1 or PITX2 were also detected in our microarray experiments. For each called DMR our microarray data analysis workflow (see methods) provided us with a “differential score”. This value is in principle a derivative of the methylation difference between cases and controls, and the higher the score the more pronounced the methylation gain was observed when comparing both cases and controls. Therefore, 23 DMRs with highest “differential score” were selected for the MS-HRM based microarray validation experiments. MS-HRM assays were targeted to the called DMR or to the closest region that potentially could undergo differential methylation (e.g. CpG island—CGI). Results of the MS-HRM microarray validation corroborated the microarrays results for 21 of the selected target sequences. At this point of the experimental procedure, MS-HRM results indicated low-level methylation in the reference samples for a subset of the assays. However, the methylation level of those loci was significantly higher in cases. The use of quantitative properties of MS-HRM in this experiment was therefore critical. At the same time this finding indicates that the microarray used in the experiments is able to robustly detect relatively small relative differences in methylation between cases and controls. Two of the MS-HRM assays used in the microarray validation experiments did not confirm the microarray results. One possible explanation is that PCR based MS-HRM assays cover only approximately 100 bp, whereas the region with aberrant methylation called on the microarray can span large genomic regions and the methylation status within those sequence can differ. Design of PCR assays without prior knowledge of the methylation changes throughout the region is still challenging and can simply result in targeting the part of the regions that does not undergo cancer dependent methylation changes. The above explanation however does not rule out the possible technological limitations of the microarray technology, which can lead to false discoveries. Despite the fact that false positive results were present in our data set at a very low rate, the fact that they were present at all, underlines a critical importance of validation of the results obtained by any of the genome wide screening technology by PCR based methods.

### Initial Clinical Validation

All DMRs positively validated in the microarray validation experiments were subjected to clinical validation screening. The aim is to show the potential of the discovered DMR to distinguish between cancer (cases) and healthy controls. We have screened 275 cases of breast cancer with the same MS-HRM assays as used in the microarray validation process and 74 DNA samples from breast reductions surgeries representing healthy controls. The overall results of the initial clinical validation screening for the 19 DMRs are presented in Table 4 and 4.

TABLE 4
Frequencies of the DNA methylation in the control tissue samples.

| | | Low | Methylation | Methylation |
|---|---|---|---|---|
| | Samples | methylation** | negative | positive*** |
| Loci ID | no *: | (%) | (%) | (%) |
| TITF1 | 72 | 47 (65.3) | 25 (34.7) | 0 |
| HOXB13 | 72 | 13 (18.1) | 59 (81.9) | 0 |
| NR2E1 | 72 | 6 (8.3) | 66 (91.7) | 0 |
| HTR1B | 69 | 0 | 69 (100.0) | 0 |
| HMX2 | 69 | 45 **** (65.2) | 24 (34.8) | 0 |
| BC008699 | 72 | 21 (29.2) | 48 (66.7) | 3 (4.2) |
| SLC38A4 | 62 | 30 (48.4) | 29 (46.8) | 3 (4.8) |
| FLJ32447 | 70 | 48 (68.6) | 22 (31.4) | 0 |
| WT1 | 72 | 2 (2.8) | 70 (97.2) | 0 |
| TMEM132D | 70 | 37 (52.9) | 33 (47.1) | 0 |
| NKX2-3 | 68 | 68 (100.0) | 0 | 0 |
| GHSR | 72 | 2 (2.8) | 70 (97.2) | 0 |
| ONECUT | 71 | 24 (33.8) | 47 (66.2) | 0 |
| LHX1 | 72 | 0 | 54 (75.0) | 18 (25.0) |
| SIX6 | 69 | 8 (11.6) | 61 (88.4) | 0 |
| CA10 | 47 | 24 (51.1) | 23 (48.9) | 0 |
| CHR | 72 | 72 (100.0) | 0 | 0 |
| POU4F | 37 | 11 (29.7) | 26 (70.3) | 0 |
| PHOX2B | 72 | 72 (100.0) | 0 | 0 |

* Variable number of samples is reported due to clinical sample limitations
**Low methylation is referred to as methylation similar to the methylation level observed in the 1% methylation standard
***Methylation positive samples are referred to as samples displaying HRM profile characteristic for cases
**** Methylation level similar to the one observed in the 1-10% methylation standard

TABLE 5
Frequencies of DNA methylation in the cancer tissue samples.

| | | | | | | both |
|---|---|---|---|---|---|---|
| | | methylation | low | heterogeneous | Full | allele |
| Loci | samples | negative | methylation | methylation | methylation | present |
| ID | available * | (%) | (%) ** | (%) | (%) | (%) |
| TITF1 | 220 | 0 (0.0) | 38 (17.3) | 0 | 145 (65.9) | 37 (16.8) |
| HOXB13 | 239 | 18 (7.5) | 20 (8.4) | 0 | 23 (9.6) | 178 (74.5) |
| NR2E1 | 164 | 15 (9.1) | 14 (8.5) | 18 (11.0) | 89 (54.3) | 28 (17.1) |
| HTR1B | 162 | 28 (17.3) | 68 (42.0) | 20 (12.3) | 4 (2.5) | 42 (25.9) |
| HMX2 | 187 | 9 (4.8) | 1 (0.5) | 13 (7.0) | 164 (87.7) | 0 |
| BC008699 | 261 | 4 (1.5) | 4 (1.5) | 26 (10.0) | 206 (78.9) | 21 (8.0) |
| SLC38A4 | 234 | 0 | 16 (6.8) | 93 (39.7) | 80 (34.2) | 45 (19.2) |
| FLJ32447 | 264 | 0 | 28 (10.6) | 191 (72.3) | 48 (18.2) | 0 |
| WT1 | 218 | 44 (20.2) | 0 | 178 (81.7) | 21 (9.6) | 0 |
| TMEM132D | 218 | 6 (2.8) | 21 (9.6) | 154 (70.6) | 16 (7.3) | 21 (9.6) |
| NKX2-3 | 169 | 0 | 16 (9.5) | 108 (63.9) | 38 (22.5) | 7 (4.1) |
| GHSR | 260 | 56 (21.5) | 24 (9.2) | 180 (69.2) | 0 | 0 |
| ONECUT | 243 | 0 | 16 (9.5) | 134 (55.1) | 63 (25.9) | 30 (12.3) |
| LHX1 | 243 | 0 | 28 (11.5) | 145 (59.7) | 41 (16.9) | 29 (11.9) |
| SIX6 | 246 | 5 (2.0) | 11 (4.5) | 173 (70.3) | 55 (22.4) | 2 (0.8) |
| CA10 | 230 | 21 (9.1) | 48 (20.9) | 137 (59.6) | 24 (10.4) | 0 |
| CHR | 252 | 0 | 44 (17.5) | 192 (76.2) | 16 (6.3) | 0 |
| POU4F | 255 | 10 (3.9) | 6 (2.4) | 195 (76.5) | 44 (17.3) | 0 |
| PHOX2B | 256 | 0 | 3 (1.2) | 199 (77.7) | 53 (20.7) | 1 (0.4) |

* Variable number of the samples is reported due to clinical sample limitations
** Low methylation is referred to as methylation similar to the methylation observed in reference samples

The samples from the control group were subclassified based on the MS-HRM results into three groups (Table 4): samples displaying no methylation (1), samples showing low levels of methylation with low-level methylation defined as less methylation than 1% standard or any aberrations from the unmethylated profile in the range of the methylated epiallele melting temperature (2) and methylation positive samples (3). Interestingly, the cancer samples displayed significant variety of the HRM profiles. These profiles were subdivided into five groups (Table 5): methylation positive (1) and negative samples (2), samples displaying heterogeneous methylation pattern (3), samples showing only fully methylated melting profile (4), and the samples with both methylated and unmethylated alleles present (5). FIG. 1 illustrates examples of the different classes of HRM profiles used in our analyses. To confirm the accuracy of the classification of HRM profiles sequencing was performed of a subset of the samples from each of the HRM profile group and for each of the DMRs.

### Specificity of the Biomarkers and Low-Level Methylation in Controls

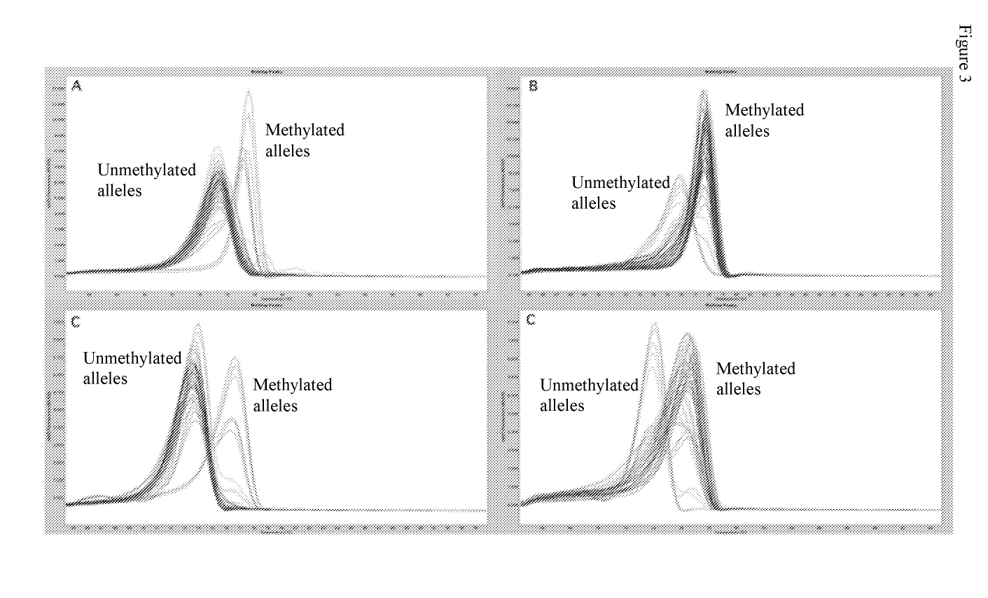

The specificity of each of DMR was evaluated based on the MS-HRM screening of the control tissue and the results are presented in Table 4. Interestingly we have observed low-level methylation in reference samples for 17 DMRs. The frequency of low-level methylation was as high as 100% for some of the loci (e.g. HOX2B). In addition three loci (BC008699, SLC38A4 and LHX1) showed low frequencies of the methylation levels similar to those observed in cancer tissue. These loci are suitable breast cancer biomarkers, as their specificity appears to be very low. FIG. 2 illustrates examples of the HRM scans with low methylation levels and verification of the results by sequencing. Overall, based upon the sequencing results we conclude that any aberration of the HRM profile from the unmethylated standard indicates the presence of methylation in the analyzed sample. Only one DMR in our panel (HMX2) showed methylation levels between 1 and 10% in the control tissue samples. The methylation levels in controls at all other DMRs were always below 1% when analyzing the data against the 1% methylation level standard. The high frequency of low levels of methylation in the control tissue hampers the specificity of the biomarkers. However the quantitative aspect of the MS-HRM technology allows establishing a cut off point for low-level methylation.

### High Methylation Levels in Cancer Samples

Despite that 17 of the DMRs from the identified panel showed low levels of methylation in the control tissue those levels seem to be insignificant when compared to the levels of the methylation observed at the same locus in the cancer samples. All tested DMRs, showed drastic gain of methylation during carcinogenesis. A general switch in methylation pattern from unmethylated in controls to methylated in cancer samples for two of the screened loci (SIX6 and BC008699) is illustrated in FIG. 3. Very few cancer samples in our cohort displayed low methylation levels similar to those observed in the control tissue (see Table 5 for details). For specificity calculations, methylation in those samples can be interpreted as a “normal” methylation level, when a cut-off point has been established based on analyses of methylation in the control group. The cut-off points for low levels of methylation can provide 100% specificity for the methylation biomarkers. However, before a cut-off point can be established the pathological significance of low levels of methylation within each DMR has to be evaluated.

### Two Types of Locus Specific Methylation

The frequencies of methylation in cancer samples are listed in Table 5. As shown twelve DMRs in our panel displayed predominant gain of heterogeneous methylation as result of breast cancer carcinogenesis affecting from 55% to 81% of the samples. Five of the DMRs showed very low frequencies of the heterogeneous methylation e.g TITF1 or HMX2 with 65% and 87% of the cancer samples showing presence of full methylation of both alleles. No heterogeneous methylation was seen for the HOXB13 assay but 74.5% of samples contained both methylated and unmethylated alleles at this DMR. Only one of the DMRs screened (SLC38A4) showed balanced frequencies of heterogeneous and full methylation of 39 and 34% respectively.

Interestingly, CGI targeted with our HOXB13 assay has previously been shown to undergo hypermethylation in colon cancer. Aberrant expression and methylation dependent expression of the HOXB13 gene has been shown in cancer. However, mutations within HOXB13 gene and the linkage analyses of the neighbouring region 17q21-22 found this region to be involved in development of different cancers. Our findings support those observations and the fact that our results seem to indicate deactivation of only one allele by DNA methylation, makes this phenomenon even more intriguing.

Overall, the present methylation screening results clearly show that the loci can undergo two types of methylation during carcinogenesis either heterogeneous or full methylation (at one or both alleles). The type of aberrant methylation seems to be locus specific and the mechanism of this process is unknown. This is the first study showing this phenomenon. At the same time the high locus specificity of the observed methylation changes shows that the results are not a technological artifact of the methodology used in this study but are biologically relevant.

### DISCUSSION

There is a strong research evidence to support the utility of methylation biomarkers in the entire process of clinical disease management, from screening for predisposition through detection of the condition to personalized treatment of the disease. The data presented here provides for the discovery and clinical validation of a number of breast cancer biomarkers. In the biomarker discovery step microarray technologies and NGS (next generation sequencing) are indispensable tools allowing in a single experiment to uncover a landscape of methylation changes throughout a cells genome. However, currently the complexity of those technologies does not allow for straightforward interpretation of the genome-wide screening results. The fact that we have observed false positive results in our microarray study illustrates the outmost importance of validation of the results of genome-wide screens with PCR based technologies to minimize over interpretation of the results. The validation step is especially important when complicated statistical modelling is used. In the study presented here we have used a simple statistical model for microarray data processing (see methods). Despite simplistic approach involving little data processing, validation experiments were necessary. This exemplifies the importance of PCR validation of the any gnome wide methylation based study before any conclusions are drawn. The results at the same time illustrates that simple statistical models can be very effective in discovery of disease dependent methylation aberrations.

The initial clinical validation of the biomarkers allows two questions to be answered. Firstly, the question of the recently emerging phenomenon of low-level methylation can be addressed in this step of biomarker development. The low-level methylation phenomenon has a significant influence on the specificity of the biomarker. Still, there is no consensus with regard to the origins of this phenomenon and its pathological significance. From the biomarker development perspective, methylation in healthy tissue should not be present for the biomarker to be highly informative. Our data show that low level methylation is very frequently present in healthy tissue. The MS-HRM technology allows setting a cut off point for the low levels of methylation, however before that can be done, the pathological insignificance of the low levels methylation has to be shown for each biomarker. This study demonstrates for the first time that due to its high prevalence, the evaluation of the low level methylation in healthy tissue is critical for biomarker development. The sequencing experiments performed by us provide evidence that low-level methylation that we observed in our controls is not technological artefact.

Heterogeneous methylation has been previously shown to be common for some loci but this phenomenon was not extensively researched due to the technological limitations of the technologies used in the field. The MS-HRM technology allowed us to perform methylation screening with the possibility to evaluate heterogeneous methylation in a large number of samples. Our experiments show for the first time a trend for loci to undergo two types of methylation during carcinogenesis with some loci undergoing full methylation and others heterogeneous methylation. Moreover the heterogeneous methylation is as specific to the locus as full methylation. Full methylation of the locus normally abolishes the transcription. Heterogeneous methylation may not be sufficient to abolish transcription of the gene, but may only interfere with the transcription process or be a “passenger” of carcinogenesis process. With the current technological advances, the discovery and development of new biomarkers may seem an uncomplicated task. However, despite substantial research evidence for the utility of methylation biomarkers in clinical disease management methylation biomarkers are still fares from routine diagnostic use. The results presented here show that development of methylation biomarkers for clinical use is complicated from a technological point of view, and that potential methylation biomarkers for cancer identified by microarray technology must be clinically validated before the methylation biomarkers can be used in routine clinical practice.

### Example 2

### MeDIP

MeDIP was performed on the 25 samples following a specific protocol from NimbleGen, Roche.
1. DNA extraction was performed with as described in DNA extraction.
2. DNA fragmentation was performed using Mse I restriction enzyme (5′-T↓TTA) (New England Biolabs, R0525S)
a. 6 μg of DNA from each sample was digested with 24 U of Mse I (10,000 U/ml) overnight at 37° C. in a solution containing 10×NEB4 buffer (provided with the Mse I), BSA (1 μg/μl) (Invitrogen, 15561-020), and water. The reactions was stopped by heating the samples for 20 min at 65° C.
b. Samples were purified using QIAquick PCR Purification Kit (Qiagen, 28104) as described in “Purification”.
c. DNA concentration was measured using a NanoDrop (Thermo Scientific) and fragmentation was verified on a 2% agarose gel, using 300 ng of Mse I digested
DNA. Fragments were in the range of 200-1,000 bp to obtain efficient immunoprecipitation.
3. Immunoprecipitation of methylated DNA
Monoclonal mouse anti 5-methyl cytidine antibodies, 100 μg/100 μl (Eurogentec, I-MECY-0100) was used in a 1:1 ratio to DNA.
a. 1.25 μg of Mse I digested DNA was diluted to a final volume of 300 μl in TE buffer (TE buffer: 10 mM TrisHCl, pH7.5, and 1 mM EDTA).
b. The samples were denatured at 95° C. for 10 min and immediately cooled on ice for 5 min to obtain single stranded DNA, necessary for antibody binding. Samples were kept at 4° C.
c. Control (input) DNA: 250 ng DNA, equivalent to 60 μl, was removed from each sample and stored at −20° C.
d. Immunoprecipitated (IP) DNA: 60 μl of 5×IP buffer was added to the remaining 240 μl DNA solution (5×IP buffer: 50 ml 100 mM Na-phosphate (pH 7.0), 14 ml 5M NaCl, 2.5 ml 10% triton X-100 (Sigma-Adrich, 93426), and 33.5 ml water)
e. 1.3 μg antibody was added to each sample and the DNA-antibody mixture was incubated overnight at a rotating platform at 4° C.
4. Binding of DNA:Antibody mixture to beads.
a. Protein A agarose beads (Invitrogen 15918-014) was washed twice using PBS-BSA 0.1% (10×PBS: Invitrogen, 70013-032)
i) The beads were re-suspended by shaking. 48 μl of beads was added to 1.5 ml microcentrifuge tube and centrifuged at 6,000 rpm for 2 min at 4° C. Supernatant was removed.
ii) 600 μl of PBS-BSA 0.1% was added to each sample and samples incubated on a rotating platform for 5 min at 4° C. Subsequently the samples were centrifuged at 6,000 rpm for 2 min at 4° C. The supernatant was removed and this step was repeated.
b. Beads were re-suspended in 24 μl 1×IP buffer (1×IP buffer: 5× diluted 5×IP buffer) and added to the DNA:Antibody mixture and incubated on a rotating platform for 2 hours at 4° C.
c. DNA:Antibody:Beads mixture was washed three times using 1×IP buffer to remove unbound unmethylated DNA from the solution. For each wash, 1 ml of 1×IP was added to the mixture and incubated on a rotating platform for 5 min at 4° C. and centrifuged at 6,000 rpm for 5 min at 4° C. followed by removal of supernatant.
5. Degradation of beads and antibodies
a. Each mixture was re-suspended in 250 μl digestion buffer (5 ml 1M TrisHCl (pH 8.0), 2 ml 0.5M EDTA, 5 ml 10% SDS (Sigma-Aldrich, L-4522), and 88 ml water).
b. 7 μl of Proteinase K mix (10 mg/mi) (Roche Applied Science, 03115836001) was added to the mixture to digest the beads and antibodies. The mixtures incubated overnight at a rotating platform at 55° C.
6. Purification of methylated DNA
a. 250 μl phenol (Sigma-Aldrich, P-4557) was added to each sample. Samples were vortexed for 30 seconds and centrifuged at 14,000 rpm for 5 min at room temperature. Supernatant was transferred to a new 1.5 ml microcentrifuge tube.
b. 250 μl Chloroform:isoamyl alcohol (24:1) (Sigma-Aldrich, C0549) was added to each sample and proceeded as above.
c. 1 μl glycogen (20 mg/ml) (Roche Applied Science, 10901393001) was added, followed by the addition of 20 μl 5 M NaCl and 500 μl absolute ethanol (Sigma-Aldrich, E702-3).
d. The DNA was precipitated at −80° C. for 30 min followed by centrifugation at 14,000 rpm for 15 min at 4° C. The supernatant was removed and discarded.
e. The pellet was washed with 500 μl 70% ice-cold ethanol (diluted absolute ethanol, (Sigma-Aldrich, E702-3) and centrifuged at 14,000 rpm for 5 min at 4° C. The supernatant was removed and the pellet was completely dried in a SpeedVac.
f. The samples were resuspended in 30 μl 10 mM TrisHCl (pH 8.5) and the DNA concentration was measured using a NanoDrop. The expected DNA yield in each sample was 10-15 ng/μl.
7. Amplification of immunopecipitated (IP) and control (Input) DNA using Whole Genome Amplification Kit 2 (WGA2, Sigma-Aldrich, WGA2-50RXN)) as described in “3. WGA 2 amplification” to get higher DNA yield.
8. After each round of amplification, samples were purified using QIAquick PCR Purification Kit, Qiagen, see section “4. Purification”, and DNA concentration was measured using a NanoDrop.

### WGA 2 Amplification

10 ng of IP and Input DNA were used for amplification. A positive control DNA sample, Control Human Genomic DNA, is provided in the WGA2 kit (Sigma-Aldrich, WGA2-50RXN) and is also amplified using the same procedure.

### 1. Fragmentation

a. 1 μl of 10× fragmentation buffer was added to each 10 μl DNA (1 ng/μl) sample (IP, input, and positive control DNA sample) in a 200 μl PCR tube. The Control Human Genomic DNA (5 ng/μl) was diluted to yield 1 ng//μl.
b. The solution was heated at 95° C. for 4 min in a thermal cycler and subsequently cooled on ice.

### 2. Library Preparation

a. 2 μl of 1× Library Preparation Buffer and 1 μl Library Stabilization Solution was added to each sample. Samples were vortexed thoroughly, centrifuged briefly, heated in a thermal cycler at 95° C. for 2 min, and cooled on ice.
b. 1 μl of Library Preparation Enzyme was added to each sample. Samples were vortexed thoroughly, centrifuged briefly, and run in a thermal cycler using the following program (table 6)

TABLE 6
Incubation program for WGA2 libary preparation.

| Temperature | Time |
|---|---|
| 16 | 20 |
| 24 | 20 |
| 37 | 20 |
| 75 | 5 |
| 4 | Hold |

### 3. Amplification

A master mix containing 7.5 μl 10× Amplification Master Mix, 47.5 μl Nuclease-Free water, and 5 μl WGA DNA Polymerase was added to each sample. Samples were vortexed thoroughly, centrifuged briefly, and run in a thermal cycler using the following program (table 7).

TABLE 7
Amplification program for WGA2

| Step | Temp. | Time |
|---|---|---|
| Denaturation | 95 | 3 |
| 14 cycles as follows | | |
| Denature | 94 | 15 |
| Anneal/Extend | 65 | 5 |

Amplification of DNA was verified on a 2% agarose gel and the DNA amount was measured using a NanoDrop.

### WGA3 Re-Amplification

Re-amplification was performed using WGA3 (Sigma-Aldrich WGA3-50RXN) 1. 10 μl of 1 ng/μl WGA2 amplified, and purified, DNA was added to a 200 μl PCR tube.

2. A amplification mix containing 7.5 μl 10× Amplification Master Mix, 47.5 μl Nuclease-Free water, 5 μl WGA DNA Polymerase, and 3 μl 10 mM dNTP mix was added to each sample. Samples were vortexed thoroughly, centrifuged briefly, and run in a thermal cycler using the same program as for WGA2 amplification (table 2). Amplification of DNA sequences was verified on a 2% agarose gel and the DNA amount was measured using a NanoDrop.

### Purification

DNA samples were purified using the QIAquick PCR Purification Kit (Qiagen, 28104). All centrifugations were performed at 13,000 rpm at room temperature.

1. 5 volumes of Buffer PB were added to 1 volume of sample and each solution was transferred to a QIAquick spin column with a collection tube. Samples were centrifuged for 1 min. Flow-through was discarded.
2. 0.75 μl of Buffer PE was added to each sample. Samples were centrifuged for 1 min and flow-through was discarded. Samples were centrifuged for 1 min again to remove ethanol in Buffer PE.
3. The spin column was transferred to a new 1.5 microcentrifuge tube and 50 μl of Buffer EB was placed on the QIAquick membrane. Samples were centrifuged for 1 min. This step was repeated to get a higher DNA yield.
4. All samples were stored at −20° C.

### Sequences

The names of the assays refer to closes functional element to the microarray call as mapped by NimbleScan software (e.g. mRNA or gene locus)

```
Primer binding sites - in red
Original sequence was translated to bisulfite modified sequence using:
http://www.urogene.org/methprimer/index1.html
Fragment BC008699: chr14: 37, 123, 572-37, 123, 689 (UCSC Genome Browser
on Human Feb. 2009 (GRCh37/hg19) Assembly), Length: 118 bp
SEQ ID NO: 1
  121 CCTGCCCAGTTCCCGGGAGGGCCAACCCCAGCCAGTAAGAAGACCTGAGGCGTAGGATCC
          ::||:::||||::++||||||::||::::||::||||||||||::|||||++||||||::
  121 TTTGTTTAGTTTTCGGGAGGGTTAATTTTAGTTAGTAAGAAGATTTGAGGCGTAGGATTT
SEQ ID NO: 2
  181 CTGCGCAGGAGGTTGCAGGAATGCCCCCGCTAGCCGCAAGGTTCCTGCTGGCCTGTAGAG
          :||++:|||||||||:|||||||::::++:|||:++:||||||::||:|||::|||||||
  181 TTGCGTAGGAGGTTGTAGGAATGTTTTCGTTAGTCGTAAGGTTTTTGTTGGTTTGTAGAG
  241 CTTTCGTGATCCCCGCCAAGATGCGAACAGTAAGGTCCTCGTATGGATCGCAGTTTTTGT
          :|||++||||:::++::||||||++||:||||||||::|++|||||||++:|||||||||
  241 TTTTCGTGATTTTCGTTAAGATGCGAATAGTAAGGTTTTCGTATGGATCGTAGTTTTTGT
(SEQ ID NO: 3)
F: AGGATTTTTGCGTAGGAGGTTGT
(SEQ ID NO: 4)
R: ACGATCCATACGAAAACCTTACTA
Conditions:
BC008699 f4-r3, MSHRM MM, Mg3mM, 65 deg., 15, 15, 20 sec. pr. cycle
FragmentCA10: chr17: 50, 235, 319-50, 235, 417 (UCSC Genome Browser on
Human Feb. 2009 (GRCh37/hg19) Assembly), Length 99 bp
SEQ ID NO: 5
  181 GCCGCTGGTGCGAAGAGAAGAGACACGCGAGCGGGGAGACCTCCAAGGCAGCGAGGCATC
          +:++:|||||++|||||||||||:|++++||++||||||::|::||||:||++|||:||+
  181 GTCGTTGGTGCGAAGAGAAGAGATACGCGAGCGGGGAGATTTTTAAGGTAGCGAGGTATC
SEQ ID NO: 6
  241 GGACATGTGTCAGCACATCTGGGGCGCACATCCGTCGAGCCCGAGGGGAGATTTGCCGGA
          +||:||||||:||:|:||:|||||++:|:||:++|++||::++||||||||||||:++||
  241 GGATATGTGTTAGTATATTTGGGGCGTATATTCGTCGAGTTCGAGGGGAGATTTGTCGGA
  301 ACAATTCAAACTGCGATATTGATCTTGGGGGTGACTGTCCCTGGCCGGCTGTCGGGTGGG
          |:||||:|||:||++|||||||:|||||||||||:|||:::|||:++|:|||++||||||
  301 ATAATTTAAATTGCGATATTGATTTTGGGGGTGATTGTTTTTGGTCGGTTGTCGGGTGGG
(SEQ ID NO: 7)
F: GAGCGGGGAGATTTTTAAGGT
(SEQ ID NO: 8)
R: AAATTATTCCGACAAATCTCCCCT
Conditions:
CA10 f3-r3, MSHRM MM, Mg3mM, 63 deg., 10, 10, 15 sec. pr. cycle
Fragment FLJ3247: chr2: 223, 162, 979-223, 163, 068 (UCSC Genome Browser
on Human Feb. 2009 (GRCh37/hg19) Assembly), Length 90 bp
SEQ ID NO: 9
      1 CGCTCAGAAGCCGGTTCACCTCCTTCTCCACCGCGGCATTTCCAAAACAACAGGGACAAG
          ++:|:|||||:++|||:|::|::||:|::|:++++|:||||::||||:||:|||||:|||
      1 CGTTTAGAAGTCGGTTTATTTTTTTTTTTATCGCGGTATTTTTAAAATAATAGGGATAAG
SEQ ID NO: 10
    61 TCTCCCCGGCTCGCCGCAGGCCTGACCGCCCAGCTCCGCCAGGATTTGCAGAGAGCAGCG
          |:|:::++|:|++:++:|||::|||:++:::||:|:++::||||||||:||||||:||++
    61 TTTTTTCGGTTCGTCGTAGGTTTGATCGTTTAGTTTCGTTAGGATTTGTAGAGAGTAGCG
  121 CGCTCCATTTGCAGAAAGGAAATCGAGTAGGTCCTCGCCCCCGACTGGTGCTTCTTGGGG
          ++:|::|||||:|||||||||||++|||||||::|++::::++|:|||||:||:||||||
  121 CGTTTTATTTGTAGAAAGGAAATCGAGTAGGTTTTCGTTTTCGATTGGTGTTTTTTGGGG
(SEQ ID NO: 11)
F: GCGGTATTTTTAAAATAATAGGGATAAG
(SEQ ID NO: 12)
R: CGCGCTACTCTCTACAAATCCTAA
Conditions:
FLJ3247 f1-r1, MSHRM MM, Mg3mM, 61 deg., 20, 20, 30 sec. pr. cycle
Fragment HMX2: chr10: 124, 902, 806-124, 902, 920 (UCSC Genome Browser
on Human Feb. 2009 (GRCh37/hg19) Assembly), Length 115 bp
SEQ ID NO: 13
  241 AGAACAACTAGGCGGGATGTACTTTTGAGCCCTGCCGGGTGTCTCCGATCGGAGTCTGGG
          ||||:||:||||++|||||||:|||||||:::||:++|||||:|:++||++||||:||||
  241 AGAATAATTAGGCGGGATGTATTTTTGAGTTTTGTCGGGTGTTTTCGATCGGAGTTTGGG
SEQ ID NO: 14
  301 GTTGAGATTTGGGCTGCACTTGTCCCCGGTGTGTCTCTCCGGCGGAGTACCCTGAAGGTG
          |||||||||||||:||:|:||||:::++||||||:|:|:++|++|||||:::||||||||
  301 GTTGAGATTTGGGTTGTATTTGTTTTCGGTGTGTTTTTTCGGCGGAGTATTTTGAAGGTG
  361 CACGAGGTGGGGAGCATAGGCTGAGGTGGGTAATCGGGTCCTGGATAGAAACACAACCCT
          :|++||||||||||:|||||:|||||||||||||++|||::||||||||||:|:||:::|
  361 TACGAGGTGGGGAGTATAGGTTGAGGTGGGTAATCGGGTTTTGGATAGAAATATAATTTT
(SEQ ID NO: 15)
F: GCGGGATGTATTTTTGAGTTTTGT
(SEQ ID NO: 16)
R: CTCGTACACCTTCAAAATACTCC
Conditions:
HMX2 f3-r3, MSHRM MM, Mg3mM, 66 deg., 15, 15, 20 sec. pr. cycle
Fragment HS3ST2: chr16: 22, 824, 824-22, 824, 930 (UCSC Genome Browser
on Human Feb. 2009 (GRCh37/hg19) Assembly), Length 107 bp
SEQ ID NO: 17
  421 GCGAGCCGCCGGGGTGTGAGTCAGCGCGCTGGGGGCTAAGAAGCTGGGTGAATAGTCACG
          |++||:++:++||||||||||:||++++:||||||:|||||||:||||||||||||:|++
  421 GCGAGTCGTCGGGGTGTGAGTTAGCGCGTTGGGGGTTAAGAAGTTGGGTGAATAGTTACG
SEQ ID NO: 18
  481 GAATCTCACTCACGCTCGGCTCCTCCACCCATCCCGTCTACAGCGCGTGTCCCAGTCCAG
          ||||:|:|:|:|++:|++|:|::|::|:::||::++|:||:||++++|||:::|||::||
  481 GAATTTTATTTACGTTCGGTTTTTTTATTTATTTCGTTTATAGCGCGTGTTTTAGTTTAG
  541 GGCGTGCGTGCGCTCGGTGTCCGATTCCGGGCTGTGTGTGTCCATTTGGCGAGATGTCGA
  541 GGCGTGCGTGCGTTCGGTGTTCGATTTCGGGTTGTGTGTGTTTATTTGGCGAGATGTCGA
(SEQ ID NO: 19)
F: GCGCGTTGGGGGTTAAGAAGT
(SEQ ID NO: 20)
R: CACGCACGCCCTAAACTAAAACA
Conditions:
HS3ST2 f1-r1, MSHRM MM, Mg3mM, 63 deg., 10, 10, 15 sec. pr. cycle
Fragment LHX1: chr17: 35, 297, 992-35, 298, 091 (UCSC Genome Browser on
Human Feb. 2009 (GRCh37/hg19) Assembly), Length 100 bp
SEQ ID NO: 21
  961 AGCGCCAACGTGTCGGACAAGGAAGCGGGTAGCAACGAGAATGACGACCAGAACCTGGGC
          ||++::||++|||++||:|||||||++|||||:||++|||||||++|::||||::||||+
  961 AGCGTTAACGTGTCGGATAAGGAAGCGGGTAGTAACGAGAATGACGATTAGAATTTGGGC
SEQ ID NO: 22
1021 GCCAAGCGGCGGGGACCGCGCACCACCATCAAAGCCAAGCAGCTGGAGACGCTGAAGGCC
          +::|||++|++||||:++++:|::|::||:||||::|||:||:||||||++:||||||:+
1021 GTTAAGCGGCGGGGATCGCGTATTATTATTAAAGTTAAGTAGTTGGAGACGTTGAAGGTC
(SEQ ID NO: 23)
F: GTCGGATAAGGAAGCGGGTAGT
(SEQ ID NO: 24)
R: CGTCTCCAACTACTTAACTTTAATAATAATA
Conditions:
LHX1 f1-r1, MSHRM MM, Mg3mM, 63 deg., 10, 10, 15 sec. pr. cycle
Fragment NR2E1: chr6: 108, 485, 970-108, 486, 088 (UCSC Genome Browser
on Human Feb. 2009 (GRCh37/hg19) Assembly), Length 119 bp
SEQ ID NO: 25
  241 GGCGCCCCACTAAGGAGGACACAGGCTCTGGTGTGTGTGGTGTGCGAGACCCCGAGCTCG
          +|++::::|:|||||||||:|:|||:|:||||||||||||||||++|||:::++|+|++
  241 GGCGTTTTATTAAGGAGGATATAGGTTTTGGTGTGTGTGGTGTGCGAGATTTCGAGTTCG
SEQ ID NO: 26
  301 AGGCCGAGCCAAGGCTGGGCAGAAAGTTGCAATCACGTGCTGTCGGAGCCCACTGGAGCG
          |||:++||::||||:||||:|||||||||:|||:|++||:|||++|||:::|:|||||++
  301 AGGTCGAGTTAAGGTTGGGTAGAAAGTTGTAATTACGTGTTGTCGGAGTTTATTGGAGCG
  361 CACAGCCCGCTCCCCCTGGGACGCCCAGGCGGAGGACCTGCTGCGCCCTCCCAGGGCTCG
          :|:||::++:|:::::|||||++:::|||++|||||::||:||++:::|:::||||:|++
  361 TATAGTTCGTTTTTTTTGGGACGTTTAGGCGGAGGATTTGTTGCGTTTTTTTAGGGTTCG
(SEQ ID NO: 27)
F: CGAGGTCGAGTTAAGGTTGGGT
(SEQ ID NO: 28)
R: ACCCTAAAAAAACGCAACAAATCCTC
Conditions:
NR2E1 f1-r1, MSHRM MM, Mg3mM, 65 deg., 10, 10, 15 sec. pr. cycle
Fragment PHOX2B: chr4: 41, 753, 256-41, 753, 361 (UCSC Genome Browser on
Human Feb. 2009 (GRCh37/hg19) Assembly), Length 106 bp
SEQ ID NO: 29
  601 CCTTACTGCACCTGGGGTGTGTCTCCGCGTGGTGCAGAGCGCGCGCTCTACTCCGGAAGC
          ::|||:||:|::||||||||||:|:++++|||||:||||++++++:|:||:|:++||||:
  601 TTTTATTGTATTTGGGGTGTGTTTTCGCGTGGTGTAGAGCGCGCGTTTTATTTCGGAAGT
SEQ ID NO: 30
  661 TACGGCCGGGTGCCGCGCCACCGCTGTGCGCCCTGGGCCTGATCCCTACGCCCTAGTCGA
          ||++|:++||||:++++::|:++:||||++:::||||::||||:::||++:::||||++|
  661 TACGGTCGGGTGTCGCGTTATCGTTGTGCGTTTTGGGTTTGATTTTTACGTTTTAGTCGA
  721 GTGCAGGGCAGGGCAATTTCGCCGTGGGTCCT
          |||:||||:||||:|||||++:++|||||::|
  721 GTGTAGGGTAGGGTAATTTCGTCGTGGGTTTT
(SEQ ID NO: 31)
F: GGGTGTGTTTTCGCGTGGTGT
(SEQ ID NO: 32)
R: TCGACTAAAACGTAAAAATCAAACCCAAAA
Conditions:
PHOX2B f1-r1, MSHRM MM, Mg3mM, 61 deg., 10, 10, 15 sec. pr. cycle
Fragement SOX6-SIX: chr14: 60, 973, 980-60, 974, 117
on Human Feb. 2009 (GRCh37/hg19) Assembly), Length 138 bp
SEQ ID NO: 33
      1 CCTGGCCAGAAGCTCCGGGATCGCAGCCCTCCCGGGTCCGGCTTCATCCCTGCCCGGCCA
          ::|||::|||||:|:++||||++:||:::|::++|||:++|:||:||:::||::++|::|
      1 TTTGGTTAGAAGTTTCGGGATCGTAGTTTTTTCGGGTTCGGTTTTATTTTTGTTCGGTTA
SEQ ID NO: 34
    61 CCGAGGCCCTCTTTTTCTGCACCGCGGATTCTCCTCCGCCTGCGTGTTCGGGGCCCTTGT
          :++|||:::|:|||||:||:|:++++||||:|::|:++::||++||||++|||:::||||
    61 TCGAGGTTTTTTTTTTTTGTATCGCGGATTTTTTTTCGTTTGCGTGTTCGGGGTTTTTGT
  121 ATCCGATGTTTCTTTCTAAAAGTTGTCCTTCCGGCTGATTCGGAAGTCGCTCCAAGGGAA
          ||:++||||||:|||:||||||||||::||:++|:|||||++|||||++:|::|||||||
  121 ATTCGATGTTTTTTTTTAAAAGTTGTTTTTTGGTTGATTCGGGAAGTCGTTTTAAGGGAA
(SEQ ID NO: 35)
F: GTTTTTTCGGGTTCGGTTTTATTTTTGT
(SEQ ID NO: 36)
R: CCGAATCAACCGAAAAAACAACTTTTAA
Conditions:
SOX6f1-SIXr1, MSHRM MM, Mg3mM, 59 deg., 15, 15, 20 sec. pr. cycle
Fragment TITF: chr14: 36, 992, 328-36, 992, 413 (UCSC Genome Browser on
Human Feb. 2009 (GRCh37/hg19) Assembly), Length 86 bp
SEQ ID NO: 37
  661 CGCTGGCCCCTCGCGGAGCTTTCCCTGGCGCGACCTCACACGGTCGCTGCCTCTATTCCG
          ++:|||::::|++++|||:|||:::|||++++|::|:|:|++||++:||::|||:::|||
  661 CGTTGGTTTTTCGCGGAGTTTTTTTTGGCGCGATTTTATACGGTCGTTGTTTTTATTTCG
SEQ ID NO: 38
  721 ACCACGCTCTGCTTCGCTGGCTGCGGCTCCGCCAGGAATCCGAGGGGGCGCAGGCCCAGG
          |::|++:|:||:||++||||:||++|:|:++::||||||:++||||||++:|||:::|||
  721 ATTACGTTTTGTTTCGTTGGTTGCGGTTTCGTTAGGAATTCGAGGGGGCGTAGGTTTAGG
(SEQ ID NO: 39)
F: GGAGTTTTTTTTGGCGCGATTTTATA
(SEQ ID NO: 40)
R: AATTCCTAACGAAACCGCAACCAA
Conditions:
TITF f2-r1, MSHRM MM, Mg3mM, 60 deg., 20, 20, 30 sec. pr. cycle
Fragment WT1: chr11: 32, 456, 867-32, 456, 962 (UCSC Genome Browser on
Human Feb. 2009 (GRCh37/hg19) Assembly), Length 96 bp
SEQ ID NO: 41
1981 CCGGCTCCGGGACACACGTGGAAGCCGGGTCCTGCAGCAAGAGGAAGTCCAGGATCGCGG
          :++|:|:++|||:|:|++||||||:++|||::||:||:||||||||||::|||||++++|
1981 TCGGTTTCGGGATATACGTGGAAGTCGGGTTTTGTAGTAAGAGGAAGTTTAGGATCGCGG
SEQ ID NO: 42
2041 CGAGGAGACGGCGGGGCCCGGGCGCCTGGGCTGCCGTCCCGGCTCTGGGTGGGTGGGTGG
          ++||||||++|++|||::++||++::||||:||:++|::++|:|:|||||||||||||||
2041 CGAGGAGACGGCGGGGTTCGGGCGTTTGGGTTGTCGTTTCGGTTTTGGGTGGGTGGGTGG
(SEQ ID NO: 43)
F: TATACGTGGAAGTCGGGTTTTGTA
(SEQ ID NO: 44)
R: CCAAAACCGAAACGACAACCCAAA
Conditions:
WT1 f3-r2, MSHRM MM, Mg3mM, 58 deg., 10, 10, 20 sec. pr. cycle
Fragment BX161496: chr14: 36, 992, 359-36, 992, 485 (UCSC Genome Browser
on Human Feb. 2009 (GRCh37/hg19) Assembly), Length 127 bp
SEQ ID NO: 45
  661 CGCTGGCCCCTCGCGGAGCTTTCCCTGGCGCGACCTCACACGGTCGCTGCCTCTATTCCG
          ++:|||::::|++++|||:|||:::|||++++|::|:|:|++||++:||::|:||||:++
  661 CGTTGGTTTTTCGCGGAGTTTTTTTTGGCGCGATTTTATACGGTCGTTGTTTTTATTTCG
SEQ ID NO: 46
  721 ACCACGCTCTGCTTCGCTGGCTGCGGCTCCGCCAGGAATCCGAGGGGGCGCAGGCCCAGG
          |::|++:|:||:||++:|||:||++|:|:++::||||||:++||||||++:|||:::|||
  721 ATTACGTTTTGTTTCGTTGGTTGCGGTTTCGTTAGGAATTCGAGGGGGCGTAGGTTTAGG
  781 CTCGGCCCTAGATGCGCGGAATCGCCATCAGCCTTTGCTTACACCAGCGTGGCCGCAGGG
          :|++|:::||||||++++||||++::||:||::||||:|||:|::||++|||:++:||||
  781 TTCGGTTTTAGATGCGCGGAATCGTTATTAGTTTTTGTTTATATTAGCGTGGTCGTAGGG
(SEQ ID NO: 47)
F: GTTGTTTTTATTTCGATTACGTTTTGTTT
(SEQ ID NO: 48)
R: CCACGCTAATATAAACAAAAACTAATAA
Conditions:
BX161496 f1-r1, MSHRM MM, Mg3mM, 58 deg., 20, 20, 30 sec. pr. cycle
Fragment CHR; chr8: 67, 090, 430-67, 090, 484 (UCSC Genome Browser on
Human Feb. 2009 (GRCh37/hg19) Assembly), Length 55 bp
SEQ ID NO: 49
      1 GCATACACACGTACACAGGCAGGGGCAGCCGGCTCCGCGGCGCACATCGCGGCAGCTCAG
          |:|||:|:|++||:|:|||:|||||:||:++|:|:++++|++:|:||++++|:||:|:||
      1 GTATATATACGTATATAGGTAGGGGTAGTCGGTTTCGCGGCGTATATCGCGGTAGTTTAG
SEQ ID NO: 50
    61 GCAACGCAAAGTTGGTGGCGTGTTCCGTCCAGGCGCTCCCTACCTTCCCAGGCGCTTCGC
          |:||++:|||||||||||++||||:++|::|||++:|:::||::||:::|||++:||++:
    61 GTAACGTAAAGTTGGTGGCGTGTTTCGTTTAGGCGTTTTTTATTTTTTTAGGCGTTTCGT
(SEQ ID NO: 51)
F: TATACGTATATAGGTAGGGGTAGT
(SEQ ID NO: 52)
R: ACGAAACACGCCACCAACTTTA
Conditions:
CHR f1-r1, MSHRM MM, Mg3mM, 63 deg., 10, 10, 15 sec. pr. cycle
Fragment GSHP: chr3: 172, 167, 580-172, 167, 683 (UCSC Genome Browser on
Human Feb. 2009 (GRCh37/hg19) Assembly), Length 104 bp
SEQ ID NO: 53
      1 GGACGCGGTCTGTGCGTCTCCTGCTCAGAGCCAGAAATCAGCACCCGAAGGCATGAGACT
          |||++++||:||||++|:|::||:|:||||::||||||:||:|::++||||:||||||:|
      1 GGACGCGGTTTGTGCGTTTTTTGTTTAGAGTTAGAAATTAGTATTCGAAGGTATGAGATT
SEQ ID NO: 54
    61 GCCAGTTGCCAGCGAATTCACAAATCCGACCGGCCCCTCCCGGCCCACCGACCTCGGGAC
          |::|||||::||++||||:|:||||:++|:++|::::|::++|:::|:++|::|++|||:
    61 GTTAGTTGTTAGCGAATTTATAAATTCGATCGGTTTTTTTCGGTTTATCGATTTCGGGAT
  121 CGCCCCAGGAACATATTCAGCACTGTGGCCAGCGCCACATCCATCCTACCGCAAAGCGCC
          ++::::|||||:|||||:||:|:|||||::||++::|:||::||::||:++:||||++:+
  121 CGTTTTAGGAATATATTTAGTATTGTGGTTAGCGTTATATTTATTTTATCGTAAAGCGTC
(SEQ ID NO: 55)
F: GATTGTTAGTTGTTAGCGAATTTATAAATT
(SEQ ID NO: 56)
R: ATATAACGCTAACCACAATACTAAATATA
Conditions:
GHSR f1-r1, MSHRM MM, Mg3mM, 60 deg., 20, 20, 30 sec. pr. cycle
Fragment HOX B13: chr17: 46, 810, 857-46, 810, 932 (UCSC Genome Browser on
Human Feb. 2009 (GRCh37/hg19) Assembly), Length 76 bp
SEQ ID NO: 57
  241 GCAGCGCGACGCTCCCCTCTCCCGAAAGGTTGGCTCCACGGTCCCGCCGGCCGCGCAGGT
          |:||++++|++:|::::|:|::++|||||||||:|::|++||::++:++|:++++:||||
  241 GTAGCGCGACGTTTTTTTTTTTCGAAAGGTTGGTTTTACGGTTTCGTCGGTCGCGTAGGT
SEQ ID NO: 58
  301 CTGGCTGAACTGCTTGGGGTCGCCCGGCTCCTCTCG
          :|||:||||:||:|||||||++::++|:|::|:|++
  301 TTGGTTGAATTGTTTGGGGTCGTTCGGTTTTTTTCG
(SEQ ID NO: 59)
F: GACGTTTTTTTTTTTCGAAAGGTTGGTTTT
(SEQ ID NO: 60)
R: ACGACCCCAAACAATTCAACCAAAC
Conditions:
HOX B13 f1-r1, MSHRM MM, Mg3mM, 61 deg., 10, 10, 15 sec. pr. cycle
Fragment HTR1B: chr6: 78, 173, 811-78, 173, 908 (UCSC Genome Browser on
Human Feb. 2009 (GRCh37/hg19) Assembly), Length 98 bp
SEQ ID NO: 61
1561 GACGGAGCCATAAAAGGGGGGACACGGGGGCTGGAGTTGCGGCTGCTCGGGCCGCGCCGC
          ||++|||::|||||||||||||:|++||||:||||||||++|:||:|++||:++++:++:
1561 GACGGAGTTATAAAAGGGGGGATACGGGGGTTGGAGTTGCGGTTGTTCGGGTCGCGTCGT
SEQ ID NO: 62
1621 CGCCACCGCCACCCTGGTCCCACGGGAGCCACTCGGAGCCATGCCACTGGGTGCGCGGGT
          ++::|:++::|:::||||:::|++||||::|:|++|||::|||::|:||||||++++|||
1621 CGTTATCGTTATTTTGGTTTTACGGGAGTTATTCGGAGTTATGTTATTGGGTGCGCGGGT
(SEQ ID NO: 63)
F: GGATACGGGGGTTGGAGTTG
(SEQ ID NO: 64)
R: CGCGCACCCAATAACATAACT
Conditions:
HTR1B f2-r2, MSHRM MM, Mg 3mM, 61 deg., 15, 15, 20 sec. pr. cycle
Fragment NKX2-3: chr10: 101, 293, 836-101, 293, 948 (UCSC Genome Browser
on Human Feb. 2009 (GRCh37/hg19) Assembly), Length 113 bp
SEQ ID NO: 65
  481 GTCCTTGAACCCGTGGCACTCGGTAGAGAGAGAGGAGATGATCGGAAAGTGCGTGGGAAC
          +|::|||||::++|||:|:|++||||||||||||||||||||++|||||||++||||||:
  481 GTTTTTGAATTCGTGGTATTCGGTAGAGAGAGAGGAGATGATCGGAAAGTGCGTGGGAAT
SEQ ID NO: 66
  541 AATGTCTATTCCGCGCGACCATAGCTCTCACATCCCTAAGGCGCCAGCCTTTTTTGAAAA
          |||||:||||:++++++|::||||:|:|:|:||:::|||||++::||::|||||||||||
  541 AATGTTTATTTCGCGCGATTATAGTTTTTATATTTTTAAGGCGTTAGTTTTTTTTGAAAA
  601 TCCGTAACGTTTTGCTTTGTGTCCCAGGCTGCGGGCCTAATAGAAAACGCGCCGAACTTG
          |:++|||++|||||:|||||||:::|||:||++||::||||||||||++++|++||:|||
  601 TTCGTAACGTTTTGTTTTGTGTTTTAGGTTGCGGGTTTAATAGAAAACGCGTCGAATTTG
(SEQ ID NO: 67)
F: AAAGTGCGTGGGAATAATGTTTATTT
(SEQ ID NO: 68)
R: AAACCCGCAACCTAAAACACAAAA
Conditions:
NKX2-3 f2-r2, MSHRM MM, Mg3mM, 60 deg., 15, 15, 20 sec. pr. cycle
Fragment ONECUT: chr18: 55, 103, 594-55, 103, 702 (UCSC Genome Browser
on Human Feb. 2009 (GRCh37/hg19) Assembly), Length 109 bp
SEQ ID NO: 69
  181 CATGAACAACCTCTACAGTCCCTACAAGGAGATGCCCGGCATGAGCCAGAGCCTGTCCCC
          :|||||:||::|:||:|||:::||:|||||||||::++|:|||||::||||::|||:::+
  181 TATGAATAATTTTTATAGTTTTTATAAGGAGATGTTCGGTATGAGTTAGAGTTTGTTTTC
SEQ ID NO: 70
  241 GCTGGCCGCCACGCCGCTGGGCAACGGGCTAGGCGGCCTCCACAACGCGCAGCAGAGTCT
          +:|||:++::|++:++:||||:||++||:||||++|::|::|:||++++:||:|||||:|
  241 GTTGGTCGTTACGTCGTTGGGTAACGGGTTAGGCGGTTTTTATAACGCGTAGTAGAGTTT
  301 GCCCAACTACGGTCCGCCGGGCCACGACAAAATGCTCAGCCCCAACTTCGACGCGCACCA
          |:::||:||++||:++:++||::|++|:||||||:|:||::::||:||++|++++:|::|
  301 GTTTAATTACGGTTCGTCGGGTTACGATAAAATGTTTAGTTTTAATTTCGACGCGTATTA
(SEQ ID NO: 71)
F: GAGATGTTCGGTATGAGTTAGAGTT
(SEQ ID NO: 72)
R: ACGAACCGTAATTAAACAAACTCTACTA
Conditions:
ONECUT f1-r1, MSHRM MM, Mg 3mM, 58 deg., 20, 20, 30 sec. pr. Cycle
Fragment POU4f: chr4: 147, 561, 490-147, 561, 548 (UCSC Genome Browser
on Human Feb. 2009 (GRCh37/hg19) Assembly), Length 59 bp
SEQ ID NO: 73
  721 AGCATGGCCCACGCGCACGGGCTGCCGTCGCACATGGGCTGCATGAGCGACGTGGACGCC
          ||:||||:::|++++:|++||:||:++|++:|:|||||:||:|||||++|++||||++:+
  721 AGTATGGTTTACGCGTACGGGTTGTCGTCGTATATGGGTTGTATGAGCGACGTGGACGTC
SEQ ID NO: 74
  781 GACCCGCGGGACCTGGAGGCATTCGCCGAGCGCTTCAAGCAGCGACGCATCAAGCTGGGG
          +|::++++|||::||||||:|||++|++||++:||:|||:||++|++:||:|||:|||||
  781 GATTCGCGGGATTTGGAGGTATTCGTCGAGCGTTTTAAGTAGCGACGTATTAAGTTGGGG
(SEQ ID NO: 75)
F: GGTTGTCGTCGTATATGGGTTGT
(SEQ ID NO: 76)
R: CCCAACTTAATACGTCGCTACTTAAAA
Conditions:
POU4F f2-r2, MSHRM MM, Mg3mM, 58 deg., 5, 5, 10 sec. pr. cycle
Fragment SLC38A4: chr12: 47, 224, 928-47, 225, 029 (UCSC Genome Browser
on Human Feb. 2009 (GRCh37/hg19) Assembly), Length 102 bp
SEQ ID NO: 77
  301 ATTCGCCGTTTTCCCCCACAACCGGCACCTGCCCTGGCCCAGAGCGCAGCGTCCACCTGT
          |||++:++||||:::::|:||:++|:|::||:::|||:::||||++:||++|::|::|||
  301 ATTCGTCGTTTTTTTTTATAATCGGTATTTGTTTTGGTTTAGAGCGTAGCGTTTATTTGT
SEQ ID NO: 78
  361 ACCACCGCTAGATGAAGAGTACCTCACCGCGCGCTGCCTGGGCGCACAGCTGGTGTGCCG
          |::|:++:|||||||||||||::|:|:++++++:||::||||++:|:||:|||||||:++
  361 ATTATCGTTAGATGAAGAGTATTTTATCGCGCGTTGTTTGGGCGTATAGTTGGTGTGTCG
  421 ACCACCGCTAGATGAAGAGTACCTCACCGCGCGCTGCCTGGGCGCACAGCTGGTGTGCCG
          ++:|++::|::|:|::||||:|:|||||||:|::++|:|||++:||:|++|||:||::++
  421 CGTTCGTTATTTTTTTTGGATTTAAGGGTGTTTTCGGTTTACGTTTTTCGGGGTTTTTCG
(SEQ ID NO: 79)
F: TAATCGGTATTTGTTTTGGTTTAGAG
(SEQ ID NO: 80)
R: AACGCGACACACCAACTATAC
Conditions:
SLC38A4 f3-r3, MSHRM MM, Mg3mM, 64 deg., 10, 10, 15 sec. pr. cycle
Fragment TMEM132D: chr12: 130, 387, 867-130, 387, 972 (UCSC Genome
Browser on Human Feb. 2009 (GRCh37/hg19) Assembly), Length 106 bp
SEQ ID NO: 81
  241 TGGTGCCACAGCGTCCCCATCTCAGACGGGCACATCCTGGAGACCCGGAGCGCAGATCCT
          |||||::|:||++|::::||:|:|||++||:|:||::||||||::++|||++:||||::|
  241 TGGTGTTATAGCGTTTTTATTTTAGACGGGTATATTTTGGAGATTCGGAGCGTAGATTTT
SEQ ID NO: 82
  301 CCGCTCCCCGGCGCCGTCCAGGCGAACAAGAGACCGTCTCAGTCCCCTAGAGGCCCGCAG
          :++:|:::++|++:++|::|||++||:||||||:++|:|:|||::::||||||::++:||
  301 TCGTTTTTCGGCGTCGTTTAGGCGAATAAGAGATCGTTTTAGTTTTTTAGAGGTTCGTAG
  361 CGGGGCCGGTGGCGAGGGAGCGCCCGGCTAGGGGCCCGAGCAGCCCGGGCGCCCTGCTCC
          ++|||:++||||++||||||++::++|:||||||::++||:||::++||++:::||:|::
  361 CGGGGTCGGTGGCGAGGGAGCGTTCGGTTAGGGGTTCGAGTAGTTCGGGCGTTTTGTTTT
(SEQ ID NO: 83)
F: TATTTTAGACGGGTATATTTTGGAGATT
(SEQ ID NO: 84)
R: CCGCTACGAACCTCTAAAAAACTAAA
Conditions:
TMEM132D fl-r1, MSHRM MM, Mg3mM, 60 deg., 10,10,15 sec. pr. Cycle
```

### Items

The following items represent specific preferred embodiments of the present invention.

1. A method of determining breast cancer, a predisposition to breast cancer, the prognosis of a breast cancer, and/or monitoring a breast cancer in a subject, said method comprising in a sample from said subject determining the methylation status of at least one gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).
2. The method according to any of the preceding items, wherein the methylation status of at least two gene loci are determined, such as at least three genes, such as at least four gene loci.
3. The method according to any of the preceding items, wherein said methylation status is determined in gene loci FLJ3247, HOXB13 and/or NR2E1.
4. The method according to any of the preceding items, wherein said sample comprise breast tissue, such as breast cells and/or genetic material of breast cells.
5. The method according to any of the preceding items, wherein said sample is a formalin-fixed paraffin-embedded (ffpe) sample.
6. The method according to any of the preceding items, wherein said sample is a bodily fluid, such as a blood sample or a plasma sample.
7. The method according to any of the preceding items, wherein said methylation status is determined by any method selected from the group consisting of Methylation-Specific PCR (MSP), Whole genome bisulfite sequencing (BS-Seq), HELP assays, ChIP-on-chip assays, Restriction landmark genomic scanning, Methylated DNA immunoprecipitation (MeDIP), Pyrosequencing of bisulfite treated DNA, Molecular break light assays, and Methyl Sensitive Southern Blotting.
8. The method according to any of the preceding items, wherein methylation status is determined by methylation specific PCR, bisulfite sequencing, COBRA, melting curve analysis, or DNA methylation arrays.
9. The method according to any of the preceding items, wherein said methylation status is determined by melting curve analysis, such as high resolution melting (HMR) analysis.
10. The method according to any of the preceding items, wherein said methylation status is determined by a method comprising the steps of
i) providing a sample, such as a breast tissue sample, from said subject comprising nucleic acid material comprising said gene locus,
ii) modifying said nucleic acid using an agent which modifies unmethylated cytosine or cleaves nucleic acid sequences in a methylation-dependent manner,
iii) amplifying at least one portion of said gene locus using primers, which span or comprise at least one CpG dinucleotide in said gene locus in order to obtain an amplification product, and
iv) analyzing said amplification product for the presence of modified and/or unmodified cytosine residues, wherein the presence of modified cytosine residues are indicative of methylated cytosine residues.
11. The method according to any of the preceding items, wherein said amplified CpG-containing nucleic acid is analyzed by melting curve analysis
12. The method according to any of the preceding items, wherein said unmethylated cytosine is modified by bisulfite.
13. The method according to any of the preceding items, wherein said methylation status is determined by amplifying at least one portion of said gene locus using at least one primer pair selected from the nucleic acid sequences set forth in table 2 (SEQ ID NO: SEQ ID NO: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83 and 84).
14. The method according to any of the preceding items, wherein said methylation status is determined by amplifying at least one portion of said at least one gene locus, and wherein the amplified portion is detected using at least one oligonucleotide probe.
15. The method according to any of the preceding items, wherein said oligonucleotide probe hybridizes to a sequence selected from the group consisting of SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand) (Table 2).
16. The method according to any of the preceding items, wherein said oligonucleotide probe comprises 10-100 consecutive nucleic acids selected from the group of sequences consisting SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand) (Table 2).
17. The method according to any of the preceding items, wherein a level of methylation positive alleles for said gene locus above the level indicated in table 1, column 4, such as above the level indicated in table 1, column 6, is indicative of breast cancer or a predisposition for breast cancer (for example for HMX2, a level of methylation positive alleles above 0%, such as above 65.2% is indicative of breast cancer or a predisposition for breast cancer).
18. A method for categorizing or predicting the clinical outcome of a breast cancer of a subject, said method comprising in a sample from said subject determining the methylation status of at least one gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).
19. The method according to item 18, wherein the presence of methylation is indicative of decreased overall survival, different stage cancer.
20. A method of evaluating the risk for a subject of contracting cancer, said method comprising in a sample from said subject determining the methylation status of a gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).
21. A method of treating a breast cancer in a human subject, said method comprising the steps of
i. determining breast cancer, a predisposition to breast cancer, or the prognosis of a breast cancer in a subject by a method as defined in any of the preceding items,
ii. selecting human subjects having breast cancer, a predisposition to breast cancer, or a negative or positive prognosis of a breast cancer,
iii. subjecting said subjects identified in step ii. to a suitable treatment for breast cancer.
22. The method according to item 21, wherein said treatment is surgery, chemotherapy and/or radiotherapy.
23. A kit for determining breast cancer, predisposition to breast cancer, or categorizing or predicting the clinical outcome of a breast cancer, or monitoring the treatment of a breast cancer, said kit comprising
i. an agent that (a) modifies methylated cytosine residues but not non-methylated cytosine residues; or (b) modifies non-methylated cytosine residues but not methylated cytosine residues; or (c) modifies a nucleic acid sequence in a methylation-dependent manner,
ii. and at least one pair of oligonucleotide primers that specifically hybridizes under amplification conditions to a region of a gene locus selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81).
24. The kit according to item 23, wherein said at least one primer pair selected from Table 2 (i.e at least one primer pair identified as SEQ ID NO: SEQ ID NO: 3/4, 7/8, 11/12, 15/16, 19/20, 23/24, 27/28, 31/32, 35/36, 39/40, 43/44, 47/48, 51/52, 55/56, 59/60, 63/64, 67/68, 71/72, 75/76, 79/80 and/or 83/84).
25. The kit according to any of items 21 to 24, wherein said kit comprise at least one oligonucleotide probe comprising 10-100 consecutive nucleic acids selected from the group of sequences consisting SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand) (Table 2).
26. The kit according to any of items 21 to 25, wherein said kit comprise at least one oligonucleotide probe which hybridizes to a sequence selected from the group consisting of SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand) (Table 2).
27. The kit according to any of items 21 to 26, said kit further comprising a DNA polymerase.
28. The kit according to any of items 21 to 27, wherein said agent is a bisulfite, hydrogen sulfite, and/or disunite reagent, for example sodium bisulfite.
29. The kit according to any of items 21 to 27, said kit further comprising a methylation-sensitive restriction enzyme.
30. Use of oligonucleotide primers comprising a sequence, which is a subsequence of a gene loci selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81) or the complement thereof for diagnosing breast cancer in a method of any of the preceding items.
31. The use according to any of the preceding items, wherein said oligonucleotide primers is selected from the nucleic acid sequences set forth in table 2 (SEQ ID NO: SEQ ID NO: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83 and 84).
32. The use according to any of the preceding items, wherein said gene locus is selected from the group consisting of FLJ3247, HOXB13 and NR2E1.
33. A method of identifying therapeutically effective agents for treatment of breast cancer, said method comprising
i. providing a breast cancer cell line comprising one or more genetic loci selected from the group consisting of BC008699 (SEQ ID NO: 1), CA10 (SEQ ID NO: 5), FLJ32447 (SEQ ID NO: 9), HMX2 (SEQ ID NO: 13), HS3ST2 (SEQ ID NO: 17), LHX1 (SEQ ID NO: 21), NR2E1 (SEQ ID NO: 25), PHOX2B (SEQ ID NO: 29), SIX6 (SEQ ID NO: 33), TITF1 (SEQ ID NO: 37), WT1 (SEQ ID NO: 41), BX161496 (SEQ ID NO: 45), CHR (SEQ ID NO: 49), GHSR (SEQ ID NO: 53), HOXB13 (SEQ ID NO: 57), HTR1B (SEQ ID NO: 61), NKX2-3 (SEQ ID NO: 65), ONECUT (SEQ ID NO: 69), POU4F (SEQ ID NO: 73), SLC38A4 (SEQ ID NO: 77) and TMEM132D (SEQ ID NO: 81),
ii. providing one or more potential therapeutic agents,
iii. treating said breast cancer cells by bringing said agents in contact with said breast cancer cells,
iv. determining methylation status of said one or more genetic loci
v. comparing said methylation status of said treated breast cancer cells with the methylation status of said breast cancer cells, when untreated, wherein a decreased level of methylation positive alleles is indicative of a therapeutic agent.

1. A method of determining breast cancer, a predisposition to breast cancer, the prognosis of a breast cancer, and/or monitoring a breast cancer in a subject, said method comprising in a sample from said subject determining the methylation status of at least one gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61).

2. A method for assessing whether a human subject is likely to develop breast cancer, said method comprising
i) providing a sample from said human subject,
ii) determining in said sample the methylation status of at least one gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61),
iii) on the basis of said methylation status identifying a human subject that is more likely to develop breast cancer.

3. The method according to any of the preceding claims, wherein the methylation status of at least two gene loci are determined, such as at least three genes, such as at least four gene loci.

4. The method according to any of the preceding claims, wherein said methylation status is determined in the PHOX2B gene locus and at least one additional gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5) and GHSR (SEQ ID NO: 53).

5. The method according to any of the preceding claims, wherein said methylation status is determined in one or more gene loci selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9) and/or CHR (SEQ ID NO: 49).

6. The method according to any of the preceding claims, wherein said methylation status is determined in one or more gene loci selected from the group consisting of TMEM132D (SEQ ID NO: 81), TITF1 (SEQ ID NO: 37), NR2E1 (SEQ ID NO: 25), CA10 (SEQ ID NO: 5) and/or GHSR (SEQ ID NO: 53).

7. The method according to any of the preceding claims, wherein said sample comprise breast tissue, such as breast cells and/or genetic material of breast cells.

8. The method according to any of the preceding claims, wherein said sample is a formalin-fixed paraffin-embedded (ffpe) sample.

9. The method according to any of the preceding claims, wherein said sample is a bodily fluid, such as a blood sample or a plasma sample.

10. The method according to any of the preceding claims, wherein said methylation status is determined by any method selected from the group consisting of Methylation-Specific PCR (MSP), Whole genome bisulfite sequencing (BS-Seq), HELP assays, ChIP-on-chip assays, Restriction landmark genomic scanning, Methylated DNA immunoprecipitation (MeDIP), Pyrosequencing of bisulfite treated DNA, Molecular break light assays, and Methyl Sensitive Southern Blotting.

11. The method according to any of the preceding claims, wherein methylation status is determined by methylation specific PCR, bisulfite sequencing, COBRA, melting curve analysis, or DNA methylation arrays.

12. The method according to any of the preceding claims, wherein said methylation status is determined by melting curve analysis, such as high resolution melting (HMR) analysis.

13. The method according to any of the preceding claims, wherein said methylation status is determined by a method comprising the steps of
i) providing a sample, such as a breast tissue sample, from said subject comprising nucleic acid material comprising said gene locus,
ii) modifying said nucleic acid using an agent which cleaves nucleic acid sequences in a methylation-dependent manner,
iii) amplifying at least one portion of said gene locus using primers, which span or comprise at least one CpG dinucleotide in said gene locus in order to obtain an amplification product, and
iv) analyzing said amplification product.

14. The method according to claim 13, wherein said amplification product is analysed by detecting the presence or absence of amplification product, wherein the presence of amplification product indicates that the target nucleic acid has not been cleaved by said agent, and wherein the absence of amplification product indicates that the target nucleic acid has been cleaved by said agent.

15. The method according to any of the preceding claims, wherein said methylation status is determined by a method comprising the steps of
i) providing a sample, such as a breast tissue sample, from said subject comprising nucleic acid material comprising said gene locus,
ii) modifying said nucleic acid using an agent which modifies unmethylated cytosine,
iii) amplifying at least one portion of said gene locus using primers, which span or comprise at least one CpG dinucleotide in said gene locus in order to obtain an amplification product, and
iv) analyzing said amplification product.

16. The method according to claim 15, wherein said amplification product is analysed for nucleic acid substitutions resulting from conversion of modified cytosine residues, wherein the presence of converted cytosine residues are indicative of unmethylated cytosine residues, and presence of unconverted cytosine residues is indicative of methylated cytosine residues.

17. The method according to any of the preceding claims, wherein said amplified CpG-containing nucleic acid is analyzed by melting curve analysis

18. The method according to any of the preceding claims, wherein said unmethylated cytosine is modified by bisulfite.

19. The method according to any of the preceding claims, wherein said methylation status is determined by amplifying at least one portion of said gene locus using at least one primer pair selected from the nucleic acid sequences set forth in table 2 (SEQ ID NO: SEQ ID NO: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83 and 84).

20. The method according to any of the preceding claims, wherein said methylation status is determined by amplifying at least one portion of said at least one gene locus, and wherein the amplified portion is detected using at least one oligonucleotide probe.

21. The method according to any of the preceding claims, wherein said oligonucleotide probe hybridizes to a sequence selected from the group consisting of SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand) (Table 2).

22. The method according to any of the preceding claims, wherein said oligonucleotide probe comprises 10-100 consecutive nucleic acids selected from the group of sequences consisting SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand) (Table 2).

23. The method according to any of the preceding claims, wherein a level of methylation positive alleles for said gene locus above the level indicated in table 1, column 4, such as above the level indicated in table 1, column 6, is indicative of breast cancer or a predisposition for breast cancer (for example for HMX2, a level of methylation positive alleles above 0%, such as above 65.2% is indicative of breast cancer or a predisposition for breast cancer).

24. A method for categorizing or predicting the clinical outcome of a breast cancer of a subject, said method comprising in a sample from said subject determining the methylation status of at least one gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61).

25. The method according to claim 24, wherein the presence of methylation is indicative of decreased overall survival, different stage cancer.

26. A method of evaluating the risk for a subject of contracting cancer, said method comprising in a sample from said subject determining the methylation status of a gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61).

27. A method of treating a breast cancer in a human subject, said method comprising the steps of
i. determining breast cancer, a predisposition to breast cancer, or the prognosis of a breast cancer in a subject by a method as defined in any of the preceding claims,
ii. selecting human subjects having breast cancer, a predisposition to breast cancer, or a negative or positive prognosis of a breast cancer,
iii. subjecting said subjects identified in step ii. to a suitable treatment for breast cancer.

28. The method according to claim 27, wherein said treatment is surgery, chemotherapy and/or radiotherapy.

29. A kit for determining breast cancer, predisposition to breast cancer, or categorizing or predicting the clinical outcome of a breast cancer, or monitoring the treatment of a breast cancer, said kit comprising
i. an agent that (a) modifies methylated cytosine residues but not non-methylated cytosine residues; or (b) modifies non-methylated cytosine residues but not methylated cytosine residues; or (c) modifies a nucleic acid sequence in a methylation-dependent manner,
ii. and at least one pair of oligonucleotide primers that specifically hybridizes under amplification conditions to a region of a gene locus selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61).

30. The kit according to claim 29, wherein said at least one primer pair selected from Table 2, for example at least one primer pair identified as SEQ ID NO: SEQ ID NO: 3/4, 7/8, 11/12, 15/16, 19/20, 23/24, 27/28, 31/32, 35/36, 39/40, 43/44, 47/48, 51/52, 55/56, 59/60, 63/64, 67/68, 71/72, 75/76, 79/80 and/or 83/84.

31. The kit according to any of claims 27 to 30, wherein said kit comprise at least one oligonucleotide probe comprising 10-100 consecutive nucleic acids selected from the group of sequences consisting SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand) (Table 2).

32. The kit according to any of claims 27 to 31, wherein said kit comprise at least one oligonucleotide probe which hybridizes to a sequence selected from the group consisting of SEQ ID NO: 1, 5, 9, 13, 17, 21, 25, 29, 33, 37, 41, 45, 49, 53, 57, 61, 65, 69, 73, 77 and/or 81 and/or the complement thereof (non-modified strand) or the group consisting of SEQ ID NO: 2, 6, 10, 14, 18, 22, 26, 30, 34, 38, 42, 46, 50, 54, 58, 62, 66, 70, 74, 78 and/or 82 and/or the complement thereof (modified strand) (Table 2).

33. The kit according to any of claims 27 to 32, said kit further comprising a DNA polymerase.

34. The kit according to any of claims 27 to 33, wherein said agent is a bisulfite, hydrogen sulfite, and/or disulfite reagent, for example sodium bisulfite.

35. The kit according to any of claims 27 to 33, said kit further comprising a methylation-sensitive restriction enzyme.

36. Use of oligonucleotide primers comprising a sequence, which is a subsequence of a gene loci selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61) or the complement thereof for diagnosing breast cancer in a method of any of the preceding claims.

37. The use according to any of the preceding claims, wherein said oligonucleotide primers is selected from the nucleic acid sequences set forth in table 2 (SEQ ID NO: SEQ ID NO: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83 and 84).

38. The use according to any of the preceding claims, wherein said gene locus is selected from the group consisting of FLJ3247, HOXB13 and NR2E1.

39. A method of identifying therapeutically effective agents for treatment of breast cancer, said method comprising
i. providing a breast cancer cell line comprising one or more genetic loci selected from the group consisting of PHOX2B (SEQ ID NO: 29), POU4F (SEQ ID NO: 73), SIX6 (SEQ ID NO: 33), WT1 (SEQ ID NO: 41), ONECUT (SEQ ID NO: 69), NKX2-3 (SEQ ID NO: 65), FLJ32447 (SEQ ID NO: 9), CHR (SEQ ID NO: 49), TMEM132D (SEQ ID NO: 81), TITF1 (SEQ ID NO: 37), NR2E1 (SEQ ID NO: 25), CA10 (SEQ ID NO: 5), GHSR (SEQ ID NO: 53), BC008699 (SEQ ID NO: 1), HS3ST2 (SEQ ID NO: 17), LHX1 (SEQ ID NO: 21), BX161496 (SEQ ID NO: 45), SLC38A4 (SEQ ID NO: 77), HMX2 (SEQ ID NO: 13), HOXB13 (SEQ ID NO: 57) and HTR1B (SEQ ID NO: 61),
ii. providing one or more potential therapeutic agents,
iii. treating said breast cancer cells by bringing said agents in contact with said breast cancer cells,
iv. determining methylation status of said one or more genetic loci
v. comparing said methylation status of said treated breast cancer cells with the methylation status of said breast cancer cells, when untreated, wherein a decreased level of methylation positive alleles is indicative of a therapeutic agent.
